(19) United States
(12) Patent Application Publication
(10) Pub. No.: US 2023/0204565 A1
(43) Pub. Date: Jun. 29, 2023

(54) Methods for Culturing Cancer Cells and for Inhibiting Invasion of Cancer

(71) Applicant: The Regents of the University of California, Oakland, CA (US)

(72) Inventors: Aparna Bhaduri, San Francisco, CA (US); Elizabeth Di Lullo, San Francisco, CA (US); Arnold R. Kriegstein, Mill Valley, CA (US)

(21) Appl. No.: 17/785,818

(22) PCT Filed: Dec. 23, 2020
(86) PCT No.: PCT/US2020/066924

§ 371 Date: Jun. 15, 2022

Related U.S. Application Data
(60) Provisional application No. 62/978,713, filed on Feb. 19, 2020.
(60) Provisional application No. 62/956,508, filed on Jan. 2, 2020.

(51) Int. Cl. G01N 33/50; C12N 5/09; C12N 5/079

(52) U.S. Cl. CPC G01N 33/5011; C12N 5/0618; C12N 5/0693; C12N 2501/15; C12N 2501/415; C12N 2506/02; C12N 2506/45; C12N 2513/00

(57) ABSTRACT

The present disclosure provides a brain organoid for culturing cancer cells for a prolonged period of time and methods for culturing cancer cells in a brain organoid for a period of time at least one week. The cancer cells may be primary cancer cells obtained from a cancer from a subject. The brain organoid may be generated from embryonic stem cells or induced pluripotent stem cells that are not transformed to render them oncogenic. In certain aspects, the cancer cells cultured in the brain organoid may be Protein Tyrosine Phosphatase Receptor Type Z1 (PTPRZ1) expressing cancer cells obtained from a cancer from a subject. Also provided are methods for inhibiting tumor invasion in a cancer of a nervous system by administering to a subject suffering from such cancer an inhibitor of the PTPRZ1 pathway and for screening for inhibitors of cancer cell growth and/or invasion.

### CROSS-REFERENCE TO RELATED APPLICATIONS

This application claims the benefit of U.S. Provisional Pat. Application No. 62/956,508, filed Jan. 2, 2020, and U.S. Provisional Pat. Application No. 62/978,713, filed Feb. 19, 2020, which applications are incorporated herein by reference in their entireties.

### STATEMENT OF GOVERNMENT SUPPORT

This invention was made with government support under grants R35 NS097305 and R01 NS091544 awarded by The National Institutes of Health. The government has certain rights in the invention.

### INTRODUCTION

Glioblastoma is an aggressive form of cancer and the most common primary malignant brain cancer in adults. The ability of glioblastoma cells to migrate through brain tissue to distant regions outside of the tumor bulk is thought to confer resistance to standard-of-care treatment by impeding complete surgical resection. Glioblastomas inevitably recur after surgery.

Similarities between cancer and development have long been appreciated. In the 19^{th} century, pathologists noted morphological similarities between developing tissue and tumor tissue, leading to the ‘embryonic rest’ hypothesis proposing that remnants of developmental tissue stay dormant in the adult and become reactivated in cancer. More recently, the concept that brain tumors are maintained by a stem cell-like population has become well established. Populations of cells harboring a stemness signature have been identified in glioblastoma, and recent studies in astrocytomas and oligodendrogliomas suggest that lineage trajectories observed during normal astrocyte and oligodendrocyte development may be replicated by cells in these cancers. However, identifying the cell types that comprise the tumor bulk and the developmental trajectories underlying the generation of heterogeneous tumors like glioblastoma remains elusive. Understanding the molecular drivers of intra-glioblastoma heterogeneity is essential to developing novel therapeutic strategies for patients, but technical limitations including suboptimal preclinical models, have delayed scientific and clinical progress.

Single-cell RNA sequencing (scRNA-seq) offers an opportunity to explore the cell type composition of primary glioblastoma resection specimens, thereby overcoming the technical barriers that have hampered understanding of intra-glioblastoma heterogeneity. Prior studies exploring glioblastoma at the single-cell level have primarily focused on immune and infiltrating cell populations or have specifically targeted a subset of readily identifiable cell types. A recent study of adult and pediatric glioblastoma used single-cell sequencing to characterize four primary cell states of a tumor but did not characterize tumor cell types as they compare to primary developing or adult human brain, leaving open the question of what cell types actually comprise the tumor bulk. Identifying the cell types that compose the tumor and how they arise from GSCs is desired to identify activated gene signatures that could provide therapeutic targets to inhibit tumor growth.

### SUMMARY

The present disclosure provides a brain organoid for culturing cancer cells for a prolonged period of time and methods for culturing cancer cells in a brain organoid for a prolonged period of time. The cancer cells may be cultured in the brain organoid as disclosed herein for at least one week, e.g., e.g., at least two weeks, at least three weeks, at least four weeks, or at least 60 days, or more. The cancer cells may be primary cancer cells obtained from a cancer from a subject. The brain organoid may be generated from embryonic stem cells or induced pluripotent stem cells that are not transformed to render them oncogenic. In certain aspects, the cancer cells cultured in the brain organoid may be Protein Tyrosine Phosphatase Receptor Type Z1 (PTPRZ1) expressing cancer cells obtained from a cancer from a subject. In certain aspects, the cancer may be a cancer of the nervous system. In certain aspects, the brain organoid may be a cortical organoid. In certain embodiments, a method for screening agents that can inhibit the growth of cancer cells cultured in a brain organoid, such as a cortical organoid, is provided.

This disclosure identifies PTPRZ1 expressing outer radial glia (oRG) like cells as a subtype of glioblastoma stem cells that drive tumor progression and invasion in a cancer of the nervous system, such as, brain cancer, particularly, glioblastoma. Aspects of the present disclosure provide a method for screening agents that can inhibit PTPRZ1 pathway in PTPRZ1 expressing cells. The brain organoids and culture methods using the same as provided herein may be utilized for such screens.

The disclosure also provides that inhibiting the PTPRZ1 pathway can be used to inhibit tumor progression and invasion of a cancer of the nervous system, such as, brain cancer, particularly, glioblastoma. Accordingly, certain embodiments of the invention provide a method for inhibiting invasion of a cancer of the nervous system by administering to a subject suffering from such cancer an inhibitor of the PTPRZ1 pathway. An inhibitor of the PTPRZ1 pathway can be an inhibitor of the activity or expression of PTPRZ1 or an inhibitor of the activity or expression of the ligand of PTPRZ1 , pleiotrophin (PTN). In preferred embodiments, an inhibitor of PTPRZ1 pathway is administered locally in and around the cancerous tissue of the subject or delivered into the cancer via vehicles specifically targeted to the cancer cells.

An inhibitor of PTPRZ1 pathway can be an antibody or an antigen binding fragment of an antibody against PTPRZ1 , an antibody or an antigen binding fragment of an antibody against PTN, an oligonucleotide that specifically inhibits the expression of PTPRZ1 protein or its mRNA, an oligonucleotide that specifically inhibits the expression of PTN protein or its mRNA, a small molecule that specifically inhibits the activity of PTPRZ1, or a small molecule that specifically inhibits the activity of PTN.

### BRIEF DESCRIPTION OF THE DRAWINGS

Copies of this patent or patent application publication with color drawing(s) will be provided by the Office upon request and payment of the necessary fee.

FIG. 1A. Single cell RNA-sequencing of primary glioblastoma tumors creates an atlas of tumor cell types. Schematic of the workflow used to create the tumor atlas. Primary tumor resections are obtained and dissociated for single-cell sequencing. Clustering is performed and cell clusters are compared to annotated clusters from adult and developing human datasets.

FIG. 1B. Single cell RNA-sequencing of primary glioblastoma tumors creates an atlas of tumor cell types. tSNE plots showing the clustering of primary tumor cells, colored by cluster, tumor of origin, and annotated cell type.

FIG. 1C. Single cell RNA-sequencing of primary glioblastoma tumors creates an atlas of tumor cell types. Similarity matrix of clusters from primary glioblastoma analysis, correlated in the space of marker genes. Heatmap color indicates the Pearson’s correlation, and above the heatmap is the annotated cell type for each cluster. To the right of the heatmap are marker gene violin plots depicting the distribution of cell type genes across clusters. Some genes (such as GFAP) are broadly expressed but strongly enriched in clusters where the marker is associated with cell type (such as cluster 15, protoplasmic astrocyte-like cells).

FIG. 1D. Single cell RNA-sequencing of primary glioblastoma tumors creates an atlas of tumor cell types. Composition of individual tumors is shown as a proportion of single cells identified as each annotated cell type. Stacked barchart shows the proportional composition for each tumor, and graph to the right shows the number of cell types represented in each tumor.

FIG. 2A. Glioblastoma Cancer Stem Markers are Expressed in a Variety of Cell Types. Feature plots of selected cancer stem markers. Some stemness genes (such as SOX2 and CD44) are widely expressed, while others (such as PROM1 and FUT4) mark rare populations.

FIG. 2B. Glioblastoma Cancer Stem Markers are Expressed in a Variety of Cell Types. Heatmap of normalized gene counts for glioblastoma cancer stem cell (GSC) markers with blue corresponding to no expression and red corresponding to 2.5 or more normalized counts within the cell. Many markers are co-expressed, and GSC markers span a variety of cell types (annotated above the heatmap).

FIG. 2C. Glioblastoma Cancer Stem Markers are Expressed in a Variety of Cell Types. Fraction of cells annotated by cell type (based upon clustering analysis) that comprise GSC marker positive cells (based upon individual cell annotations), organized first by marker. Using markers that have been characterized in primary glioblastoma tumors and exist in tumor bulk populations, GSCs were identified for each tumor (based upon individual cell annotations). The heterogeneous composition of cell types for GSCs within a single is shown on the right.

FIG. 2D. Glioblastoma Cancer Stem Markers are Expressed in a Variety of Cell Types. The expression of the radial glia gene signature was evaluated to be significantly higher in GBM radial glia-like cells compared to all other cell types (**** = p < 0.0001, Student’s two-sided t-test). GBM radial glia-like cells were compared to the known molecular signatures of radial glia subtypes. The arrows pointing to outer radial glia (oRG), truncated radial glia (tRG) and ventricular radial glia (vRG) are weighted in their thickness proportional to the relative correlation of GBM developmental radial glia-like cells to each subtype. The graph shows a significantly higher correlation (**** = p <0.0001, Student’s two-sided t-test using all 32,000 cell observations) to oRG than to the other subtypes. Network diagram depicts the oRG network (Pollen et al. 2015) that is highly correlated (R > 0.30) in GBM single-cell data. Red highlights nodes with > 20 connections, and orange highlights those with greater than 10 connections. Most genes in the developmental oRG network are preserved in GBM.

FIG. 3A. Copy Number Analysis Demonstrates Enrichment of Progenitor Cell Types in Tumor Cells. Quantification of the proportion of cells by tumor that are designated as normal or tumor cells based upon copy number variation (CNV) analysis. The fraction of all cells is shown by annotated cell type in a stack barchart.

FIG. 3B. Copy Number Analysis Demonstrates Enrichment of Progenitor Cell Types in Tumor Cells. The fraction of radial glia-like cells annotated as normal or tumor cells, mean with standard deviation is shown. (* = p <0.05, Student’s two-sided t-test).

FIG. 3C. Copy Number Analysis Demonstrates Enrichment of Progenitor Cell Types in Tumor Cells. Reconstructions of the relationship between cells within the tumor are shown in phylogenies. Phylogenies were reconstructed based upon parsimony based upon the CNV calls. On each branch of the phylogeny, proportions of the annotated cell types for each CNV event are shown in horizontal stacked barcharts.

FIG. 4A. GBM oRG-like cells undergo mitotic somal translocation and can give rise to proliferative daughter cells. Schematic of the workflow used to set up live imaging analysis of primary tumor resections. Tumors were dissociated and plated on Matrigel, after which they were infected with an adenovirus to express GFP. Live imaging was performed over the course of 72 hours.

FIG. 4B. GBM oRG-like cells undergo mitotic somal translocation and can give rise to proliferative daughter cells. Still images of videos of time-lapse imaging depict a glioblastoma cell undergoing a mitotic somal translocation (MST) as seen by the translocation of the soma followed by cytokinesis.

FIG. 4C. GBM oRG-like cells undergo mitotic somal translocation and can give rise to proliferative daughter cells. Box plot (min to max) shows the somal translocation distance in the observed MSTs. These distances and distribution are comparable to normal development. Distances calculated from 5 biological replicates and 3-8 imaging positions each.

FIG. 4D. GBM oRG-like cells undergo mitotic somal translocation and can give rise to proliferative daughter cells. Another two examples of MST with the cleavage plane annotated to depict the different angles observed.

FIG. 4E. GBM oRG-like cells undergo mitotic somal translocation and can give rise to proliferative daughter cells. Pie chart shows the proportion of horizontal, vertical, and oblique cleavage angles observed (relative to primary fiber) in live imaging analysis. 43 observations were analyzed across 5 biological replicates.

FIG. 4F. GBM oRG-like cells undergo mitotic somal translocation and can give rise to proliferative daughter cells. Still images from live imaging analysis that identifies MST divisions giving rise to proliferative daughter cells in the same frame.

FIG. 5A. Sorting for PTPRZ1 positive cells enriches for oRG-like cells. Still images of mitotic somal translocations in cells that were sorted for PTPRZ1 expression from a primary tumor.

FIG. 5B. Sorting for PTPRZ1 positive cells enriches for oRG-like cells. Schematic shows the process of transplantation. Primary tumor cells are sorted for PTPRZ1 positive populations or unsorted populations and are sampled with single-cell RNA sequencing and remaining cells are infected with adenovirus with GFP labeling. These cells are placed upon cortical organoids aged between week 6 and 10. After two weeks in the organoid, GFP positive cells are FACS sorted and analyzed with single-cell RNA sequencing.

FIG. 5C. Sorting for PTPRZ1 positive cells enriches for oRG-like cells. Immunofluorescence of organoid (SF12011 transplanted) show GFP positive cells, and a subset of these cells co-express PTPRZ1.

FIG. 5D. Sorting for PTPRZ1 positive cells enriches for oRG-like cells. Single-cell RNA sequencing was performed prior to transplant from 3 primary tumors and 2 weeks after cells were transplanted and FACS sorted from the organoid. Positive sort significantly enriches for radial glia-like cells (* p-value < 0.05, Welch’s t-test). Stacked barchart depicts proportion of cells that correspond to broad cell types. Both PTPRZ1 + and PTPRZ1 - cells give rise to a variety of cell types that do not exist in the original population including differentiated populations of neurons and astrocytes.

FIG. 5E. Sorting for PTPRZ1 positive cells enriches for oRG-like cells. For each subpopulation, the subset of cells that express GSC markers is shown. Each of the pre-transplanted populations express high levels of some of these markers, and the expression decreases after transplant, corresponding to the increase in differentiated cell populations in the post-transplanted tumors.

FIG. 5F. Sorting for PTPRZ1 positive cells enriches for oRG-like cells. Workflow of single-cell sequencing is shown. PTPRZ1 KD and a scrambled control were used to knockdown PTPRZ1 in both primary developing human cortex cells and primary glioblastoma cells. The resulting gene expression identified a significant overlap between the PTPRZ1 downregulated genes. (* = p-value < 0.05). In both systems, the proportion of radial glia and other progenitor populations (OPC, IPC, dividing progenitors) was also decreased. These changes were accompanied by a significant downregulation of several outer radial glia marker genes.

FIG. 6A. PTPRZ1 Promotes MST driven invasiveness of glioblastoma. Short hairpin induced knockdown of PTPRZ1 (**** = p < 0.0001, Student’s two-sided t-test) decrease the length of somal translocation length using 3 biological replicates and 4 technical replicates each in primary samples and in 4 technical replicates of the DBTRGFL patient derived xenograft line.

FIG. 6B. PTPRZ1 Promotes MST driven invasiveness of glioblastoma. Invasion assays of patient derived xenograft (PDX) line DBTRGFL were performed with control (scrambled shRNA), PTPRZ1 knockdown (shRNA) and Rock Inhibition. Invasions were imaged every 24 hours, and Day 0 and Day 3 are shown. Quantification was performed by calculating how much of the gap was filled. Statistics were performed for both PTPRZ1 knockdown and Rock inhibition, and statistics are showing for Rock inhibition on top and PTPRZ1 knockdown below (* = p <0.05, ** p < 0.01, *** p < 0.001, Student’s two-sided t-test).

FIG. 6C. PTPRZ1 Promotes MST driven invasiveness of glioblastoma. Schematic of the mouse experiments performed in the Examples disclosed herein. PDX line DBTRGFL was historically generated by sampling primary tumor and propagating as a xenograft in the flank of a mouse. This tumor was then sorted for PTPRZ1 positive cells, and positive and unsorted cells of equal numbers were injected into the mouse. The cells were profiled using single-cell sequencing prior to injection, and resultant tumors were profiled after first emergence of tumor (F1) and after serial transplant (F2).

FIG. 6D. PTPRZ1 Promotes MST driven invasiveness of glioblastoma. Single-cell sequencing was performed on samples prior to and after tumor formation. Each cluster was correlated to the closest broad cell type and is shown as a proportion of the whole. PTPRZ1 + cells give rise to cell types not present in the initial sort, including astrocytes and upper layer neurons.

FIG. 6E. PTPRZ1 Promotes MST driven invasiveness of glioblastoma. DBTRGFL is labeled with luciferase and staining of surrounding brain tissue after serial transplantation identifies luciferase positive cells. A subset of these cells are also PTPRZ1 positive. Scale bar = 100 µM.

FIG. 7A. Corresponding single cell RNA-sequencing of primary glioblastoma tumors to known cell types. Correlation matrix of each primary glioblastoma to annotated cell types from adult cortex and development. For each row, the column (col) min to the column max is shown as a gradient from blue to red. The reddest box for each column represents the cell type which most resembles the glioblastoma cluster.

FIG. 7B. Corresponding single cell RNA-sequencing of primary glioblastoma tumors to known cell types. Graph shows the number of tumors in which each cell type has been identified of the 11 tumors sequenced in this study.

FIG. 7C. Corresponding single cell RNA-sequencing of primary glioblastoma tumors to known cell types. Feature plots of marker genes (also shown as violin plots in FIG. 1C) with gray representing no expression and dark purple representing maximal expression.

FIG. 7D. Corresponding single cell RNA-sequencing of primary glioblastoma tumors to known cell types. Stacked barplot of cell type composition for cells analyzed from Neftel et al. 2019, indicating the presence of cell type heterogeneity, including outer radial glia cells.

FIG. 8A. Histopathology of samples validates tumoral heterogeneity and a subset of cell type composition. Tumor hematoxylin and eosin stain of 8 of the tumors sampled in this study. Varied tumor structure and differentiation status is observed, consistent with prior reports of tumor heterogeneity.

FIG. 8B. Histopathology of samples validates tumoral heterogeneity and a subset of cell type composition. Two representative Ki67 stainings are shown that were used to label dividing cells and calculate a mitotic index. Tumors vary in their Ki67 staining (show in bar chart), but all GBM samples consistently have above 20% of cells in mitosis.

FIG. 8C. Histopathology of samples validates tumoral heterogeneity and a subset of cell type composition. Immunofluorescent stains were performed on banked samples corresponding to the tumors that were analyzed. The markers chosen corresponded to markers of major cell populations identified from the MetaCluster analysis. The heatmap on the right summarizes the degree of validation observed in the 8 tumors that were validated, as quantified by the number of positively stained cells within an equal area of tumor. Each tumor expresses at least one of the observed cell types and many have representation from multiple clusters. Scale bar = 100 µM.

FIG. 9A. Expression of glioblastoma cancer stem cell markers in a variety of cell types including IVY database. Additional feature plots of cancer stem cell markers in single-cell RNA sequencing shows both broad and very sparse expression of genes associated with cancer stemness identity.

FIG. 9B. Expression of glioblastoma cancer stem cell markers in a variety of cell types including IVY database. Representative images of immunohistochemistry of a number of markers from the IVY database show both glioblastoma cancer stem cell markers and cell type identification markers can be broadly expressed throughout the tumor, and often co-localize. The cancer stem cell markers are not frequently restricted to a single cell type, as shown by the co-localization of progenitor (HES6) and interneuron (DLX5) expression with the expression of cancer stem cell markers.

FIG. 9C. Expression of glioblastoma cancer stem cell markers in a variety of cell types including IVY database. Heatmap depicting data from IVY Glioblastoma database recapitulates observations in single-cell analysis, with each tumor expressing a heterogeneity of cancer stem cell markers and cell type markers as indicated by the red signal in the heatmap. Genes marked by light blue at the top key across columns indicates normal genes that are used as cell type markers, while the darker blue and green represent Tier 1 and Tier 2 glioblastoma cancer stem cell (GSC) genes respectively. The Tier 1 genes are the most validated GSC genes and Tier 2 are hypothesized to play an important role in GSC biology. The heatmap depicts that within each tumor in each row, expression of a variety of cell identity genes can be co-expressed with many GSC genes.

FIG. 9D. Expression of glioblastoma cancer stem cell markers in a variety of cell types including IVY database. Scatterplots depict the expression of a glioblastoma cancer stem cell marker (x - axis) and the expression of cell type identifier markers (as defined by the normal genes in the above heatmap) (y - axis) as generated from expression data in the IVY Glioblastoma database. Each dot represents a co-expression pattern in a single tumor of a cell type marker with a cancer stem cell marker, with the key for each of the cell type markers shown below. In many cases, glioblastoma cancer stem cell markers co-express within a tumor with a variety of cell type markers.

FIG. 10A. Using single cell RNA-sequencing to identify tumor and normal cells within the dataset. Distribution of copy number by chromosome is observed for each sample. Graph of this distribution as well as the summary barcharts and dot plots used to determine normal vs tumor cell identity is depicted.

FIG. 10B. Using single cell RNA-sequencing to identify tumor and normal cells within the dataset. Binned distribution per chromosome depicts the amplification (red) or deletion (blue) as detected from binned analyses from the transcriptome analysis (top) or from the exome sequencing (bottom). These plots show substantial correspondence between the analyses, validating the efficacy of the approach in the 10X data.

FIG. 10C. Using single cell RNA-sequencing to identify tumor and normal cells within the dataset. Empirical FDR is calculated via permutation analysis and shows an FDR of 0.002.

FIG. 10D. Using single cell RNA-sequencing to identify tumor and normal cells within the dataset. Validation of the CNV analysis from transcriptome as compared to exome analysis shows that > 95% of CNV calls are concordant between analyses, showing the transcriptional analysis is an appropriate way of identifying CNVs and normal cell annotations.

FIG. 10E. Using single cell RNA-sequencing to identify tumor and normal cells within the dataset. Cell type composition of cells annotated as tumor or normal, averaged across all five tumors analyzed with CNV analysis. Normal cells are more enriched for microglia, macrophages and oligodendrocytes while normal cells are enriched for oligodendrocyte precursor cells (OPCs) and radial glia.

FIG. 11A. Enrichment of PTPRZ1 positive cells. Staining of dissociated GBM cells after MST events identifies expression of progenitor markers that are enriched in outer radial glia cells: SOX2, VIM and HOPX.

FIG. 11B. Enrichment of PTPRZ1 positive cells. Violin plot shows expression of PTPRZ1 in GBM developmental radial glia-like cells, primary developmental radial glia and adult temporal lobe samples.

FIG. 11C. Enrichment of PTPRZ1 positive cells. Graph showing the proportion of MST-like division in unsorted and enriched PTPRZ1-positive samples.

FIG. 11D. Enrichment of PTPRZ1 positive cells. Graph depicts the percent of cells that are PTPRZ1-positive and the percent of designated radial glia-like cells that are PTPRZ1-positive. Mean and standard deviation across 11 sequenced tumors shown. On the right, a feature plot depicting PTPRZ1 expression across the 11 tumors is presented.

FIG. 11E. Enrichment of PTPRZ1 positive cells. Fold-change enrichment of outer radial glia genes in the radial glia cell clusters compared to remaining clusters (log₂ value shown).

FIG. 11F. Enrichment of PTPRZ1 positive cells. Representative FACS plots showing the enrichment of PTPRZ1 + cells from resected tumor specimens.

FIG. 11G. Enrichment of PTPRZ1 positive cells. On the left, still images of a movie of light sheet imaging of a cleared organoid 2 weeks after transplant with DBTRGFL unsorted cells that are GFP labeled. On the right, confocal images of cortical organoids being invaded by primary tumor cells from PTPRZ1 +, PTPRZ1- and unsorted tumor samples.

FIG. 11H. Enrichment of PTPRZ1 positive cells. Representative FACS plots are shown for the retrieval of GFP positive cells 2 weeks after organoid transplantation. GFP positive cells were used for single-cell RNA sequencing.

FIG. 111. Enrichment of PTPRZ1 positive cells. Correlation of SF12011 clusters from PTPRZ1-positive post-transplant experiment to each of the original GBM clusters. There is strong correspondence and also heterogeneity that reflects the initial tumoral heterogeneity.

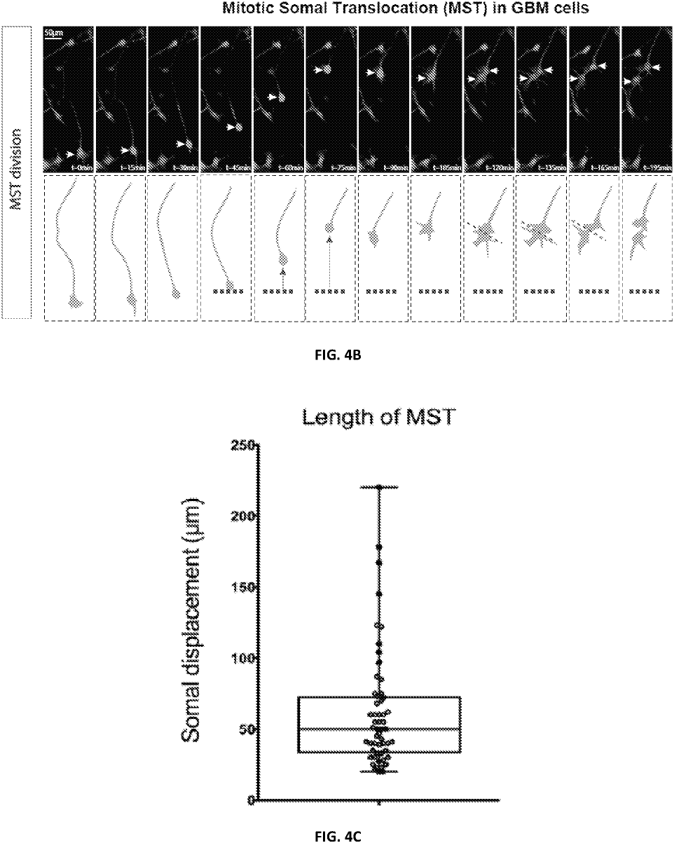

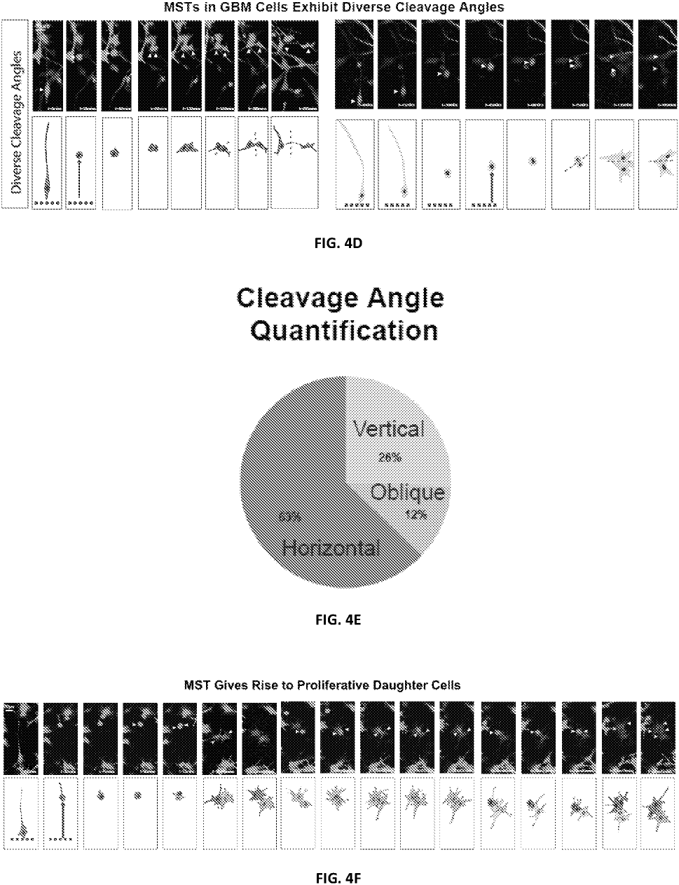

FIG. 12A. Transplants of primary glioblastoma tumors into cortical organoids. Two additional tumor replicates are shown pre- and post-transplantation. In each case, a graph of the most highly correlated broad cell type for each single-cell cluster is depicted as parts of a whole. Like in SF12011 (shown in FIG. 4), PTPRZ1 positive cells can give rise to cell types that do not exist in the initial population, particularly astrocytes and neuronal populations.

FIG. 12B. Transplants of primary glioblastoma tumors into cortical organoids. Immunofluorescence of scrambled control hairpin and short hairpin targeting PTPRZ1 validates protein knockdown. Western blot and qPCR to validate knockdown (quantified across 3 replicates for qPCR) were also performed.

FIG. 12C. Transplants of primary glioblastoma tumors into cortical organoids. Short hairpin induced knockdown of PTPRZ1 and its ligand PTN significantly (PTPRZ1 **** = p < 0.0001, PTN * = p < 0.05, Student’s two-sided t-test) decrease the length of somal translocation length using 3 biological replicates and 4 technical replicates each in primary slice culture.

FIG. 12D. Transplants of primary glioblastoma tumors into cortical organoids. Proliferation as indicated by cell titer blue assay is shown for PTPRZ1 shScr (Ctrl) or shPTPRZ1 (KD). The difference is not significant at any time point, n=5. On the right, an invasion well with white lines added to mark the bounds of the invasion chamber. Immunofluorescence shows the higher percentage of cells that are PTRPZ1 positive compared to IBA1.

FIG. 12E. Transplants of primary glioblastoma tumors into cortical organoids. Immunofluorescence marking proliferating tumor cells (Ki67 for proliferation, Luciferase for tumor cells) in the DBTRGFL F2 tumor sample. PTPRZ1 positive proliferating cells are also stained and quantification using Imaris is shown in the graph on the left.

### BRIEF DESCRIPTION OF SEQUENCES

SEQ ID NO: 1: Protein sequence of human PTPRZ1 (GenBank ID: AAA60225.1)

MRILKRFLACIQLLCVCRLDWANGYYRQQRKLVEEIGWSYTGALNQKNWG
KKYPTCNSPKQSPINIDEDLTQVNVNLKKLKFQGWDKTSLENTFIHNTGK
TVEINLTNDYRVSGGVSEMVFKASKITFHWGKCNMSSDGSEHSLEGQKFP
LEMQIYCFDADRFSSFEEAVKGKGKLRALSILFEVGTEENLDFKAIIDGV
ESVSRFGKQAALDPFILLNLLPNSTDKYYIYNGSLTS P PCTDTVDWIV
FKDTVSISESQLAVFCEVLTMQQSGYVMLMDYLQNNFREQQYKFSRQVFS
SYTGKEEIHEAVCSSEPENVQADPENYTSLLVTWERPRVVYDTMIEKFAV
LYQQLDGEDQTKHEFLTDGYQDLGAILNNLLPNMSYVLQIVAICTNGLYG
KYSDQLIVDMPTDNPELDLFPELIGTEEIIKEEEEGKDIEEGAIVNPGRD
SATNQIRKKEPQISTTTHYNRIGTKYNEAKTNRSPTRGSEFSGKGDVPNT
SLNSTSQPVTKLATEKDISLTSQTVTELPPHTVEGTSASLNDGSKTVLRS
PHMNLSGTAESLNTVSITEYEEESLLTSFKLDTGAEDSSGSSPATSAIPF
ISENISQGYIFSSENPETITYDVLIPESARNASEDSTSSGSEESLKDPSM
EGNVWFPSSTDITAQPDVGSGRESFLQTNYTEIRVDESEKTTKSFSAGPV
MSQGPSVTDLEMPHYSTFAYFPTEVTPHAFTPSSRQQDLVSTVNVVYSQT
TQPVYNGETPLQPSYSSEVFPLVTPLLLDNQILNTTPAASSSDSALHATP
VFPSVDVSFESILSSYDGAPLLPFSSASFSSELFRHLHTVSQILPQVTSA
TESDKVPLHASLPVAGGDLLLEPSLAQYSDVLSTTHAASETLEFGSESGV
LYKTLMFSQVEPPSSDAMMHARSSGPEPSYALSDNEGSQHIFTVSYSSAI
PVHDSVGVTYQGSLFSGPSHIPIPKSSLITPTASLLQPTHALSGDGEWSG
ASSDSEFLLPDTDGLTALNISSPVSVAEFTYTTSVFGDDNKALSKSEIIY
GNETELQIPSFNEMVYPSESTVMPNMYDNVNKLNASLQETSVSISSTKGM
FPGSLAHTTTKVFDHEISQVPENNFSVQPTHTVSQASGDTSLKPVLSANS
EPASSDPASSEMLSPSTQLLFYETSASFSTEVLLQPSFQASDVDTLLKTV
LPAVPSDPILVETPKVDKISSTMLHLIVSNSASSENMLHSTSVPVFDVSP
TSHMHSASLQGLTISYASEKYEPVLLKSESSHQVVPSLYSNDELFQTANL
EINQAHPPKGRHVFATPVLSIDEPLNTLINKLIHSDEILTSTKSSVTGKV
FAGIPTVASDTFVSTDHSVPIGNGHVAITAVSPHRDGSVTSTKLLFPSKA
TSELSHSAKSDAGLVGGGEDGDTDDDGDDDDDRDSDGLSIHKCMSCSSYR
ESQEKVMNDSDTHENSLMDQNNPISYSLSENSEEDNRVTSVSSDSQTGMD
RSPGKSPSANGLSQKHNDGKEENDIQTGSALLPLSPESKAWAVLTSDEES
GSGQGTSDSLNENETSTDFSFADTNEKDADGILAAGDSEITPGFPQSPTS
SVTSENSEVFHVSEAEASNSSHESRIGLAEGLESEKKAVIPLVIVSALTF
ICLVVLVGILIYWRKCFQTAHFYLEDSTSPRVISTPPTPIFPISDDVGAI
PIKHFPKHVADLHASSGFTEEFETLKEFYQEVQSCTVDLGITADSSNHPD
NKHKNRYINIVAYDHSRVKLAQLAEKDGKLTDYINANYVDGYNRPKAYIA
AQGPLKSTAEDFWRMIWEHNVEVIVMITNLVEKGRRKCDQYWPADGSEEY
GNFLVTQKSVQVLAYYTVRNFTLRNTKIKKGSQKGRPSGRVVTQYHYTQW
PDMGVPEYSLPVLTFVRKAAYAKRHAVGPVVVHCSAGVGRTGTYIVLDSM
LQQIQHEGTVNIFGFLKHIRSQRNYLVQTEEQYVFIHDTLVEAILSKETE
VLDSHIHAYVNALLIPGPAGKTKLEKQFQLLSQSNIQQSDYSAALKQCNR
EKNRTSSIIPVERSRVGISSLSGEGTDYINASYIMGYYQSNEFIITQHPL
LHTIKDFWRMIWDHNAQLVVMIPDGQNMAEDEFVYWPNKDEPINCESFKV
TLMAEEHKCLSNEEKLIIQDFILEATQDDYVLEVRHFQCPKWPNPDSPIS
KTFELISVIKEEAANRDGPMIVHDEHGGVTAGTFCALTTLMHQLEKENSV
DVYQVAKMINLMRPGVFADIEQYQFLYKVILSLVSTRQEENPSTSLDSNG
AALPDGNIAESLESLV

SEQ ID NO: 2: mRNA sequence of human PTPRZ1 (GenBank ID: M93426.1)

CACACATACGCACGCACGATCTCACTTCGATCTATACACTGGAGGATTAA
AACAAACAAACAAAAAAAACATTTCCTTCGCTCCCCCTCCCTCTCCACTC
TGAGAAGCAGAGGAGCCGCACGGCGAGGGGCCGCAGACCGTCTGGAAATG
CGAATCCTAAAGCGTTTCCTCGCTTGCATTCAGCTCCTCTGTGTTTGCCG
CCTGGATTGGGCTAATGGATACTACAGACAACAGAGAAAACTTGTTGAAG
AGATTGGCTGGTCCTATACAGGAGCACTGAATCAAAAAAATTGGGGAAAG
AAATATCCAACATGTAATAGCCCAAAACAATCTCCTATCAATATTGATGA
AGATCTTACACAAGTAAATGTGAATCTTAAGAAACTTAAATTTCAGGGTT
GGGATAAAACATCATTGGAAAACACATTCATTCATAACACTGGGAAAACA
GTGGAAATTAATCTCACTAATGACTACCGTGTCAGCGGAGGAGTTTCAGA
AATGGTGTTTAAAGCAAGCAAGATAACTTTTCACTGGGGAAAATGCAATA
TGTCATCTGATGGATCAGAGCATAGTTTAGAAGGACAAAAATTTCCACTT
GAGATGCAAATCTACTGCTTTGATGCGGACCGATTTTCAAGTTTTGAGGA
AGCAGTCAAAGGAAAAGGGAAGTTAAGAGCTTTATCCATTTTGTTTGAGG
TTGGGACAGAAGAAAATTTGGATTTCAAAGCGATTATTGATGGAGTCGAA
AGTGTTAGTCGTTTTGGGAAGCAGGCTGCTTTAGATCCATTCATACTGTT
GAACCTTCTGCCAAACTCAACTGACAAGTATTACATTTACAATGGCTCAT
TGACATCTCCTCCCTGCACAGACACAGTTGACTGGATTGTTTTTAAAGAT
ACAGTTAGCATCTCTGAAAGCCAGTTGGCTGTTTTTTGTGAAGTTCTTAC
AATGCAACAATCTGGTTATGTCATGCTGATGGACTACTTACAAAACAATT
TTCGAGAGCAACAGTACAAGTTCTCTAGACAGGTGTTTTCCTCATACACT
GGAAAGGAAGAGATTCATGAAGCAGTTTGTAGTTCAGAACCAGAAAATGT
TCAGGCTGACCCAGAGAATTATACCAGCCTTCTTGTTACATGGGAAAGAC
CTCGAGTCGTTTATGATACCATGATTGAGAAGTTTGCAGTTTTGTACCAG
CAGTTGGATGGAGAGGACCAAACCAAGCATGAATTTTTGACAGATGGCTA
TCAAGACTTGGGTGCTATTCTCAATAATTTGCTACCCAATATGAGTTATG
TTCTTCAGATAGTAGCCATATGCACTAATGGCTTATATGGAAAATACAGC
GACCAACTGATTGTCGACATGCCTACTGATAATCCTGAACTTGATCTTTT
CCCTGAATTAATTGGAACTGAAGAAATAATCAAGGAGGAGGAAGAGGGAA
AAGACATTGAAGAAGGCGCTATTGTGAATCCTGGTAGAGACAGTGCTACA
AACCAAATCAGGAAAAAGGAACCCCAGATTTCTACCACAACACACTACAA
TCGCATAGGGACGAAATACAATGAAGCCAAGACTAACCGATCCCCAACAA
GAGGAAGTGAATTCTCTGGAAAGGGTGATGTTCCCAATACATCTTTAAAT
TCCACTTCCCAACCAGTCACTAAATTAGCCACAGAAAAAGATATTTCCTT
GACTTCTCAGACTGTGACTGAACTGCCACCTCACACTGTGGAAGGTACTT
CAGCCTCTTTAAATGATGGCTCTAAAACTGTTCTTAGATCTCCACATATG
AACTTGTCGGGGACTGCAGAATCCTTAAATACAGTTTCTATAACAGAATA
TGAGGAGGAGAGTTTATTGACCAGTTTCAAGCTTGATACTGGAGCTGAAG
ATTCTTCAGGCTCCAGTCCCGCAACTTCTGCTATCCCATTCATCTCTGAG
AACATATCCCAAGGGTATATATTTTCCTCCGAAAACCCAGAGACAATAAC
ATATGATGTCCTTATACCAGAATCTGCTAGAAATGCTTCCGAAGATTCAA
CTTCATCAGGTTCAGAAGAATCACTAAAGGATCCTTCTATGGAGGGAAAT
GTGTGGTTTCCTAGCTCTACAGACATAACAGCACAGCCCGATGTTGGATC
AGGCAGAGAGAGCTTTCTCCAGACTAATTACACTGAGATACGTGTTGATG
AATCTGAGAAGACAACCAAGTCCTTTTCTGCAGGCCCAGTGATGTCACAG
GGTCCCTCAGTTACAGATCTGGAAATGCCACATTATTCTACCTTTGCCTA
CTTCCCAACTGAGGTAACACCTCATGCTTTTACCCCATCCTCCAGACAAC
AGGATTTGGTCTCCACGGTCAACGTGGTATACTCGCAGACAACCCAACCG
GTATACAATGGTGAGACACCTCTTCAACCTTCCTACAGTAGTGAAGTCTT
TCCTCTAGTCACCCCTTTGTTGCTTGACAATCAGATCCTCAACACTACCC
CTGCTGCTTCAAGTAGTGATTCGGCCTTGCATGCTACGCCTGTATTTCCC
AGTGTCGATGTGTCATTTGAATCCATCCTGTCTTCCTATGATGGTGCACC
TTTGCTTCCATTTTCCTCTGCTTCCTTCAGTAGTGAATTGTTTCGCCATC
TGCATACAGTTTCTCAAATCCTTCCACAAGTTACTTCAGCTACCGAGAGT
GATAAGGTGCCCTTGCATGCTTCTCTGCCAGTGGCTGGGGGTGATTTGCT
ATTAGAGCCCAGCCTTGCTCAGTATTCTGATGTGCTGTCCACTACTCATG
CTGCTTCAGAGACGCTGGAATTTGGTAGTGAATCTGGTGTTCTTTATAAA
ACGCTTATGTTTTCTCAAGTTGAACCACCCAGCAGTGATGCCATGATGCA
TGCACGTTCTTCAGGGCCTGAACCTTCTTATGCCTTGTCTGATAATGAGG
GCTCCCAACACATCTTCACTGTTTCTTACAGTTCTGCAATACCTGTGCAT
GATTCTGTGGGTGTAACTTATCAGGGTTCCTTATTTAGCGGCCCTAGCCA
TATACCAATACCTAAGTCTTCGTTAATAACCCCAACTGCATCATTACTGC
AGCCTACTCATGCCCTCTCTGGTGATGGGGAATGGTCTGGAGCCTCTTCT
GATAGTGAATTTCTTTTACCTGACACAGATGGGCTGACAGCCCTTAACAT
TTCTTCACCTGTTTCTGTAGCTGAATTTACATATACAACATCTGTGTTTG
GTGATGATAATAAGGCGCTTTCTAAAAGTGAAATAATATATGGAAATGAG
ACTGAACTGCAAATTCCTTCTTTCAATGAGATGGTTTACCCTTCTGAAAG
CACAGTCATGCCCAACATGTATGATAATGTAAATAAGTTGAATGCGTCTT
TACAAGAAACCTCTGTTTCCATTTCTAGCACCAAGGGCATGTTTCCAGGG
TCCCTTGCTCATACCACCACTAAGGTTTTTGATCATGAGATTAGTCAAGT
TCCAGAAAATAACTTTTCAGTTCAACCTACACATACTGTCTCTCAAGCAT
CTGGTGACACTTCGCTTAAACCTGTGCTTAGTGCAAACTCAGAGCCAGCA
TCCTCTGACCCTGCTTCTAGTGAAATGTTATCTCCTTCAACTCAGCTCTT
ATTTTATGAGACCTCAGCTTCTTTTAGTACTGAAGTATTGCTACAACCTT
CCTTTCAGGCTTCTGATGTTGACACCTTGCTTAAAACTGTTCTTCCAGCT
GTGCCCAGTGATCCAATATTGGTTGAAACCCCCAAAGTTGATAAAATTAG
TTCTACAATGTTGCATCTCATTGTATCAAATTCTGCTTCAAGTGAAAACA
TGCTGCACTCTACATCTGTACCAGTTTTTGATGTGTCGCCTACTTCTCAT
ATGCACTCTGCTTCACTTCAAGGTTTGACCATTTCCTATGCAAGTGAGAA
ATATGAACCAGTTTTGTTAAAAAGTGAAAGTTCCCACCAAGTGGTACCTT
CTTTGTACAGTAATGATGAGTTGTTCCAAACGGCCAATTTGGAGATTAAC
CAGGCCCATCCCCCAAAAGGAAGGCATGTATTTGCTACACCTGTTTTATC
AATTGATGAACCATTAAATACACTAATAAATAAGCTTATACATTCCGATG
AAATTTTAACCTCCACCAAAAGTTCTGTTACTGGTAAGGTATTTGCTGGT
ATTCCAACAGTTGCTTCTGATACATTTGTATCTACTGATCATTCTGTTCC
TATAGGAAATGGGCATGTTGCCATTACAGCTGTTTCTCCCCACAGAGATG
GTTCTGTAACCTCAACAAAGTTGCTGTTTCCTTCTAAGGCAACTTCTGAG
CTGAGTCATAGTGCCAAATCTGATGCCGGTTTAGTGGGTGGTGGTGAAGA
TGGTGACACTGATGATGATGGTGATGATGATGATGACAGAGATAGTGATG
GCTTATCCATTCATAAGTGTATGTCATGCTCATCCTATAGAGAATCACAG
GAAAAGGTAATGAATGATTCAGACACCCACGAAAACAGTCTTATGGATCA
GAATAATCCAATCTCATACTCACTATCTGAGAATTCTGAAGAAGATAATA
GAGTCACAAGTGTATCCTCAGACAGTCAAACTGGTATGGACAGAAGTCCT
GGTAAATCACCATCAGCAAATGGGCTATCCCAAAAGCACAATGATGGAAA
AGAGGAAAATGACATTCAGACTGGTAGTGCTCTGCTTCCTCTCAGCCCTG
AATCTAAAGCATGGGCAGTTCTGACAAGTGATGAAGAAAGTGGATCAGGG
CAAGGTACCTCAGATAGCCTTAATGAGAATGAGACTTCCACAGATTTCAG
TTTTGCAGACACTAATGAAAAAGATGCTGATGGGATCCTGGCAGCAGGTG
ACTCAGAAATAACTCCTGGATTCCCACAGTCCCCAACATCATCTGTTACT
AGCGAGAACTCAGAAGTGTTCCACGTTTCAGAGGCAGAGGCCAGTAATAG
TAGCCATGAGTCTCGTATTGGTCTAGCTGAGGGGTTGGAATCCGAGAAGA
AGGCAGTTATACCCCTTGTGATCGTGTCAGCCCTGACTTTTATCTGTCTA
GTGGTTCTTGTGGGTATTCTCATCTACTGGAGGAAATGCTTCCAGACTGC
ACACTTTTACTTAGAGGACAGTACATCCCCTAGAGTTATATCCACACCTC
CAACACCTATCTTTCCAATTTCAGATGATGTCGGAGCAATTCCAATAAAG
CACTTTCCAAAGCATGTTGCAGATTTACATGCAAGTAGTGGGTTTACTGA
AGAATTTGAGACACTGAAAGAGTTTTACCAGGAAGTGCAGAGCTGTACTG
TTGACTTAGGTATTACAGCAGACAGCTCCAACCACCCAGACAACAAGCAC
AAGAATCGATACATAAATATCGTTGCCTATGATCATAGCAGGGTTAAGCT
AGCACAGCTTGCTGAAAAGGATGGCAAACTGACTGATTATATCAATGCCA
ATTATGTTGATGGCTACAACAGACCAAAAGCTTATATTGCTGCCCAAGGC
CCACTGAAATCCACAGCTGAAGATTTCTGGAGAATGATATGGGAACATAA
TGTGGAAGTTATTGTCATGATAACAAACCTCGTGGAGAAAGGAAGGAGAA
AATGTGATCAGTACTGGCCTGCCGATGGGAGTGAGGAGTACGGGAACTTT
CTGGTCACTCAGAAGAGTGTGCAAGTGCTTGCCTATTATACTGTGAGGAA
TTTTACTCTAAGAAACACAAAAATAAAAAAGGGCTCCCAGAAAGGAAGAC
CCAGTGGACGTGTGGTCACACAGTATCACTACACGCAGTGGCCTGACATG
GGAGTACCAGAGTACTCCCTGCCAGTGCTGACCTTTGTGAGAAAGGCAGC
CTATGCCAAGCGCCATGCAGTGGGGCCTGTTGTCGTCCACTGCAGTGCTG
GAGTTGGAAGAACAGGCACATATATTGTGCTAGACAGTATGTTGCAGCAG
ATTCAACACGAAGGAACTGTCAACATATTTGGCTTCTTAAAACACATCCG
TTCACAAAGAAATTATTTGGTACAAACTGAGGAGCAATATGTCTTCATTC
ATGATACACTGGTTGAGGCCATACTTAGTAAAGAAACTGAGGTGCTGGAC
AGTCATATTCATGCCTATGTTAATGCACTCCTCATTCCTGGACCAGCAGG
CAAAACAAAGCTAGAGAAACAATTCCAGCTCCTGAGCCAGTCAAATATAC
AGCAGAGTGACTATTCTGCAGCCCTAAAGCAATGCAACAGGGAAAAGAAT
CGAACTTCTTCTATCATCCCTGTGGAAAGATCAAGGGTTGGCATTTCATC
CCTGAGTGGAGAAGGCACAGACTACATCAATGCCTCCTATATCATGGGCT
ATTACCAGAGCAATGAATTCATCATTACCCAGCACCCTCTCCTTCATACC
ATCAAGGATTTCTGGAGGATGATATGGGACCATAATGCCCAACTGGTGGT
TATGATTCCTGATGGCCAAAACATGGCAGAAGATGAATTTGTTTACTGGC
CAAATAAAGATGAGCCTATAAATTGTGAGAGCTTTAAGGTCACTCTTATG
GCTGAAGAACACAAATGTCTATCTAATGAGGAAAAACTTATAATTCAGGA
CTTTATCTTAGAAGCTACACAGGATGATTATGTACTTGAAGTGAGGCACT
TTCAGTGTCCTAAATGGCCAAATCCAGATAGCCCCATTAGTAAAACTTTT
GAACTTATAAGTGTTATAAAAGAAGAAGCTGCCAATAGGGATGGGCCTAT
GATTGTTCATGATGAGCATGGAGGAGTGACGGCAGGAACTTTCTGTGCTC
TGACAACCCTTATGCACCAACTAGAAAAAGAAAATTCCGTGGATGTTTAC
CAGGTAGCCAAGATGATCAATCTGATGAGGCCAGGAGTCTTTGCTGACAT
TGAGCAGTATCAGTTTCTCTACAAAGTGATCCTCAGCCTTGTGAGCACAA
GGCAGGAAGAGAATCCATCCACCTCTCTGGACAGTAATGGTGCAGCATTG
CCTGATGGAAATATAGCTGAGAGCTTAGAGTCTTTAGTTTAACACAGAAA
GGGGTGGGGGGACTCACATCTGAGCATTGTTTTCCTCTTCCTAAAATTAG
GCAGGAAAATCAGTCTAGTTCTGTTATCTGTTGATTTCCCATCACCTGAC
AGTAACTTTCATGACATAGGATTCTGCCGCCAAATTTATATCATTAACAA
TGTGTGCCTTTTTGCAAGACTTGTAATTTACTTATTATGTTTGAACTAAA
ATGATTGAATTTTACAGTATTTCTAAGAATGGAATTGTGGTATTTTTTTC
TGTATTGATTTTAACAGAAAATTTCAATTTATAGAGGTTAGGAATTCCAA
ACTACAGAAAATGTTTGTTTTTAGTGTCAAATTTTTAGCTGTATTTGTAG
CAATTATCAGGTTTGCTAGAAATATAACTTTTAATACAGTAGCCTGTAAA
TAAAACACTCTTCCATATGATATTCAACATTTTACAACTGCAGTATTCAC
CTAAAGTAGAAATAATCTGTTACTTATTGTAAATACTGCCCTAGTGTCTC
CATGGACCAAATTTATATTTATAATTGTAGATTTTTATATTTTACTACTG
AGTCAAGTTTTCTAGTTCTGTGTAATTGTTTAGTTTAATGACGTAGTTCA
TTAGCTGGTCTTACTCTACCAGTTTTCTGACATTGTATTGTGTTACCTAA
GTCATTAACTTTGTTTCAGCATGTAATTTTAACTTTTGTGGAAAATAGAA
ATACCTTCATTTTGAAAGAAGTTTTTATGAGAATAACACCTTACCAAACA
TTGTTCAAATGGTTTTTATCCAAGGAATTGCAAAAATAAATATAAATATT
GCCATTAAAAAAAAAAAAAAAAAAAAAAAAAAAAAAAAAAA

SEQ ID NO: 3: Protein sequence of human PTN (NCBI Reference Sequence: NP_002816.1)

MQAQQYQQQRRKFAAAFLAFIFILAAVDTAEAGKKEKPEKKVKKSDCGEW
QWSVCVPTSGDCGLGTREGTRTGAECKQTMKTQRCKIPCNWKKQFGAECK
YQFQAWGECDLNTALKTRTGSLKRALHNAECQKTVTISKPCGKLTKPKPQ
AESKKKKKEGKKQEKMLD

SEQ ID NO: 4: mRNA sequence of human PTN (NCBI Reference Sequence: NM_002825.7)

ATTCTCCATTTCCCTTCCGTTCCCTCCCTGTCAGGGCGTAATTGAGTCAA
AGGCAGGATCAGGTTCCCCGCCTTCCAGTCCAAAAATCCCGCCAAGAGAG
CCCCAGAGCAGAGGAAAATCCAAAGTGGAGAGAGGGGAAGAAAGAGACCA
GTGAGTCATCCGTCCAGAAGGCGGGGAGAGCAGCAGCGGCCCAAGCAGGA
GCTGCAGCGAGCCGGGTACCTGGACTCAGCGGTAGCAACCTCGCCCCTTG
CAACAAAGGCAGACTGAGCGCCAGAGAGGACGTTTCCAACTCAAAAATGC
AGGCTCAACAGTACCAGCAGCAGCGTCGAAAATTTGCAGCTGCCTTCTTG
GCATTCATTTTCATACTGGCAGCTGTGGATACTGCTGAAGCAGGGAAGAA
AGAGAAACCAGAAAAAAAAGTGAAGAAGTCTGACTGTGGAGAATGGCAGT
GGAGTGTGTGTGTGCCCACCAGTGGAGACTGTGGGCTGGGCACACGGGAG
GGCACTCGGACTGGAGCTGAGTGCAAGCAAACCATGAAGACCCAGAGATG
TAAGATCCCCTGCAACTGGAAGAAGCAATTTGGCGCGGAGTGCAAATACC
AGTTCCAGGCCTGGGGAGAATGTGACCTGAACACAGCCCTGAAGACCAGA
ACTGGAAGTCTGAAGCGAGCCCTGCACAATGCCGAATGCCAGAAGACTGT
CACCATCTCCAAGCCCTGTGGCAAACTGACCAAGCCCAAACCTCAAGCAG
AATCTAAGAAGAAGAAAAAGGAAGGCAAGAAACAGGAGAAGATGCTGGAT
TAAAAGATGTCACCTGTGGAACATAAAAAGGACATCAGCAAACAGGATCA
GTTAACTATTGCATTTATATGTACCGTAGGCTTTGTATTCAAAAATTATC
TATAGCTAAGTACACAATAAGCAAAAACAAAAAGAAAAGAAAATTTTTGT
AGTAGCGTTTTTTAAATGTATACTATAGTACCAGTAGGGGCTTATAATAA
AGGACTGTAATCTTATTTAGGAAGTTGACTTATAGTACATGATAAATGAT
AGACAATTGAGGTAAGTTTTTTGAAATTATGTGACATTTTACATTAAATT
TTTTTTACATTTTTTGGGCAGCAATTTAAATGTTATGACTATGTAAACTA
CTTCTCTTGTTAGGTAATTTTTTTCACCTAGATTTTTTTCCCAATTGAGA
AAAATATATACTAAACAAAATAGCAATAAAACATAATCACTCTATTTGAA
GAAAATATCTTGTTTTCTGCCAATAGATTTTTTAAAATGTAGTCAGCAAA
ATGGGGGTGGGGAAGCAGAGCATGTCCTAGTTCAATGTTGACTTTTTTTT
TTTTTAAAGAAAAGCATTAAGACATAAAATTCTTTCACTTTGGCAGAAGC
ATTTGTTTTCTTGATGAAATTATTTTTCCATCTGAGGAAAAAAATACTAG
GAAAATAAATCAAGGTGATGCTGAAAAAAAAA

SEQ ID NO: 5: The sequence of an exemplary shRNA for PTPRZ1.

CCGGCCAGACAACAAGCACAAGAATCTCGAGATTCTTGTGCTTGTTGTCT
GGTTTTT

SEQ ID NO: 6: The sequence of an exemplary shRNA for PTN.

CCGGAGGCAAGAAACAGGAGAAGATCTCGAGATCTTCTCCTGTTTCTTGC
CTTTTTT

### DETAILED DESCRIPTION

As used herein, the singular forms “a,” “an,” and “the” are intended to include the plural forms as well, unless the context clearly indicates otherwise. To the extent that the terms “including,” “includes,” “having,” “has,” “with,” or variants thereof are used in either the detailed description and/or the claims, such terms are intended to be inclusive in a manner similar to the term “comprising.” The phrase “consisting essentially of” or “consists essentially of” indicates that the claim encompasses embodiments containing the specified materials or steps and those that do not materially affect the basic and novel characteristics of the claim. The transitional terms/phrases (and any grammatical variations thereof) “comprising,” “comprises,” “comprise,” “consisting essentially of,” “consists essentially of,” “consisting,” and “consists” can be used interchangeably.

The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with an inhibitor of an PTPRZ1 pathway, its use in the pharmaceutical compositions of the invention is contemplated.

When ranges are used herein, such as for dose ranges, combinations and subcombinations of ranges (e.g., subranges within the disclosed range), specific embodiments therein are intended to be explicitly included.

“Subject” refers to an animal, such as a mammal, for example a human. The methods described herein can be useful in both humans and non-human animals. In some embodiments, the subject is a mammal (such as an animal model of disease), and in some embodiments, the subject is human.

Glioblastoma is a devastating form of brain cancer. To identify aspects of tumor heterogeneity that may illuminate drivers of tumor invasion, a glioblastoma tumor cell atlas was created with single-cell transcriptomics of cancer cells mapped onto a reference framework of the developing and adult human brain. Multiple glioblastoma stem cell (GSC) subtypes exist within a single tumor. Within these GSCs, an invasive cell population was identified, which is similar to the outer radial glia (oRG), a fetal cell type that expands the stem cell niche in normal human cortical. Using live time-lapse imaging of primary resected tumors, tumor derived oRG-like cells were identified to undergo characteristic mitotic somal translocation (MST) behavior previously only observed in human development, suggesting a reactivation of developmental programs. In addition, PTPRZ1 is shown to mediate both MST and glioblastoma tumor invasion.

PTPRZ1 is a member of the PTPR family. PTPRZ1 has two cytoplasmic tyrosine phosphatase domains, an alpha-carbonic anhydrase domain, chondroitin sulfate proteoglycans and a fibronectin type-III domain. A secreted growth factor pleiotrophin (PTN) interacts PTPRZ1 and inactivates the phosphatase activity of PTPRZ1. Such inactivation increases tyrosine phosphorylation status of other signaling molecules such as β-catenin, Fyn and RhoGAP. Exemplary sequences of human PTPRZ1 protein and mRNA as well as human PTN protein and mRNA are provided in SEQ ID NOs: 1 to 4.

Thus, the role of PTPRZ1 in invasion of a cancer of a nervous system, particularly, glioblastoma, is described. Also, based on the role of PTPRZ1 in the invasion of a cancer, methods are described for inhibiting the invasion of a cancer of a nervous system, such as brain cancer, particularly, glioblastoma.

The phrase “invasion of a cancer” refers to dissemination of cancer cells into surrounding healthy tissue that causes spread of the cancer into the healthy tissue. As described herein, specific cancer cells within a cancer of a nervous system spread outside the tumor into the surrounding healthy tissue, potentially, via a process similar to “mitotic somal translocation.” Mitotic somal translation is displayed by oRG cells and is a migratory behavior where the soma translocates towards the cortical plate before cytokinesis. MST is believed to be involved during brain development for germinal zone expansion.

To address the issues presented by the conventional methods of culturing cancer cells in genetically modified organoids, certain embodiments of the invention provide a brain organoid generated from an ESC or an iPSC, the organoid further comprising implanted therein a cancer cell derived from a cancer of a nervous system, wherein the ESC or iPSC is not genetically modified to render it oncogenic. The ESC or iPSC can be a human ESC or human iPSC.

In preferred embodiments, the cancer cell is a primary cancer cell. The phrase “primary cancer cell” as used herein refers to a cancer cell that is isolated from a cancer of a subject, for example, a tumor sample from a subject, and is cultured in a manner that maintains the cell’s viability and may allow the cell to grow but does not allow the cell to divide to a significant extent. Accordingly, when a cell is implanted in a brain organoid, it has not significantly divided since being removed from the subject. For example, the cancer cell may not have been cultured for a significant period of time. In certain aspects, the cancer cell may be isolated by dissociating a tumor sample in a suitable culture medium and implanting the dissociated cells. The tumor sample may be fresh or previously frozen. The dissociated cells may be implanted immediately or may be subjected to a selection step for isolating cells positive for a marker, such as, PTPRZ1 or PTN. After implantation into the brain organoid, the cancer cell can grow and divide. When implanted into a brain organoid, a primary cancer cell can remain viable for at least one week (e.g., at least two weeks, at least three weeks, at least four weeks, or at least 60 days) . Such cultures find use in screening of agents that inhibit growth of cancers and/or inhibit expression of proteins that are required for rendering cancer cells invasive. For example, such cultures may be used for screening for agents that decrease expression of PTPRZ1 or PTN. Agents of interest that may be used for such screens include small molecules, nucleic acids, peptides, and proteins, such as antibodies.

The primary cancer cell can be isolated from a cancer selected from a brain stem glioma, pineal astrocytic tumor, pilocytic astrocytoma, diffuse astrocytoma, anaplastic astrocytoma, glioblastoma, oligodendroglial tumor, mixed glioma, ependymal tumor, medulloblastoma, pineal parenchymal tumor, meningeal tumor, germ cell tumor, and craniopharyngioma. In certain aspects, the primary cancer cell may be a PTPRZ1-positive cancer cell.

The brain organoid can be a neurosphere, neural aggregate, neural rossette, cortical spheroid, cortical organoid, cerebral organoid, or whole-brain organoid.

The brain organoid may be a cortical organoid generated by culturing untransformed hESCs or iPSCs in a medium comprising a Rho kinase inhibitor, a Wnt signal inhibitor and a TGFβ signal inhibitor on a low adhesion substrate to generate aggregates. Culturing can be carried out for a period of time sufficient to generate the aggregates. The method for generating cortical organoid further comprises culturing the aggregates in a medium comprising a Wnt signal inhibitor and a TGFβ signal inhibitor and lacking a Rho kinase inhibitor, on a low adhesion substrate to generate the cortical organoid. Culturing can be carried out for a period of time sufficient to generate the cortical organoid.

In certain aspects, the cortical organoid expresses one or more of the telencephalon markers Foxg1 and Six 3. The Wnt signal inhibitor may be IWR-1. The TGFβ signal inhibitor may be SB431542. The Rho kinase inhibitor may be Y-27632.

In certain aspects, the culturing methods disclosed herein may be used for screening a candidate agent for activity in reducing cancer cell growth and/or invasion, the method comprising: culturing the primary cancer cells in the brain organoid in the presence of the candidate agent and culturing the primary cancer cells in the brain organoid. Reduced cancer cell growth of and/or invasion as compared to the cancer cell growth and/or invasion, respectively, in absence of the candidate agent identifies the candidate agent as having activity in reducing cancer cell growth and/or invasion.

The reduction in cancer cell growth may be a 5% reduction or more (e.g., a 10%, 20%, 30%, 40%, 50%, or more) compared to the growth in absence of the candidate agent. In certain aspects, growth may be measured by tumor volume.

The reduction in cancer cell invasion may be determined by measuring spread of cancer cells outside the initial implantation site.

The present disclosure also provides a method of inhibiting the invasion of a cancer of the nervous system in a subject suffering from the cancer of the nervous system, the method comprising administering to the subject an inhibitor of PTPRZ1 pathway. The inhibitor of the PTPRZ1 pathway is administered in an amount effective to inhibit the invasion of cancer.

The cancer inhibited according to the methods disclosed herein is a cancer of the nervous system. Such cancers affect the brain, spinal cord, a part of the neuroendocrine system, such as a neuroendocrine gland, and include brain stem glioma, pineal astrocytic tumor, pilocytic astrocytomas, diffuse astrocytomas, anaplastic astrocytoma, glioblastoma, oligodendroglial tumor, mixed glioma, ependymal tumor, medulloblastomas, pineal parenchymal tumor, meningeal tumors, germ cell tumors, and craniopharyngiomas.

The inhibitor of the PTPRZ1 pathway can be an inhibitor of the expression or activity of PTPRZ1. An inhibitor of PTPRZ1 expression can be an inhibitory oligonucleotide that specifically inhibits the transcription or translation of PTPRZ1 mRNA or protein.

An inhibitor of mRNA transcription can be an oligonucleotide that blocks or decreases the expression of PTPRZ1 or PTN gene by targeting the nucleic acids encoding the PTPRZ1 or PTN protein. Methods are known to a person of ordinary skill in the art for the preparation of oligonucleotides that would specifically inhibit the expression of PTPRZ1 or PTN without affecting expression of other genes, for example, based on the genomic or mRNA sequences of PTPRZ1 or PTN. The antisense oligonucleotide can contain about 10 to about 100 nucleotides, preferably, about 15 to about 50 nucleotides, even more preferably, about 18 to about 25 nucleotides, and most preferably, about 21 nucleotides. In certain embodiments, the oligonucleotides can comprise chemical modifications to increase nuclease resistance, such as phosphorothioate linkages and 2′-O-sugar modifications. Additional such modifications are also known to those of ordinary skill in the art and are within the purview of the invention.

Oligonucleotides inhibiting the translation are typically called interfering RNA (RNAi). Accordingly, an oligonucleotide inhibitor of PTPRZ1 expression can be an RNAi. An RNAi can down-regulate the expression of PTPRZ1 and includes small interfering RNA (siRNA), double-stranded RNA (dsRNA), single-stranded RNA (ssRNA), micro-RNA (miRNA), and short hairpin RNA (shRNA). A person of ordinary skill can design an appropriate RNAi for inhibiting the translation of PTPRZ1 or PTN protein, for example, based on the sequence of the PTPRZ1 or PTN mRNA. In certain embodiments, the oligonucleotides are administered to a subject in the form of a nucleotide vector that encodes for the RNAi.

In one embodiment, an RNAi inhibits the expression of PTPRZ1 protein, and is an shRNA comprising the sequence of SEQ ID NO: 5. In another embodiment, an RNAi inhibits the expression of PTN protein, and is an shRNA comprising the sequence of SEQ ID NO: 6.

An inhibitor of activity of PTPRZ1 or PTN protein can be an antibody or an antigen binding fragment of an antibody that specifically binds to PTPRZ1 or PTN. In one embodiment, the anti-PTPRZ1 antibody has the CDRs of the anti-PTPRZ1 antibody available from Santa Cruz™ catalogue number sc-33664.

Santa Cruz anti-PTPRZ1 antibody catalogue number sc-33664 may be used, and have been tested to purify the PTPRZ1 expressing cancer cells. No anti-PTN antibodies have been tested as part of this work.

An antibody can be a polypeptide that specifically binds to an antigen. Typically, an antibody is a glycoprotein comprising at least two heavy (H) chains and two light (L) chains connected by disulfide bonds. Each heavy chain comprises a heavy chain variable region and a heavy chain constant region. Each light chain comprises a light chain variable region and a light chain constant region. Complementarity determining regions (CDR) in the variable heavy and/or light chains determine specificity of an antibody towards its antigen.

The antibodies used in the instant methods can be whole antibodies and any antigen binding fragments. An antibody can be a monoclonal antibody, polyclonal antibody, human antibody, humanized antibody, chimeric antibody, and further engineered antibody. Examples of antibodies and fragments thereof include a variable domain fragment (“Fv”, consisting of the VH and VL domains of a single arm of an antibody), Fab fragment (monovalent fragment consisting of the VH, VL, CH1 and CL domains), Fab₂ fragment (bivalent), Fab₃ fragment (trivalent), Fab′ fragment (Fab with hinge region), F(ab′)₂ fragment (bivalent fragment including two Fab fragments linked by a disulfide bridge at the hinge region), Fd fragment (consisting of the VH and CH1 domains), rlgG (reduced IgG or half-IgG), diabodies, triabodies, tetrabodies, minibodies, monovalent antibodies, divalent or multivalent antibodies comprising a fragment of more than one antibody, single chain variable fragment (ScFv), bis-scFv (bispecific), and derivatives of antibodies such as disulfide stabilized Fv fragments, CDR-comprising peptides, as well as epitope-binding fragments of any of the above.

Further, an inhibitor can be a small molecule compound that can inhibit the activity of PTPRZ1 or PTN.

An inhibitor of PTPRZ1 pathway can be provided in the form of a pharmaceutical composition comprising the inhibitor and a pharmaceutically acceptable carrier. Preferred embodiments of the invention provide a pharmaceutical composition comprising an antibody against PTPRZ1 or an antibody fragment thereof, an antibody against PTN or an antigen binding fragment thereof, or an RNAi that inhibits the expression of PTPRZ1 or PTN, such as an shRNA comprising SEQ ID NO: 5 or 6.

In certain embodiments, an inhibitor of the PTPRZ1 or PTN, such as an antibody or antigen binding fragment of the antibody, is conjugated to a chemotherapeutic compound. Such conjugation delivers the chemotherapeutic compound to the PTPRZ1 expressing cancer cell and while the inhibitor inhibits the activity and/or the expression of PTPRZ1 or PTN protein, the chemotherapeutic agent further induces the death of the PTPRZ1 expressing cell.

Chemotherapeutic agents suitable for inducing killing of a cancer cell, particularly, a cancer cell of the nervous system. Non-limiting examples of chemotherapeutic agents include temozolomide, carmustine, and lomustine. Additional such chemotherapeutic drugs are known to a person of ordinary skill in the art and such embodiments are within the purview of the invention.

An inhibitor of the PTPRZ1 pathway can be administered via any suitable route, such as oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In preferred embodiments, an inhibitor of the PTPRZ1 pathway is administered to a subject in a manner that specifically delivers the inhibitor in and around the cancer cells or within the cancer cells. This can be achieved by targeted injection of the inhibitor to the tumor site.

This can be also achieved by packaging the inhibitor in a carrier specifically targeted to the tumor and/or the cancer cells. Such carrier can be liposomes containing binding agents that specifically bind to biomolecules present on the target cancer cells and, thus, specifically deliver the inhibitor of the PTPRZ1 pathway into the target cancer cells.

Further embodiments of the invention provide a method of culturing cancer cells, particularly, primary cancer cells from a cancer, such as, a cancer of the nervous system such as glioblastoma, obtained from a subject, in a brain organoid.

An organoid is a miniaturized and simplified version of an organ produced in vitro in three dimensions that shows realistic micro-anatomy. A brain organoid can be produced from stem cells under conditions that facilitate production of brain cells from the stem cells. Certain details of producing organoids are described by Takebe et al. (2019); Science, Vol. 364, Issue 6444, pp. 956-959. Certain details of producing brain organoids are described by Koo et al. (2019), Mol Cells.; 42(9): 617-627. The Takebe et al. and Koo et al. references are incorporated herein by reference in their entireties.

Accordingly, certain embodiments of the invention provide methods of culturing a PTPRZ1 expressing cancer cell in a brain organoid. The methods comprise:
a) providing a brain organoid;
b) isolating, from a sample of cancer cells obtained from a subject, the cancer cell that express PTPRZ1,
c) transplanting the PTPRZ1 expressing cancer cell into the brain organoid, and
d) culturing the transplanted PTPRZ1 expressing cancer cell.

The brain organoid can be selected from neurospheres, neural aggregates, neural rossettes, cortical spheroids, cortical organoid, cerebral organoid, or whole-brain organoids. The brain organoid is preferably a cortical organoid. Methods of producing brain organoids are well-known in the art and can be used to produce a brain organoid suitable for culturing the PTPRZ1 expressing cells.

In preferred embodiments, the brain organoid is generated from human embryonic stem cells (ESCs), human induced pluripotent stem cells (iPSCs), or a cell line comprising ESCs or iPSCs. For example, a brain organoid can be generated by culturing human ESCs or human iPSCs in a medium comprising a Rho kinase inhibitor, a Wnt signal inhibitor and a TGFβ signal inhibitor on a low adhesion substrate to generate aggregates; and culturing the aggregates in a medium comprising a Wnt signal inhibitor and a TGFβ signal inhibitor and lacking a Rho kinase inhibitor, on a low adhesion substrate to generate the brain organoid. In certain such methods, the Wnt signal inhibitor comprises IWR-1, the TGFβ signal inhibitor comprises SB431542, and the Rho kinase inhibitor comprises Y-27632. Moreover, the brain organoid expresses or is genetically modified to express one or both telencephalon markers, Foxg1 and Six 3.

The step of isolating the PTPRZ1 expressing cells typically comprises disrupting a cancerous mass obtained from a subject, labeling the PTPRZ1 expressing cells with a labeled marker, such as fluorescent labeled PTPRZ1 antibody, and isolating the cells labeled with the marker. Such isolation can be performed using flow cytometry. Methods of isolating a receptor expressing cells using flow cytometry are well-known in the art and can be used to isolate the PTPRZ1 expressing cells.

The isolated PTPRZ1 expressing cells are then transplanted in the brain organoid and the organoid is cultured under appropriate conditions to allow the division of the PTPRZ1 expressing cells within the organoid.

In certain embodiments, after PTPRZ1 expressing cells are isolated but before the cells are transplanted into a brain organoid, the PTPRZ1 expressing cancer cells are transfected with a vector expressing a label. In an exemplary embodiment, the PTPRZ1 expressing cells are transfected with an adenovirus expressing green fluorescence protein.

In preferred embodiments, the PTPRZ1 expressing cancer cells are PTPRZ1 expressing oRG-like cells.

The isolated PTPRZ1 expressing cancer cells, particularly, the PTPRZ1 expressing oRG-like cells, can be used to identify agents that inhibit the PTPRZ1 pathway and, consequently, can also inhibit the tumor invasion. Such agents can be used as therapeutics to prevent the progress and invasion of a cancer of a nervous system.

Accordingly, certain embodiments provide a method for identifying a candidate agent as an inhibitor of invasion by a cancer cell of the nervous system where the cancer cell expresses PTPRZ1. Such method comprises:
a) culturing a first PTPRZ1 expressing cancer cell in the presence of the candidate agent and a second PTPRZ1 expressing cancer cell in the absence of the candidate agent;
b) observing the growth of the first and the second PTPRZ1 expressing cancer cells; and
c) identifying the candidate agent as an inhibitor of PTPRZ1 mediated invasion of the cancer if the first PTPRZ1 expressing cancer cell grows and/or multiplies slower than the second PTPRZ1 expressing cancer cell.

In certain embodiments, the PTPRZ1 expressing cell cultured in a brain organoid in the presence or the absence of a candidate agent. In certain such methods, the MST of the PTPRZ1 expressing cell is observed in the presence and the absence of the candidate agent, and identifying the candidate agent as an inhibitor of PTPRZ1 mediated invasion of the cancer if the PTPRZ1 expressing cancer cell does not grow and/or multiply to the same extent in the presence of the candidate agentas it does in the absence of the candidate agent. A candidate agentis identified as an inhibitor of PTPRZ1 mediated invasion of the cancer if the PTPRZ1 expressing cancer cell does exhibit MST to the same extent in the presence of the candidate agent as it does in the absence of the candidate agent.

In preferred embodiments, a plurality of candidate agents is screened simultaneously to identify one or more candidate agents that can inhibit PTPRZ1 mediated invasion of the cancer. A candidate agent can be an inhibitor of the activity or the expression of PTPRZ1 or PTN. The inhibitors of activity or expression of PTPRZ1 or PTN discussed above, including, the oligonucleotides, antibodies, and small molecules can be used in the screening methods disclosed herein to identify an inhibitor of PTPRZ1 mediated invasion of a cancer.

In preferred embodiments, the methods of identifying a candidate agent as an inhibitor of PTPRZ1 mediated progress or growth of a cancer comprise:
a) providing a first PTPRZ1 expressing cancer cell and a second PTPRZ1 expressing cancer cell;
b) transfecting the first and the second PTPRZ1 expressing cancer cells with a polynucleotide encoding a marker;
c) transplanting the first PTPRZ1 expressing cancer cell into a first brain organoid, and the second PTPRZ1 expressing cancer cell into a second brain organoid;
d) culturing the first PTPRZ1 expressing cancer cell in the absence of the candidate agent and culturing the second PTPRZ1 expressing cancer cell in the presence of the candidate agent;
e) observing the growth of the first and the second PTPRZ1 expressing cancer cells; and
f) identifying the candidate agent as an inhibitor of PTPRZ1 mediated invasion of the cancer if the first PTPRZ1 expressing cancer cell grows and/or multiplies slower than the second PTPRZ1 expressing cancer cell.

In preferred embodiment, the cancer cells expressing PTPRZ1 is an oRG-like glioblastoma cells.

The labeled marker can be a marker suitable for microscopic visualization, such as by fluorescent microscopy. Non-limiting examples of such markers include green fluorescent protein, blue fluorescent protein, cyan-fluorescent protein, enhanced GFP with red-shifted excitation, enhanced yellow-fluorescent protein, or photoactivatable GFP. Additional examples of markers suitable for microscopic visualization are known in the art and such embodiments are within the purview of the invention.

The nucleotide encoding the marker can be in the form of a double stranded linear or circular DNA vector or a viral vector. The viral vector can be an adenoviral vector, Simian Viruses 40 (SV40), polyomavirus, herpesvirus and papovirus. Additional examples of DNA vectors suitable for expression of a marker in a cell are known in the art and such embodiments are within the purview of the invention.

### EXEMPLARY NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

1. A method for culturing primary cancer cells obtained from a subject, the method comprising:
implanting the primary cancer cells into a brain organoid generated from an embryonic stem cell (ESC) or an induced pluripotent stem cells (iPSC), wherein the ESC or iPSC is not genetically modified to render it oncogenic;
culturing the brain organoid comprising the implanted primary cancer cells for a period of at least one week,
wherein the implanted primary cancer cells remain viable for at least one week.

2. The method of aspect 1, wherein the primary cancer cell is present in a composition comprising a population of cells isolated from a tumor tissue obtained from the subject and wherein the implanting comprises implanting the population of cells.

3. The method of aspect 1 or 2, wherein the method comprises resecting a tumor and dissociating the tumor to provide dissociated primary cancer cells.

4. The method of any one of aspects 1-3, wherein the primary cancer cell is obtained from a tumor, wherein the tumor comprises brain tumor, liver tumor, lung tumor, breast tumor, bone tumor, kidney tumor, prostate tumor, ovary tumor, or colon tumor.

5. The method of any one of aspects 1-3, wherein the primary cancer cell is isolated from a cancer of a nervous system.

6. The method of aspect 5, wherein the cancer of the nervous system is brain stem glioma, pineal astrocytic tumor, pilocytic astrocytoma, diffuse astrocytoma, anaplastic astrocytoma, glioblastoma, oligodendroglial tumor, mixed glioma, ependymal tumor, medulloblastoma, pineal parenchymal tumor, meningeal tumor, germ cell tumor, or craniopharyngioma.

7. The method of any one of aspects 1-6, comprising determining growth of cancer cells in the organoid at least one week post-implantation.

8. The method of any one of aspects 1-7, comprising determining the invasiveness of cancer cells in the organoid at least one week post-implantation.

9. The method of aspect 7 or 8, wherein the determining comprises assessing cells expressing a marker specific for the cancer cells.

10. The method of any one of aspects 1-9, wherein the brain organoid is generated from human embryonic stem cells (ESCs).

11. The method of any one of aspects 1-9, wherein the brain organoid is generated from human induced pluripotent stem cells (iPSCs).

12. The method of any one of aspects 1-9, wherein the brain organoid is generated from a cell line comprising human induced pluripotent stem cells (iPSCs).

13. The method of any one of aspects 1-9, wherein the human ESCs or the human iPSCs are not genetically modified.

14. The method of any one of aspects 1-13, wherein the brain organoid is a cortical organoid.

15. The method of aspect 14, wherein the cortical organoid is generated by a method comprising:
culturing human ESCs or human iPSCs in a medium comprising a Rho kinase inhibitor, a Wnt signal inhibitor and a TGFβ signal inhibitor on a low adhesion substrate to generate aggregates; and
culturing the aggregates in a medium comprising a Wnt signal inhibitor and a TGFβ signal inhibitor and lacking a Rho kinase inhibitor, on a low adhesion substrate to generate the cortical organoid.

16. The method of any one of aspects 1-15, wherein the brain organoid expresses one or more of the telencephalon markers Foxg1 and Six 3.

17. The method of aspect 15 or 16, wherein the Wnt signal inhibitor comprises IWR-1.

18. The method of any one of aspects 15-17, wherein the TGFβ signal inhibitor comprises SB431542.

19. The method of any one of aspects 15-18, wherein the Rho kinase inhibitor comprises Y-27632.

20. The method of any one of aspects 1-19, wherein the primary cancer cell expresses protein tyrosine phosphatase receptor type Z1 (PTPRZ1).

21. The method of any one aspects 1-20, further comprising screening a candidate agent for activity in reducing cancer cell growth and/or invasion, the method comprising:
culturing the primary cancer cells in the brain organoid in the presence of the candidate agent and culturing the primary cancer cells in the brain organoid
wherein reduced cancer cell growth of and/or invasion as compared to the cancer cell growth and/or invasion, respectively, in absence of the candidate agent identifies the candidate agent as having activity in reducing cancer cell growth and/or invasion.

22. The method of aspect 21, wherein the candidate agent is a small molecule, oligonucleotide, peptide, or protein.

23. A brain organoid generated from an ESC or an iPSC, the brain organoid comprising implanted therein primary cancer cells derived from a cancer of a nervous system, and wherein the ESC or iPSC is not genetically modified to render it oncogenic.

24. The brain organoid of aspect 23, wherein the ESC or iPSC is a human ESC or human iPSC.

25. The brain organoid of any of aspects 23 or 24, wherein the primary cancer cell is isolated from a brain stem glioma, pineal astrocytic tumor, pilocytic astrocytoma, diffuse astrocytoma, anaplastic astrocytoma, glioblastoma, oligodendroglial tumor, mixed glioma, ependymal tumor, medulloblastoma, pineal parenchymal tumor, meningeal tumor, germ cell tumor, or craniopharyngioma.

26. The brain organoid of any one of aspects 23 to 25, the brain organoid is a cortical organoid.

27. A method for inhibiting the invasion of a cancer of the nervous system, the method comprising administering to a subject suffering from the cancer of the nervous system an inhibitor of protein tyrosine phosphatase receptor type Z1 (PTPRZ1) pathway.

28. The method of aspect 27, wherein the cancer is in the brain, spinal cord, or a neuroendocrine gland.

29. The method of aspect 27 or aspect 28, wherein the cancer is brain stem glioma, pineal astrocytic tumor, pilocytic astrocytoma, diffuse astrocytoma, anaplastic astrocytoma, glioblastoma, oligodendroglial tumor, mixed glioma, ependymal tumor, medulloblastoma, pineal parenchymal tumor, meningeal tumor, germ cell tumor, or craniopharyngioma.

30. The method of any one of aspects 27-29, wherein the inhibitor of the PTPRZ1 pathway is an inhibitor of the expression or activity of PTPRZ1 or an inhibitor of the expression or activity of pleiotrophin (PTN).

31. The method of aspect 30, wherein the inhibitor of PTPRZ1 expression is an oligonucleotide that specifically inhibits the transcription of PTPRZ1 mRNA or translation of PTPRZ1 protein.

32. The method of aspect 30, wherein the oligonucleotide that specifically inhibits the transcription of PTPRZ1 mRNA is an interfering RNA (RNAi).

33. The method of aspect 32, wherein the RNAi is small interfering RNA (siRNA), double-stranded RNA (dsRNA), single-stranded RNA (ssRNA), micro-RNA (miRNA), or short hairpin RNA (shRNA) that specifically inhibits the transcription of PTPRZ1 mRNA.

34. The method of aspect 30, wherein the inhibitor of PTN expression is an oligonucleotide that specifically inhibits the transcription of PTN mRNA or translation of PTN protein.

35. The method of aspect 34, wherein the oligonucleotide that specifically inhibits the transcription of PTN mRNA is an interfering RNA (RNAi).

36. The method of aspect 35, wherein the RNAi is small interfering RNA (siRNA), double-stranded RNA (dsRNA), single-stranded RNA (ssRNA), micro-RNA (miRNA), or short hairpin RNA (shRNA) that specifically inhibits the transcription of PTN mRNA.

37. The method of aspect 30, wherein the inhibitor of activity of PTPRZ1 is an antibody or an antigen binding fragment of an antibody that specifically binds to PTPRZ1, optionally, conjugated to a chemotherapeutic agent.

38. The method of aspect 37, wherein the antibody is a monoclonal antibody, polyclonal antibody, human antibody, humanized antibody, chimeric antibody, a variable domain fragment, Fab fragment, Fab₂ fragment, Fab₃ fragment, Fab′ fragment, F(ab′)₂ fragment, Fd fragment, rlgG, dibody, tribody, tetrabody, minibody, monovalent antibody, divalent antibody, multivalent antibody, single chain variable fragment (ScFv), or bis-scFv that specifically binds to PTPRZ1 .

39. The method of aspect 30, wherein the inhibitor of activity of PTN is an antibody or an antigen binding fragment of an antibody that specifically binds to PTN, optionally, conjugated to a chemotherapeutic agent.

40. The method of aspect 39, wherein the antibody is a monoclonal antibody, polyclonal antibody, human antibody, humanized antibody, chimeric antibody, a variable domain fragment, Fab fragment, Fab₂ fragment, Fab₃ fragment, Fab′ fragment, F(ab′)₂ fragment, Fd fragment, rlgG, dibody, tribody, tetrabody, minibody, monovalent antibody, divalent antibody, multivalent antibody, single chain variable fragment (ScFv), or bis-scFv that specifically binds to PTN.

41. The method of aspect 30, wherein the inhibitor of PTPRZ1 or PTN expression or activity is a small molecule compound.

42. The method of any one of aspects 27-41, comprising administering the inhibitor of the PTPRZ1 pathway specifically in and around the cancerous tissue of the subject.

43. The method of any one of aspects 27-42, comprising packaging the inhibitor in a carrier that is specifically targeted to the cancer cells.

44. A method for culturing a PTPRZ1 expressing cancer cell, the methods comprising:
a) providing a brain organoid;
b) isolating, from a sample of cancer cells obtained from a subject, the cancer cell that express PTPRZ1,
c) transplanting the PTPRZ1 expressing cancer cell into the brain organoid, and
d) culturing the transplanted PTPRZ1 expressing cancer cell.

45. The method of aspect 44, wherein the brain organoid is a neurosphere, neural aggregate, neural rossette, cortical spheroid, cortical organoid, cerebral organoid, or whole-brain organoid.

46. The method of aspect 44 or aspect 45, wherein the step of isolating the PTPRZ1 expressing cancer cell comprises disrupting a cancerous mass obtained from the subject, labeling the PTPRZ1 expressing cell with a marker, and isolating the cell labeled with the marker.

47. The method of aspect 46, wherein the marker is as fluorescent labeled PTPRZ1 antibody and isolating comprises performing flow cytometry.

48. The method of any one of aspects 44 to 47, further comprising, after isolating the PTPRZ1 expressing cancer cell but before transplanting the PTPRZ1 expressing cancer cell into the brain organoid, transfecting the PTPRZ1 expressing cancer cell with a polynucleotide encoding a label.

49. The method of any one of aspects 44 to 48, wherein the PTPRZ1 expressing cancer cell is a PTPRZ1 expressing outer radial glia-like cell.

50. A method for identifying a candidate agent as an inhibitor of cancer cell invasion, wherein the cancer cell expresses PTPRZ1, the method comprising:
culturing a first PTPRZ1 expressing cancer cell in the presence of the candidate agent and a second PTPRZ1 expressing cancer cell in the absence of the candidate agent;
wherein reduced cancer cell of the first PTPRZ1 expressing cancer cell as compared to the second PTPRZ1 expressing cancer cells identifies the candidate agent as an inhibitor of cancer cell invasion.

51. The method of aspect 50, comprising culturing the first and the second PTPRZ1 expressing cancer cells in a brain organoid.

52. The method of aspect 51, comprising assessing the mitotic somal translocation (MST) of the first and the second PTPRZ1 expressing cancer cell and identifying the candidate agent as an inhibitor of cancer cell invasion if the first PTPRZ1 expressing cancer cell exhibits lower MST than that of the second PTPRZ1 expressing cancer cell.

53. The method of any one of aspects 50-52, comprising transfecting the first and the second PTPRZ1 expressing cancer cells with a polynucleotide encoding a marker and transplanting the first PTPRZ1 expressing cancer cell into a first brain organoid, and the second PTPRZ1 expressing cancer cell into a second brain organoid prior to culturing the first PTPRZ1 expressing cancer cell in the absence of the candidate compound and culturing the second PTPRZ1 expressing cancer cell in the presence of the candidate agent.

54. The method of any one of aspects 50-53, wherein the first and second PTPRZ1 expressing cancer cell is an oRG-like glioblastoma cell.

55. The method of aspect 53 or aspect 54, wherein the marker is green fluorescent protein, blue fluorescent protein, cyan-fluorescent protein, enhanced GFP with red-shifted excitation, enhanced yellow-fluorescent protein, or photoactivatable GFP.

56. The method of any one of aspects 50 to 55, further comprising observing the MST of the first and the second PTPRZ1 expressing cancer cells and identifying the candidate agent as an inhibitor of cancer cell invasion if the first PTPRZ1 expressing cancer cell exhibits lower MST than that of the second PTPRZ1 expressing cancer cell.

57. The method of any one of aspects 53 to 56, wherein the nucleotide encoding the marker comprises a double stranded linear or circular DNA vector.

58. The method of aspect 57, wherein the DNA vector is a viral vector.

59. The method of aspect 58, wherein the viral vector is an adenoviral vector, Simian Virus 40 (SV40) vector, polyomaviral vector, herpesviral vector, or papoviral vector. The following example is offered by way of illustration and not by way of limitation.

### Experimental

### Materials and Methods

### Mouse Transplantation of GBM Cells

All mouse experiments were approved by and performed according to the guidelines. Dissociated GBM cells from fresh surgical resections were concentrated by centrifugation at 800 × g for 3 min. P2 CD1 mice recipients were anesthetized using hypothermia until pedal reflex disappeared and then were placed on a stereotaxic injection platform. Cells were loaded into a beveled glass micropipette (Drummond Scientific) positioned at 30° from vertical. A single injection was placed into the right cortex using the following coordinates (with regards to Lambda): anterior-posterior (A-P) -1.8 mm; medial-lateral (M-L) 2 mm. The depth was 0.7 mm from the surface of the skin. After injection, transplanted animals were placed on a heating pad until warm and active and were returned to their mothers until weaning age (P21). For immunosuppressive therapy protocol, recipient neonatal mice were treated with anti-CD40L (MR-1), anti-LFA-1 (M17/4), and CTLA4-lg (BioXCell) as previously described (Pearl et al., 2011). Anti-CD40L, anti LFA-1 and CTLA4-lg were administered intraperitoneally at a dose of 20 mg/kg on days 0, 2, 4, and 6 after transplantation.

All procedures were carried out under sterile conditions. Mice was anesthetized by intraperitoneal injection of a mixture solution containing ketamine (100 mg/kg) and xylazine (7.5 mg/kg). The scalp was surgically prepared, and ~ 20 ul of 0.25% Bupivacaine was injected intra-cutaneous space of the scalp. A skin incision ~ 10 mm in length was made over the middle frontal to parietal bone. The surface of the skull was exposed, and a small hole was made using a 25-gauge needle 3 mm to the right of the bregma and just in front of the coronal suture. A 26-gauge needle attached to a Hamilton syringe was inserted into the skull hole. The needle was covered with a sleeve that limits the depth of 4 mm injection. Cell suspensions in 3 µl was injected very slowly (~ 1 µl/ minute) by free hand and then the needle was removed. The skull surface was swabbed with hydrogen peroxide before the hole is sealed with bone wax to prevent reflux. The scalp was closed with surgical staples. F1 tumors were continued for 69 days (until mouse morbidity) while F2 tumors were harvested after 16 days due to mouse morbidity.

### Tumor Collection and Dissociation

Fresh tumor tissues were acquired from patients undergoing surgical resection. De-identified samples were provided. Sample use was approved by the Institutional Review Board and experiments conform to the principles set out in the WMA Declaration of Helsinki and the Department of Health and Human Services Belmont Report. All patients provided informed written consent. Tumors were collected in media consisting of Leibovitz’s L-15 medium, 4 mg/mL glucose, 100 u/mL Penicillin, 100 ug/mL Streptomycin. Tumor tissues were mechanically and enzymatically dissociated using a papain-based brain tumor dissociation kit (Worthington). The dissociated, single cells were resuspended in GNS (Neurocult NS-A (Stem Cell Tech.), 2 mM L-Glutamine, 100 U/mL Penicillin, 100 ug/mL Streptomycin, N2/B27 supplement (Invitrogen), sodium pyruvate).

### Single-Cell RNA Sequencing

Single-cell RNA sequencing was performed either using the Fluidigm C1 system or the 10X Chromium v2 system. For the Fluidigm captures, single cell capture, cDNA synthesis and preamplification was performed as described before(Nowakowski et al., 2017; Pollen et al., 2015; Pollen et al., 2014) using Fluidigm C1 auto-prep system following manufacturer’s protocol. Library preparation was performed using the Illumina Nextera XT library preparation kit. Library concentration quantification was performed using Bioanalyzer (Agilent). Paired-end 100 bp sequencing was performed on the Illumina HiSeq 2500.

10X Chromium v2 captures were performed on dissociated tumor samples with a target capture of 2,000 cells per lane. For each tumor, 2 lanes were used for capture. Library prep was performed using manufacturer instructions (10X) and sequencing was performed on the Illumina HiSeq 2500. A larger number of cells was obtained with the 10X Chromium system, but the overall tumor composition between the two platforms was similar suggesting no additional bias was introduced.

### Processing of Adult Human Brain Tissue for Single Nucleus RNA Sequencing

De-identified snap-frozen post-mortem tissue samples from individuals with no medical history of neurological disease or pathological changes of brain tissue were obtained. Cortical samples encompassing the entire span of the cortex were sectioned on a cryostat to collect 100 um sections for total RNA isolation and nuclei isolation. In case of presence of subcortical white matter, white matter was dissected out before collecting the sections to ensure obtaining all layers of the cortical grey matter. Total RNA from ~ 10 mg of collected tissue was isolated and used to perform RNA integrity analysis on the Agilent 2100 Bioanalyzer using RNA Pico Chip assay. Only samples with RNA integrity number (RIN) >6.5 were used to perform nuclei isolation and single-nucleus RNA sequencing (snRNA-seq).

### Nuclei Isolation and SnRNA-Seq on the 10X Genomics Platform

Single-nuclei sequencing was only used for the reference adult dataset, as published in Velmeshev et al. 40 mg of sectioned brain tissue was homogenized in 5 mL of RNAase-free lysis buffer (0.32 M sucrose, 5 mM CaCl2, 3 mM MgAc2, 0.1 mM EDTA, 10 mM Tris-HCl, 1 mM DTT, 0.1% Triton X-100 in DEPC-treated water) using glass dounce homogenizer (Thomas Scientific, Cat # 3431D76) on ice (Matevossian and Akbarian, 2008). The homogenate was loaded into a 30 mL thick polycarbonate ultracentrifuge tube (Beckman Coulter, Cat # 355631). 9 mL of sucrose solution (1.8 M sucrose, 3 mM MgAc2, 1 mM DTT, 10 mM Tris-HClin DEPC-treated water) was added to the bottom of the tube with the homogenate and centrifuged at 107,000 g for 2.5 hours at 4° C. Supernatant was aspirated, and nuclei pellet was incubated in 250 µL of DEPC-treated water-based PBS for 20 min on ice before resuspending the pellet. Nuclei were counted using a hemocytometer and diluted to 2,000 nuclei/µL before performing single-nuclei capture on the 10X Genomics Single-Cell 3′ system. Target capture of 3,000 nuclei per sample was used; the 10X capture and library preparation protocol was used without modification. Matched control and ASD samples were loaded on the same 10X chip to minimize potential batch effects. Single-nuclei libraries from individual samples were pulled and sequenced on the Illumina HiSeq 2500 machine, 1 sample per lane (average depth 80,000 reads/nucleus).

### SnRNA-Seq Data Processing With 10X Genomics CellRanger Software and Data Filtering

For library demultiplexing, fastq file generation and read alignment and UMI quantification, CellRanger software v 1.3.1 was used. CellRanger was used with default parameters, except for using pre-mRNA reference file (ENSEMBL GRCh38) to insure capturing intronic reads originating from pre-mRNA transcripts abundant in the nuclear fraction. Individual expression matrices containing numbers of Unique molecular identifiers (UMIs) per nucleus per gene were filtered to retain nuclei with at least 500 genes expressed and less than 5% of total UMIs originating from mitochondrial and ribosomal RNAs. Genes expressed in less than three nuclei were filtered out. Mitochondrial RNA genes were filtered out as well to exclude transcripts coming from outside the nucleus to avoid biases introduced by nuclei isolation and ultracentrifugation. Individual matrices were combined, UMIs were normalized to the total UMIs per nucleus and log transformed.

### Dimensionality Reduction, Clustering and T-SNE Visualization of SnRNA-Seq Data

Filtered log transformed UMI matrix was used to perform truncated singular value decomposition (SVD) with k=50. Scree plot was generated to select the number of significant principle components (PCs) by localizing the last PC before the explained variance reaches plateau. The significant PCs were used to calculate Jaccard distance-weighted nearest neighbor distances; number of nearest neighbors was assigned to root square of number of nuclei. Resulting graph with Jaccard-weighted edges was used to perform Louvain clustering. To visualize nuclei transcriptomic profiles in two-dimensional space, t-distributed stochastic neighbor embedding (t-SNE) was performed with the selected PCs and combined with cluster annotations.

### Cell Type Annotation and Quantification of Regional and Individual Contribution to Cell Types for SnRNA-Seq Dataset

Cell types were annotated based on expression of known marker genes visualized on the t-SNE plot and by performing unbiased gene marker analysis. For the latter, MAST (Finak et al., 2015) was used to perform differential gene expression analysis by comparing nuclei in each cluster to the rest of nuclei profiles. Genes with FDR < 0.05 and log fold change of 1 or more were selected as cell type markers. To get insight into regional enrichment of cell types, numbers of nuclei in each cluster were normalized to the total number of nuclei captured from each region.

### Alignment

For C1 captured cells: Trim Galore 3.7 was used to trim 20 bp of adaptor sequencing and paired-end alignments were performed using HISAT2 to the human reference genome GRCh38. Counts for each cell were performed using subread-1.5.0 function featureCounts. After counts were obtained, normalization to counts per million was performed. For 10X captured cells: The cells were demultiplexed and aligned using the cellranger 2.0 software.

Quality control was performed to further ensure that only high-quality single cell data was processed further, and cells with fewer than 1000 genes/cell were removed, as were cells with greater than 10% of their individual transcriptome represented in either mitochondrial or ribosomal transcripts. Only genes expressed in at least 30 cells were carried forward in the analysis.

### Clustering Analysis and Correlations to Cell Types

Batch correction was performed to account for the C1 platform and the 10X platform, as well as any variation introduced by day-to-day variability. An approach adapted from Peng et al. (2019) was used. For each batch (as defined by day of capture for 10X samples, all C1 samples were considered a single batch), the counts were normalized to the largest number and then multiplied by the median number of counts per cell for the entire batch. During scaling (using the Seurat v2 ScaleData function, batch was regressed out in the space of variable genes only). Clustering was performed as previously described by Shekhar et al. (2016). Briefly, principal component analysis was performed on the full matrix of expressed genes and cells passing quality control. Significant principal components were used to score each cell and nearest neighbor analysis (RANN package) was performed with k = 10. Jaccard weighted distances were used as the input from this nearest neighbor analysis for Louvain clustering (igraph package) and tSNEs were generated in PCA space.

Correlations to cell type were generated using marker genes of known cell types from developing human single-cell RNA sequencing Nowakowski et al. (2017) and adult single nuclei Velmeshev et al. (unpublished). For cluster level analyses, marker genes were assigned a “gene score” based upon the fold change multiplied by the enrichment of the marker in the relevant cluster compared to all others. Correlations were performed between marker sets based upon the intersection of marker genes between the two datasets being compared. Correlations between known clusters and GBM clusters > 0.4 were designated by the best correlation. Correlations between 0.2 and 0.4 were used as a guide, but cluster markers were carefully cross referenced with literature to ensure accurate cell type designations. For correlations < 0.2, literature searches on enriched genes were used to make cell type identifications.

For cell level analyses, each set of cluster markers for radial glia subtypes was defined as a network and was used as an input for module eigengene calculation by using the loading of the first principal component in the space of this gene set. In heatmaps, positive and negative scores were used, whereas in histogram graphs only positive scores were used. The radial glia gene co-expression network was generated by calculating the correlation outer radial glia marker genes as described by Pollen et al. (2015) in the space of a restricted matrix with only radial glia cells and glioblastoma (GBM) radial glia-like cells. Edges are defined by the strength of the correlation (Pearson’s R), nodes were colored by number of interactions as designated in the legend, and only genes with correlations of R > 0.30 were utilized in the network. Network representation was generated used Cytoscape 3.0.

### CNV Analysis of Tumor Versus Normal Cells

The presence/absence of somatic copy-number alterations (CNVs) was assessed with CONICSmat. Briefly, raw counts in cells of each patient were scaled to log(CPM/100+1) and centered by the average expression in each cell. Next, the average expression of all genes on each chromosome was calculated in every cell, whereas only genes robustly (log(CPM/100+1))>1) expressed in more than 10 cells were considered. Subsequently, a two-component Gaussian mixture model was fitted on the average expression values across all cells for each chromosome. Chromosomes were investigated with a significant deviation of the log-likelihood of the model compared to a one-component model (likelihood ratio test < 0.001) and a difference in Bayesian Inference Criterion (BIC) >300. These chromosomes were considered to have somatic CNVs. For each of these regions we used a cutoff on the posterior probability (pp > 0.8) to infer the presence/absence of the respective CNV in a cell. Cell with CNV alterations were classified as tumor cells, whereas cells with statistical probabilities suggesting no CNV alterations were classified as normal cells. Cells that could not be clearly assigned to a genotype (e.g. 0.2 < pp < 0.8) remained unclassified. In addition, to validate the approach, several alternative strategies were used to verify the results. Scanning a 100 bp window as has been previously reported and then using the above strategy verified the calls matched. As in Muller et al. (2018), an empirical FDR was calculated for each sample. Moreover, the approach was validated using expression-based validation of gene signatures as described by Yuan et al. (2018). Additionally, the calls from CONICSmat were validated by exploring transcriptomic annotation as described by Petti et al. (2018). For three of the samples, the calls were also compared to those from exome sequencing and matched very well. Once CNV status was used to identify tumor and normal cells, these designations were intersected with cell type identity. CNV hierarchies were created using parsimonious explanations of the sequence of CNV events.

### Viral Infection and Time-Lapse Imaging of Dissociated Tumor Cells

Dissociated tumor cells were seeded onto matrigel coated glass bottom wells. 24 hours after plating cells were infected with a CMV-adeno-GFP virus (Vector Biolabs) to allow for visualization of cell morphology and behavior. Cell were cultured at 37° C., 5% CO₂, and 8% O₂, and monitored for GFP and expression (~ 24 to 48 h). Cultured cells were then transferred to an inverted Leica TCS SP5 with an on-stage incubator (while streaming 5% CO₂, 5% O₂, balance N₂ into the chamber), and imaged in both the GFP and transmitted light channels using a × 10 objective at 15-20 min intervals for up to 4. Movies were analyzed with Imaris.

### Fluorescence Activated Cell Sorting Purification of PTPRZ1 Positive Cells

Previously adherently cultured or fresh tumor sample GBM cells were dissociated into a single-cell suspension with Accutase (Innovative Cell Technologies) for 5 minutes or papain (Worthington) by digestion for one hour. In the context of plated cells, cell viability was higher with Accutase compared to papain, while with tissue it was higher with papain. After digestion, cell suspensions were triturated and placed on top of 4 ml 22% Percoll. Tubes with Percoll and cell suspension were spun at 500 g for 10 minutes without break. The supernatant was discarded, and the cell pellet resuspended in HBSS with BSA and glucose. Cells were then incubated with PTPRZ1 antibody (Atlas Antibodies HPA015103), washed with HBSS with BSA and glucose and then stained with AlexaFluor488 (Thermo Fisher Scientific). Cells were sorted using a Becton Dickinson FACSAria using 13 psi pressure and 100 µm nozzle aperture. All FACS gates were set using unlabeled cells. Additionally, secondary only controls were used to analyze levels of background staining. Data was analyzed post hoc for enrichment percentages with FlowJo software. Enriched cells were infected with CMV-adeno-GFP virus (Vector Biolabs) for visualization of cell morphology and behavior and either replated for live imaging or directly taken for single-cell sequencing and mouse or organoid transplantations.

### Sequencing of PTPRZ1 Knockdown

Primary cells (GW 17, n = 2) and primary tumor samples (n = 2) were dissociated and infected with lentivirus either with a hairpin against PTPRZ1 mRNA or a control hairpin during plating. Cells were maintained in their normal media conditions for 1 week, after which cells were removed from the plate with trypsinization, dissociated into a single-cell suspension, and captured with 10X v2 single-cell as previously described.

### Tyramide Signal Amplification for Immunohistochemistry of FFPE Tumor Samples

FFPE slides were deparaffinized through successive immersions in xylene and ethanol. Slides were immersed in xylene twice, 10 minutes each time. Slides were then immersed in 100% ethanol twice, 2 minutes each time, 95% ethanol once for 2 minutes, 70% ethanol once for 2 minutes, 50% ethanol once for 2 minutes. Slides were then rinsed in MilliQ water and immersed in PBS once for 10 minutes.

Invitrogen’s AlexaFluor 488 Tyramide SuperBoost kit (B40943) and AlexaFluor 594 Tyramide SuperBoost kit (B40942) were used according to the manufacturer’s protocol. Samples were blocked in the kit’s blocking buffer for 1 hr. Samples were then incubated with primary antibodies diluted in the same blocking buffer overnight at 4° C. Slides were rinsed in PBS. Samples were incubated in kit’s secondary antibodies for 1 h at room temperature. Slides were rinsed in PBS. Samples were then incubated with the tyramide working solution prepared according to manufacturer recommendations for 10 minutes at room temperature. Stop solution prepared according to manufacturer’s recommendations was added to samples in order to quench tyramide reaction at the end of the 10-minute incubation period. Slides were rinsed in PBS. Samples were incubated in DAPI diluted 1:1000 in kit blocking buffer for 10 minutes. Slides were rinsed in PBS, then mounted in Southern Biotech Fluoromount (0100-01).

Primary antibodies used were Santa Cruz rabbit HOPX antibody (sc-30216) at 1:50, Santa Cruz mouse Ptprz1 antibody (sc-33664) at 1:200, Santa Cruz mouse Sox2 antibody (sc-365823) at 1:200, Cell Signaling Technologies rabbit PDGFR alpha antibody (3174S) at 1:200, Abcam rabbit Glast antibody (ab416) at 1:200, Millipore rabbit Olig2 antibody (AB9610) at 1:200, Abcam mouse Satb2 (ab51502) at 1:100, Agilent mouse CD31 (GA610) at 1:200, Dako mouse KI67 (M724001) at 1:200.

### Cortical Slice Culture, Viral Infection, and Time-Lapse Imaging

As described by Hansen et al. (2010), human fetal brain tissue was collected and transported in artificial CSF (ACSF; 125 mm NaCl, 2.5 mm KCI, 1 mm MgCl₂, 2 mm CaCl₂, 1.25 mm NaH₂PO₄, 25 mm NaHCO₃, 25 mm d-(+)-glucose, bubbled with 95%O₂/5%CO₂). Brain tissue was embedded in 3% low melting point agarose in ACSF and 250-300 µm coronal vibratome sections were generated. Sections were transferred on Millicell-CM slice culture inserts (Millipore) and cultured in cortical slice culture medium (66% BME, 25% Hanks, 5% FBS, 1% N-2, 1% penicillin, streptomycin and glutamine, all Invitrogen, and 0.66% d-(+)-glucose, Sigma-Aldrich). High titer gfp labeled lentivirus was applied directly to slices that were then cultured at 37° C., 5% CO₂, 8% O₂ for ~ 96 h. For time-lapse imaging, cultures were then transferred to an inverted Leica TCS SP5 with an on-stage incubator streaming 5% CO₂, 5% O₂, balance N2 into the chamber. Slices were imaged for GFP using a 10x objective at 15-30 min intervals for up to 3 d with repositioning of the z-stacks every ~12 h. Movies were analyzed with Imaris.

### IPSO and Organoid Culture

Human induced pluripotent stem cells (iPSCs) from the H28126 line were maintained using feeder-free conditions on Matrigel (BD) coated dishes in meters (Stem Cell Technologies) medium. iPSCs were differentiated using a modified Sasai organoid protocol for directed telencephalon differentiation. iPSCs were dissociated using Accutase (Stem Cell Technologies) and aggregated into 96 well v-bottom low adhesion plates (S-bio). Aggregates were cultured in media containing Glasgow-MEM, 20% Knockout Serum Replacer, 0.1 mM NEAA, 1 mM sodium pyruvate, 0.1 mM β-ME, 100 U/mL penicillin/streptomycin and supplemented with Rho Kinase, Wnt and TGFβ inhibitors, 20 uM Y-27632 (Tocris), 3 uM IWR1endo (Cayman), and 5 µM SB431542, respectively (Tocris). Rho Kinase inhibitor was removed after 6 days. Media was changed every other day throughout differentiation. After 18 days, organoids were transferred into 6 well low-adhesion plates in media containing DMEM/F12 with 1x Glutamax, 1x N2, 1x Lipid Concentrate, and 100 U/mL penicillin/streptomycin. After five weeks, organoids are matured in media containing DMEM/F12 with Glutamax, 1 x N2, 1 x Lipid Concentrate, 100 U/mL penicillin/streptomycin, 10x Fetal Bovine Serum (Hyclone), 5 ug/ml Heparin and 0.5% Growth factor-reduced Matrigel. After 10 weeks the concentration of Matrigel is increased to 1% and the media is additionally supplemented with 1x B-27.

### Whole Organoid Immunostaining, Clearing and Imaging

Human brain organoids were fixed at room temperature for 45 minutes in 4% paraformaldehyde. After fixation, they were washed 3 times in PBS and stored at 4° C. For whole-organoid immunostaining and tissue clearing, the organoids were blocked for 24 hours at room temperature in PBS supplemented with 0.2% gelatin (VWR, 24350.262) and 0.5% Triton X-100 (Millipore Sigma, X100) (PBSGT). Samples were then incubated with Goat anti GFP primary antibody (1:100; Abcam, ab6658) for 7 days at 37° C. at 70 rpm in PBSGT + 1 mg/ml Saponin Quillaja sp (Sigma Aldrich, S4521) (PBSGTS). Following primary antibody incubation, samples were washed 6 times in PBSGT over the course of one day at room temperature. Secondary antibody staining was done using the Alexa Fluor 488 Donkey anti goat antibody (ThermoFisher Scientific, A-11055) and the nucleus was stained using Syto17 dye (ThermoFisher Scientific, S7579). Secondary and nuclear staining was performed at for 1 day at 37C at 70 rpm in PBSGTS. Samples were then washed 6 times in PBSGT over the course of one day at room temperature.

Whole organoid clearing was performed using ScaleCUBIC-1 solution as described in Suzaki et al. (2015). Briefly, the solution contained: 25% by weight Urea (Millipore Sigma, U5378), 25% by weight N,N,N′,N′-Tetrakis(2-hydroxypropyl) ethylenediamine (Tokyo Chemical Industry, T0781) and 15% Triton X-100 dissolved in distilled water. Organoids were incubated in ScaleCUBIC-1 solution overnight at room temperature at 90 rpm.

Whole organoid imaging was performed using a custom-made Lattice Light Sheet Microscope (UCSF Biological Imaging Development Center) and the images were deconvoluted using Richardson-Lucy algorithm. Images were then processed using Imaris 9.2 software (Bitplane).

### Statistical Analysis

Quantification of images was performed with Imaris or ImageJ as described in the relevant methods and figure legends. Statistical analysis for cluster markers and differential gene expression was performed using Seurat v2, the exact statistical test is indicated in the relevant legend and method. Remaining statistical comparisons were calculated using Prism v7 (GraphPad) and are indicated in the relevant figure legends.

### Data and Code

RNA sequencing data has been deposited at PRJNA579593 and SRP132816 (snRNA-seq).

### Example 1 - Identifying Glioblastoma Cell Composition

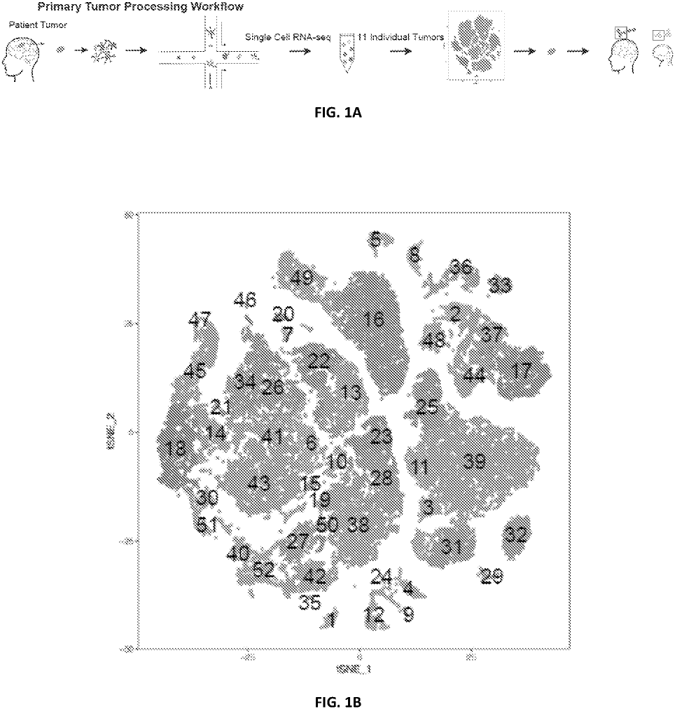

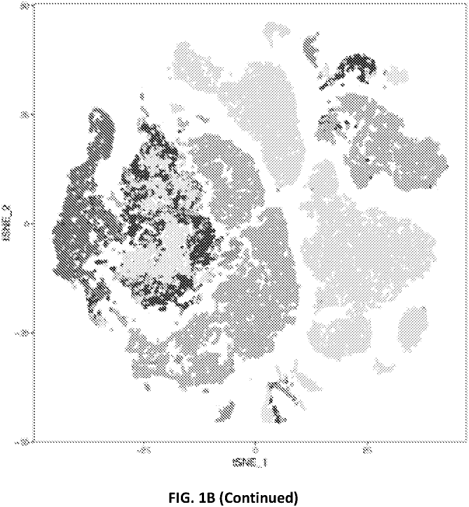

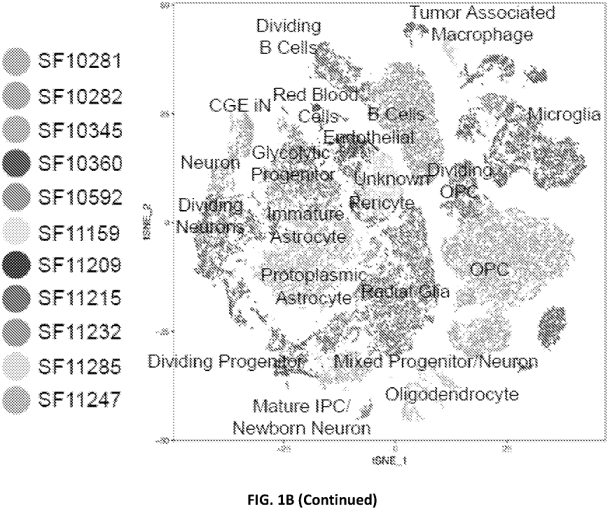

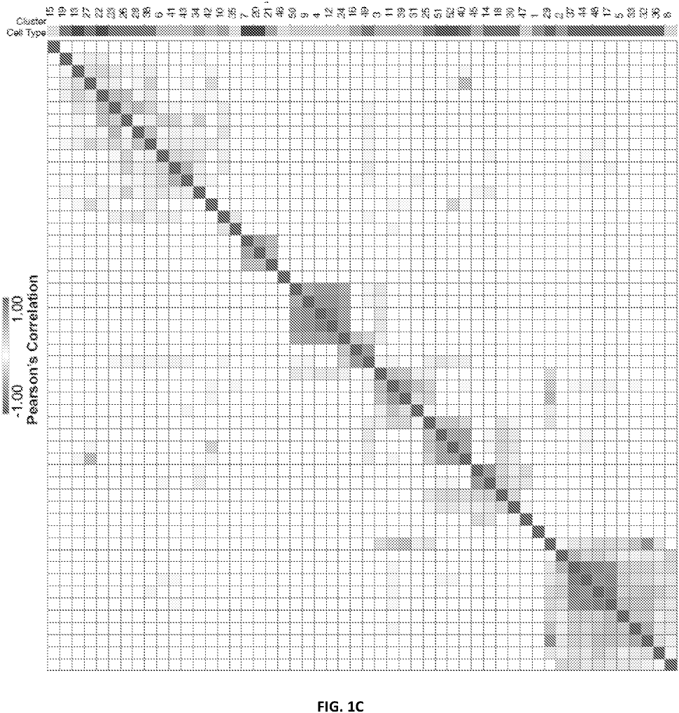

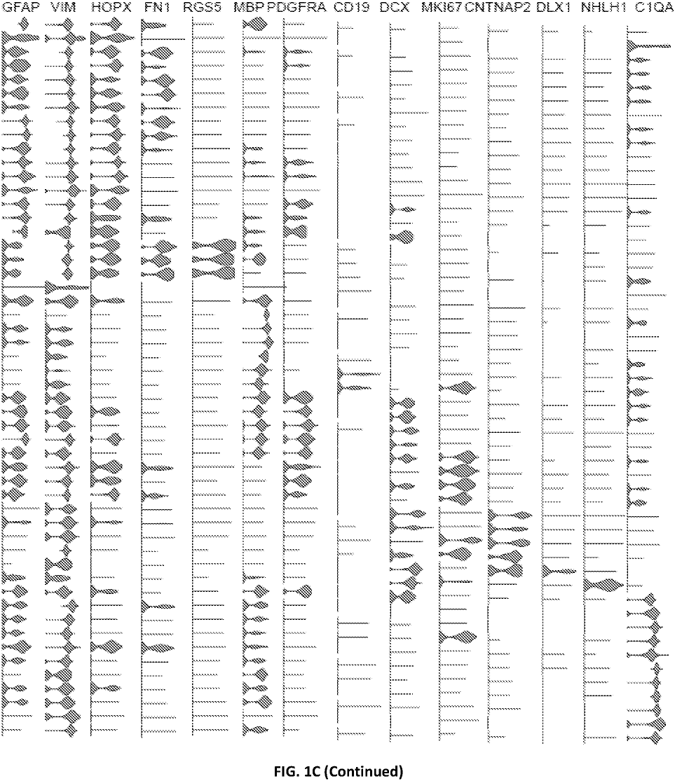

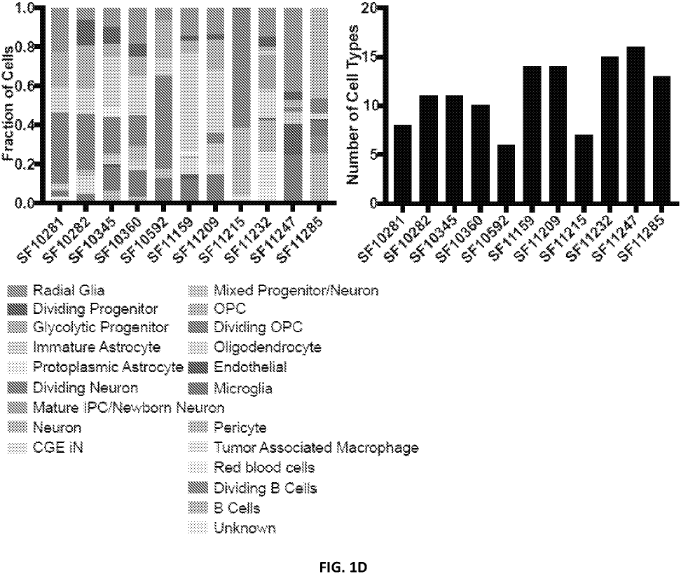

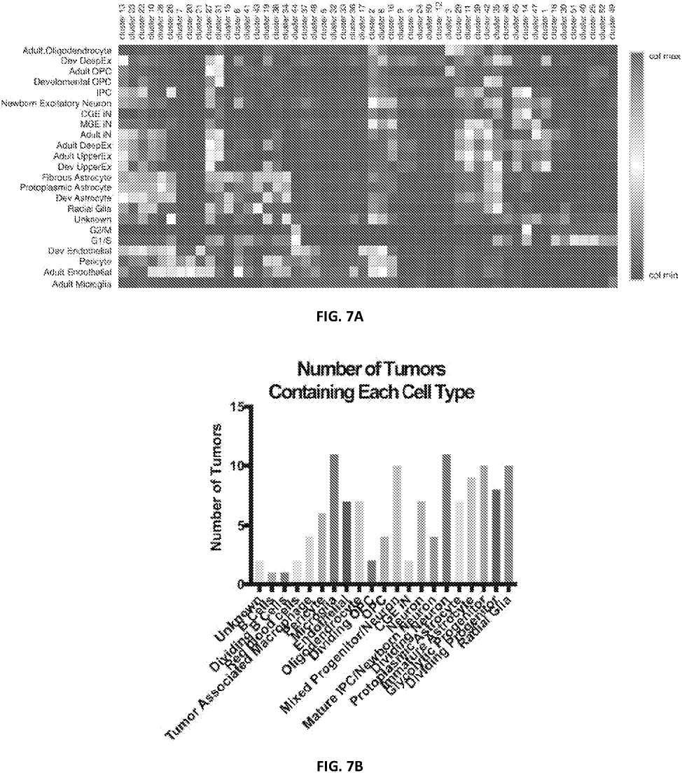

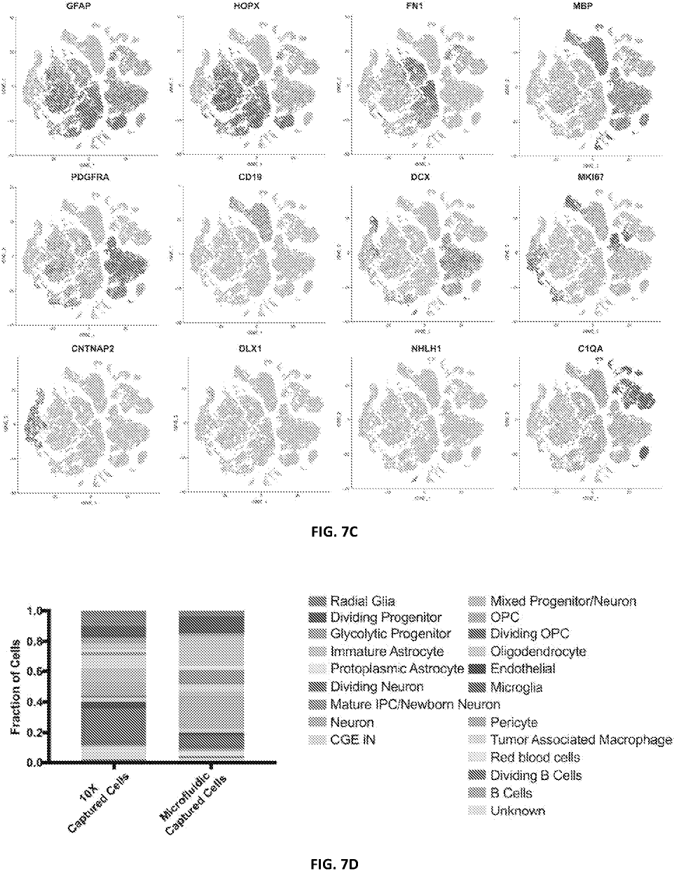

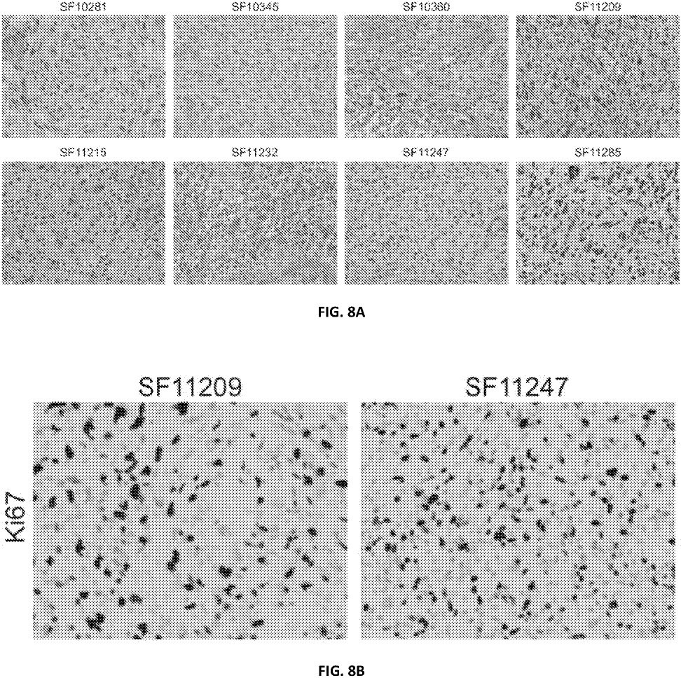

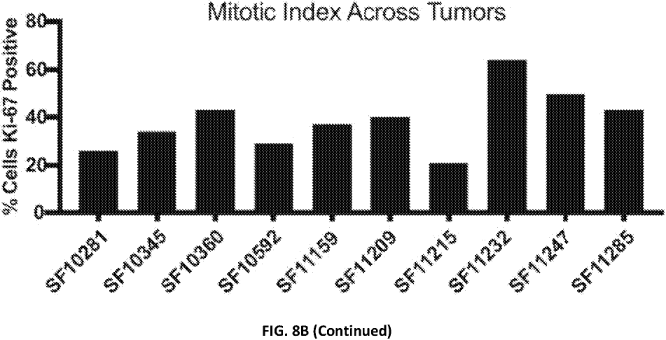

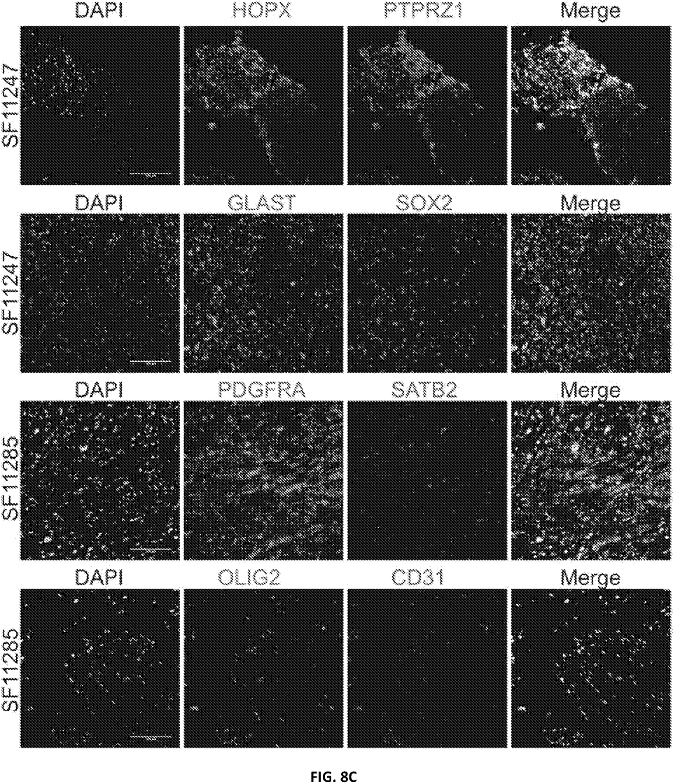

To examine cell composition within glioblastoma, single-cell RNA sequencing was performed of 32,877 cells from 11 tumors dissociated and processed directly after surgical tumor resections (FIG. 1A). The marker genes were compared from clustering analysis to previously described transcriptional signatures of adult cortex (single-nuclei sequencing) by Velmeshev et al. (2019) or developing human brain (single-cell sequencing) by Nowakowski et al. (2017). Closest primary, normal cell type for each glioblastoma cluster was identified. With this analysis, populations were observed that have been previously observed in glioblastoma, including oligodendrocyte precursor cells (OPCs), microglia, tumor associated macrophages, and dividing tumor cells. In addition, populations were identified of neurons (including neurons co-expressing progenitor markers or cell cycle genes), radial glia, and glial cell populations with varying degrees of maturity (FIG. 1B, FIGS. 7A-7D). Each of these progenitor, glial and neuronal populations had strong expression of identifiable marker genes that characterize the cell type, including GFAP, VIM, DCX, MKI67, and DLX1. However, unlike in primary samples, despite clear expression of these markers, instances were observed of co-expression of markers of progenitor identity and markers of post-mitotic neurons (FIG. 1C). Although each of the annotated cell populations is referenced as a ‘type,’ the permanence of cell identity and cell ‘state’ remains unknown in both the context of normal development and in tumorigenesis. Many cell states have the potential to give rise to tumor cells characteristic of the remaining tumor. In the experiments disclosed here, in each tumor contained a distinctive combination of cell type/state heterogeneity, with each tumor on average containing 11 transcriptional cancer cell types (FIG. 1D and FIG. 7B), and this observation was validated in the re-analysis of other recent single-cell RNA-sequencing datasets from primary glioblastoma tumors (FIG. 7D). Using hematoxylin and eosin staining and immunolabeling it was validated that despite heterogeneous tumor composition, all tumors had a mitotic index of 20% or higher and that the major cell populations identified were present in multiple tumors (FIGS. 8A-8C).

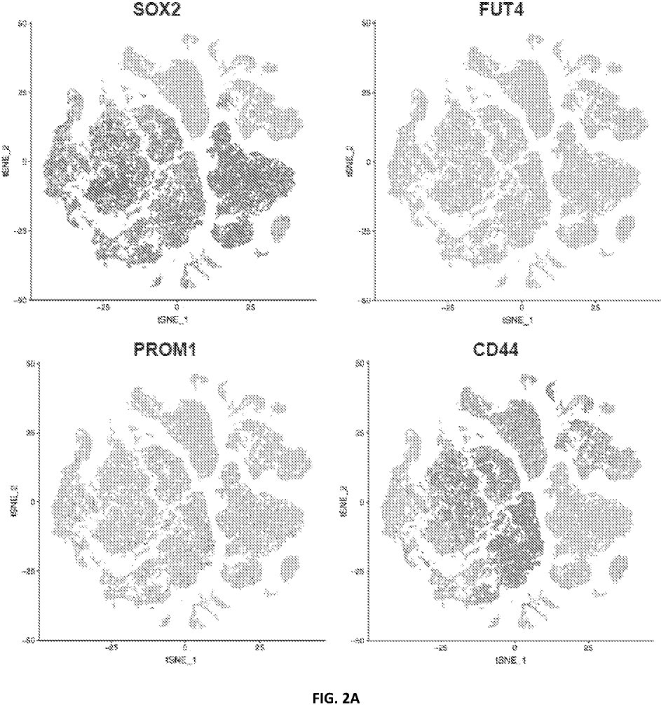

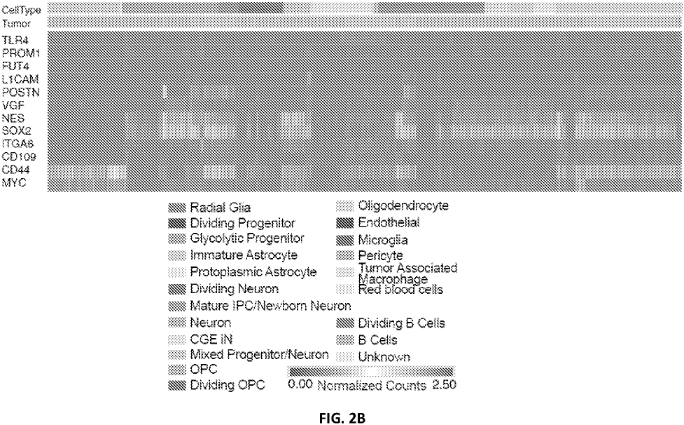

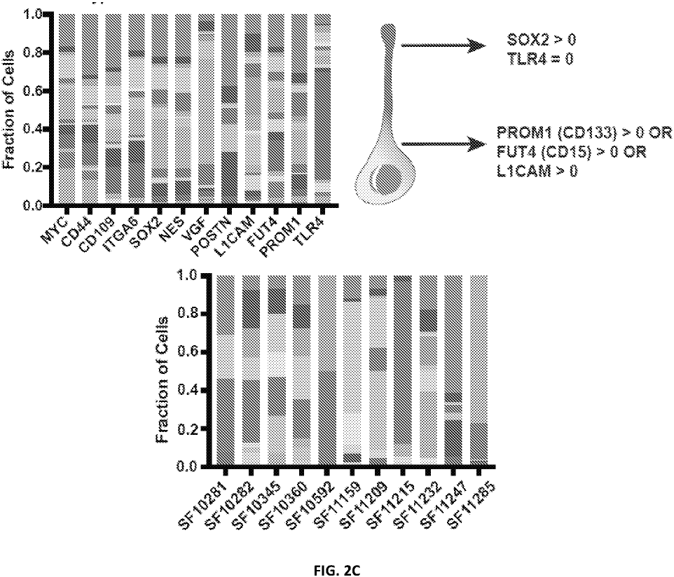

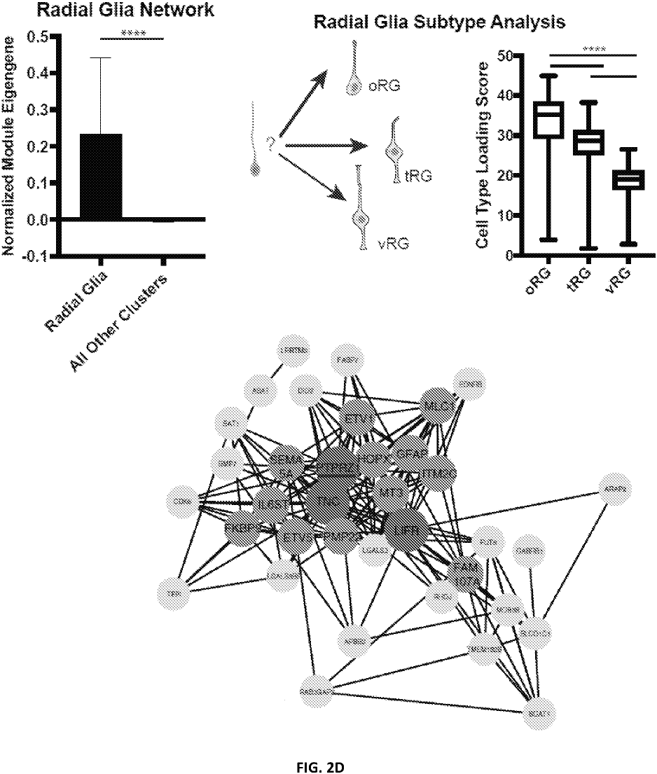

Glioblastoma stem cells (GSCs) are accepted as the cell population that gives rise to glioblastoma tumors, ultimately resulting in patient morbidity and mortality. GSCs were first identified through isolation of CD133+ cells from primary tumors, demonstrating that these cells were necessary and sufficient to give rise to an ectopic tumor. A number of other markers of GSCs were later identified and markers of progenitor identity such as SOX2 and NES have been used to validate the stemness of these GSC populations. The cell atlas of glioblastoma tumors was utilized to categorize the cell types that could be classified as GSCs based on the expression of commonly used GSC marker genes. Historically, protein expression of these stemness markers has been used as the gold-standard for the identity of cancer stem cell populations, and in the experiments described herein it was observed that at the RNA level there was widespread expression of these markers. Although distinct clusters of progenitor-like cell types including OPCs, radial glia, and intermediate progenitor cells were present, progenitor markers such as SOX2 and NES were expressed broadly (FIGS. 2A-2D). In contrast, PROM1 (CD133) and FUT4 (CD15), markers that have been shown to be sufficient to give rise to ectopic tumors, were very sparsely expressed. Almost every GSC marker gene could be found in a variety of cell types, with all 21 identified cell types expressing at least one marker associated with GSCs and stemness (FIGS. 2A-2D), though whether this transcriptional expression results in protein expression is unknown. Only VGF, a secreted neuropeptide that promotes survival and growth was substantially restricted, with the majority of its expression in OPCs. Other markers were expressed across cell types, including classic stem cell markers such as MYC and CD44, L1CAM, a marker that co-opts perivascular interactions, ITGA6, a regulator of GSC self-renewal identity, CD109, a marker of perivascular GSCs, and POSTN, a GSC secreted protein that recruits tumor associated macrophages that promote tumorigenesis (FIG. 2B). Although the expression of these transcripts does not guarantee that these proteins are expressed, they suggest that transcriptional programs associated with stemness are broadly expressed, and that activation of the stemness programs indicated by these GSC marker genes can occur in almost any cell type within the tumor.

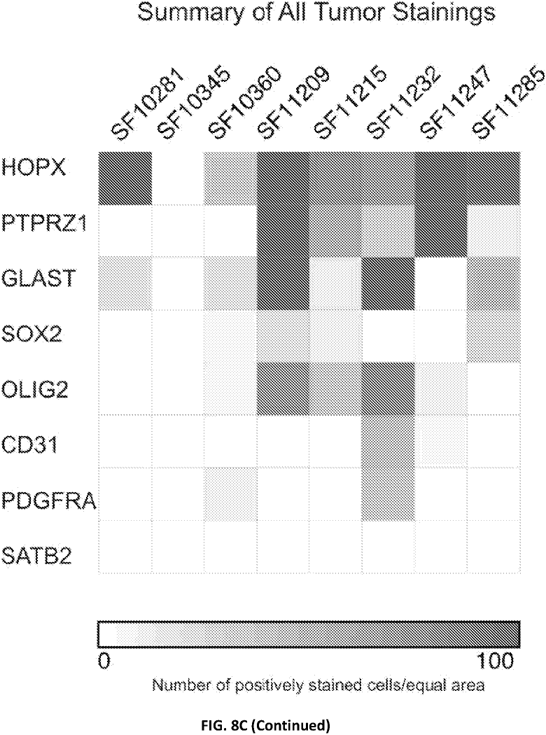

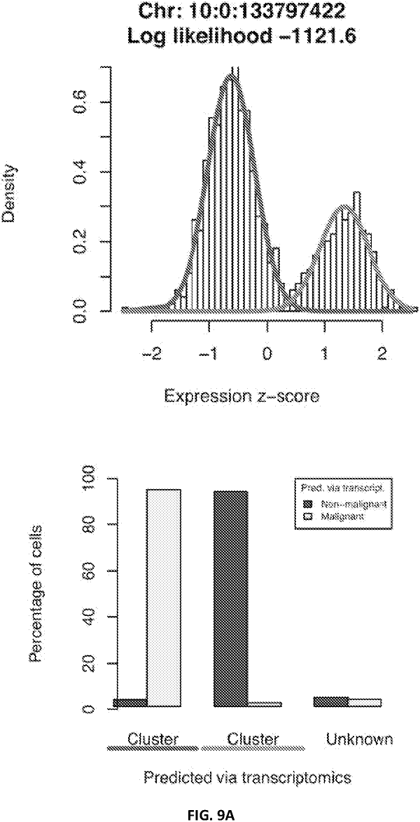

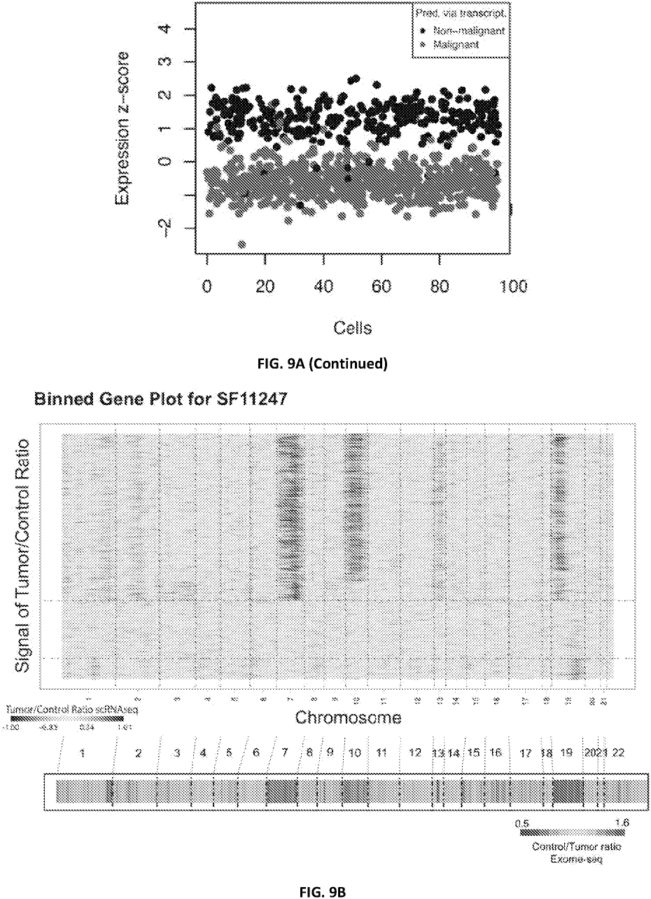

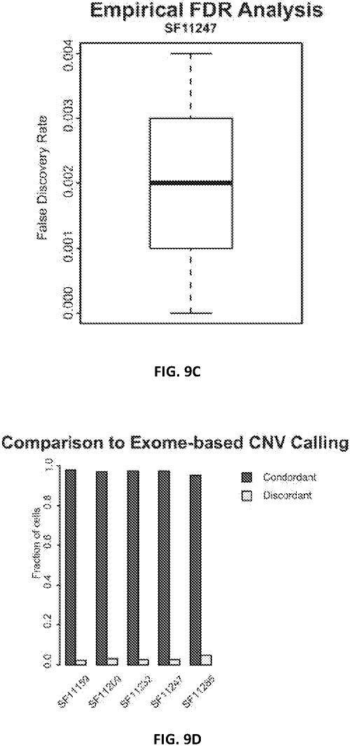

Models of glioblastoma tumor maintenance suggest that each tumor has a population of GSCs that interact with the tumor and the microenvironment to continually give rise to more tumor cells. To identify the potential GSC cell types for each tumor, GSCs were defined within the scRNA-seq dataset as any cell (independent of the previous clustering) expressing either PROM1, FUT4 or L1CAM, as well as SOX2, and not expressing TLR4 (which has been shown to be downregulated by GSCs to avoid immune surveillance). Each tumor expressed putative GSCs of at least two cell types, based upon our glioblastoma cell type atlas, with one tumor (SF11232) containing seven types of putative GSCs (FIG. 2C). Additionally, the combination of expressed genes that have previously been associated with GSC stemness was unique for each individual tumor (FIGS. 2A-2D). Thus, the cell types that make up glioblastomas and GSCs can be found in various combinations across tumors, but the exact cocktail of stemness markers (as defined by previous studies) co-expressed within a GSC cell type is largely specific to a single individual tumor. To test whether this stem cell heterogeneity could be inferred from previous studies the IVY glioblastoma repository was explored. Using IVY annotations of cell type markers as well as gene expression and tissue staining data for cancer stem cell markers, it was observed that individual tumors co-expressed a number of glioblastoma cell type markers, including a variety of GSC markers such as PROM1, FUT4, L1 CAM, and SOX2. Thus, a diverse set of GSCs can be found within a single tumor (FIGS. 9A-9C), characterized by heterogeneous marker gene combinations. Stemness programs are heterogeneous, and that multiple cell types may recruit stemness programs, even within a single tumor.

A number of previous studies have shown the potential for a variety of cell types to be transformed into GSCs. The experiments disclosed herein show representation of OPC, astrocyte, and neuronal cell types as GSCs. Additionally, radial glia emerged as a distinct cell type within the glioblastoma atlas that expressed GSC marker genes. Radial glia are stem cells of the developing human brain that give rise to neurons and glia in a temporal- and lineage-dependent fashion. However, radial glia are not believed to be present in the adult human brain and have not been identified in studies examining single-cell transcriptomes of the adult human brain suggesting that developmental programs are reactivated in the tumors. Radial glia have been hypothesized to play a role in other cancers such as ependymoma, and FABP7 (a marker of radial glia) expressing cells have been identified in glioblastoma. Several subtypes of, radial glia have recently been described in prenatal human development, including ventricular radial glia (vRG), outer radial glia (oRG) and truncated radial glia (tRG) based on distinct behavioral, morphological, and transcriptional signatures. Therefore, the similarity of the developmental transcriptional signatures of radial glia subtypes to radial glia-like tumor cells was explored. The highest correlation was found of radial glia-like tumor cell transcriptional profiles with the network signature of oRG cells. This network was strongly recapitulated in glioblastoma, with the same hub genes PTPRZ1, TNC, LIFR and strong connectivity between validated oRG markers including HOPX, FAM107A, and IL6ST (FIG. 2D).

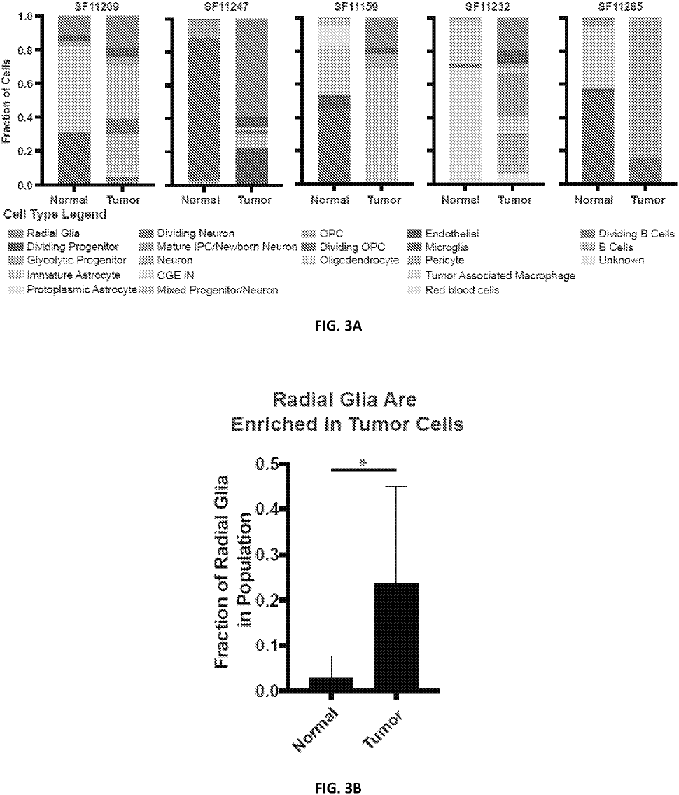

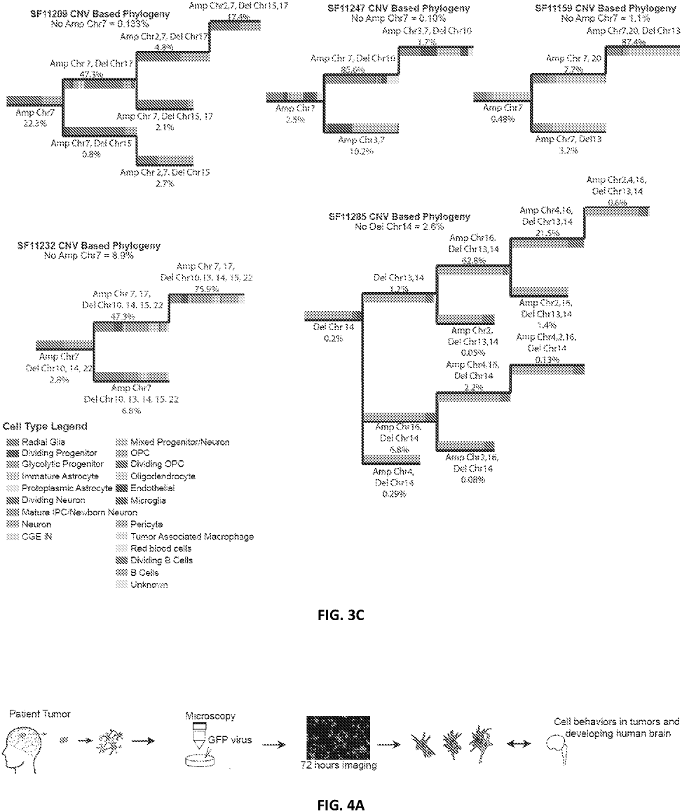

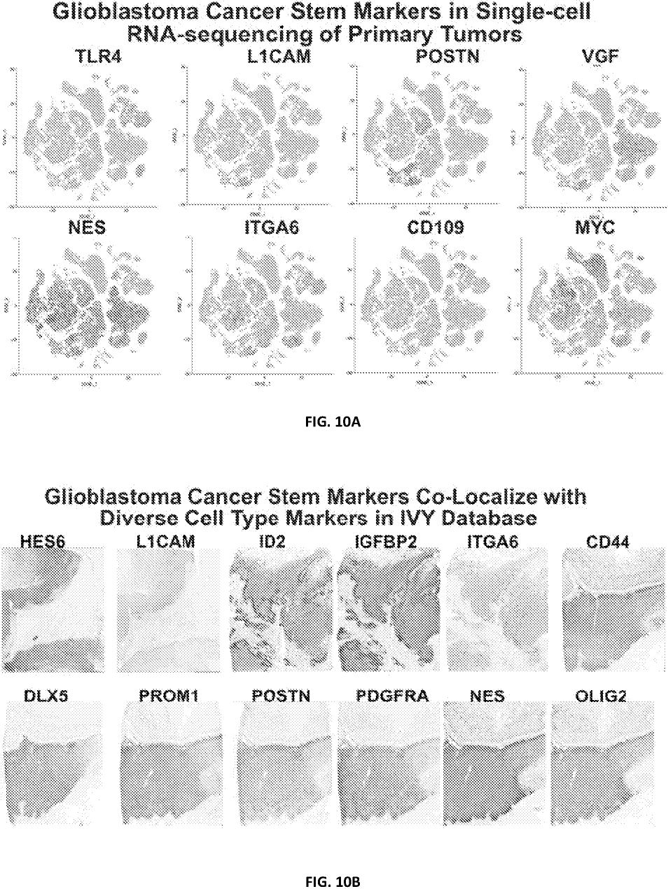

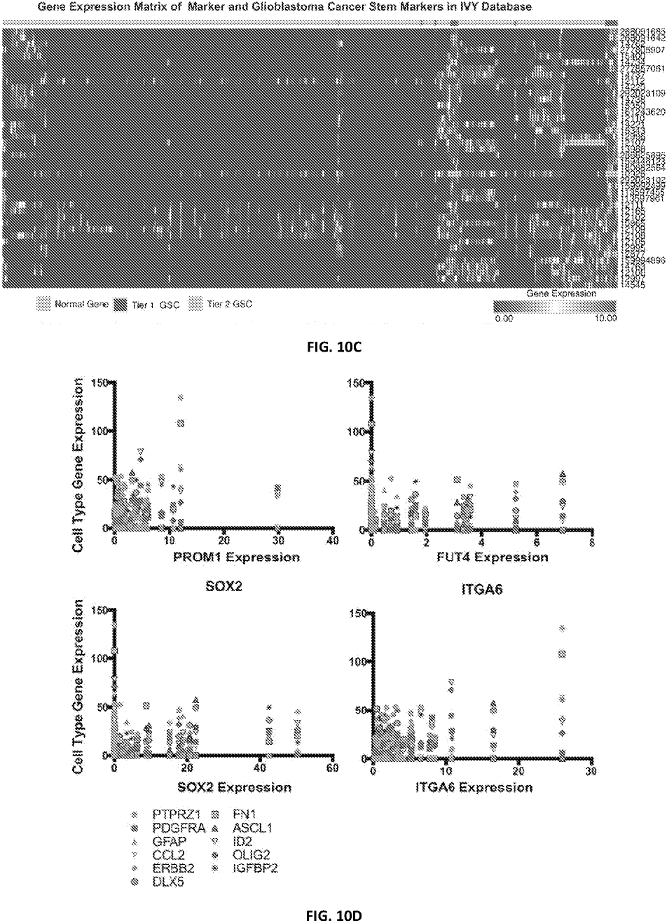

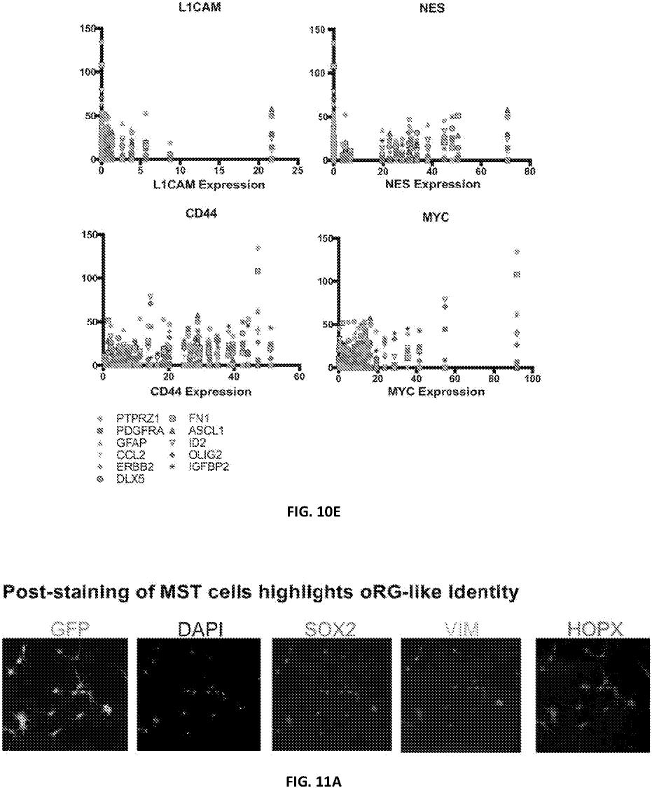

To model which of the GSC cell types contributes the most to tumors, cell type designations were used to empirically reconstruct the lineage relationship between cell populations in the tumor. Copy number variation (CNV) is a hallmark of tumorigenic cells and is less likely to be present in normal cells. To classify cells as belonging to malignant tumor or normal tissue, the presence/absence of somatic CNVs was assessed with CONICSmat. CNV analysis was performed on the five most deeply sequenced tumors based upon the transcriptomic data and was validated with exome sequencing and a variety of alternative methods, resulting in a low false-discovery rate (Materials and Methods, FIGS. 10A-10D). This analysis highlighted that immune lineage cells and differentiated cells were enriched in the populations of normal cells within the tumor, while progenitor or immature populations such as radial glia, OPCs, and immature astrocytes were enriched in tumorous cells (FIG. 3A and FIG. 10E). Based upon this CNV analysis, the most parsimonious lineage was empirically reconstructed between CNV events. By exploring the cell types associated with these CNV calls, the observations of heterogeneity are further supported; a variety of cell types can be observed at each arm of the phylogeny including dividing neurons, radial glia, and OPCs. Additionally, progenitor populations including oRG-like cells can be found along the lineage, suggesting that these progenitor populations may be maintaining their cell pool while producing additional tumor cell types. However, there is heterogeneity even at the base of these hierarchies, suggesting higher resolution analysis is required to further disentangle the lineage relationships between these presumptive source populations. These analyses indicate that radial glia-like glioblastoma cells, along with other progenitor populations, may serve as tumor propagating cells in glioblastoma.

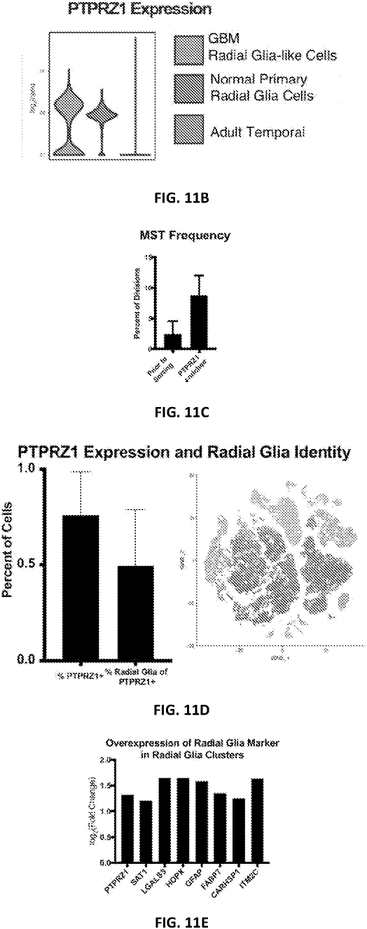

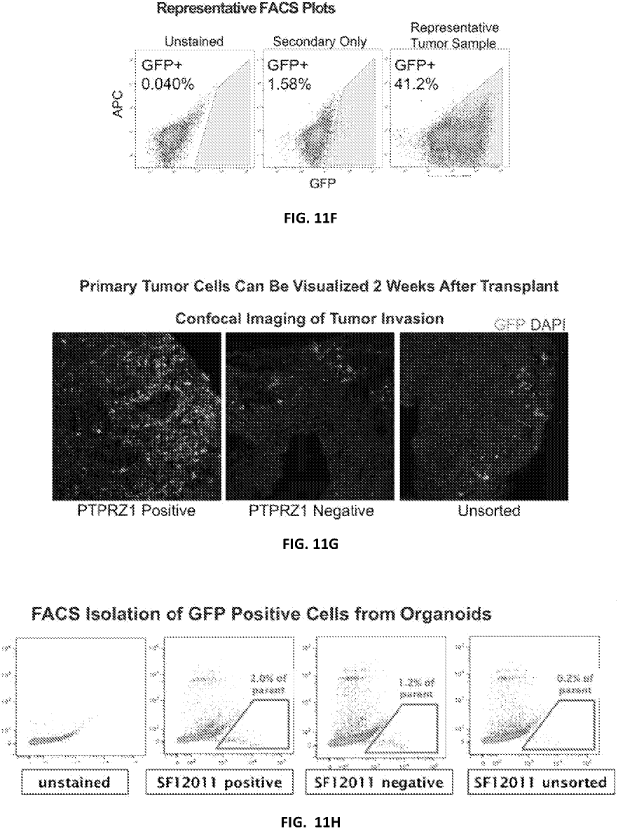

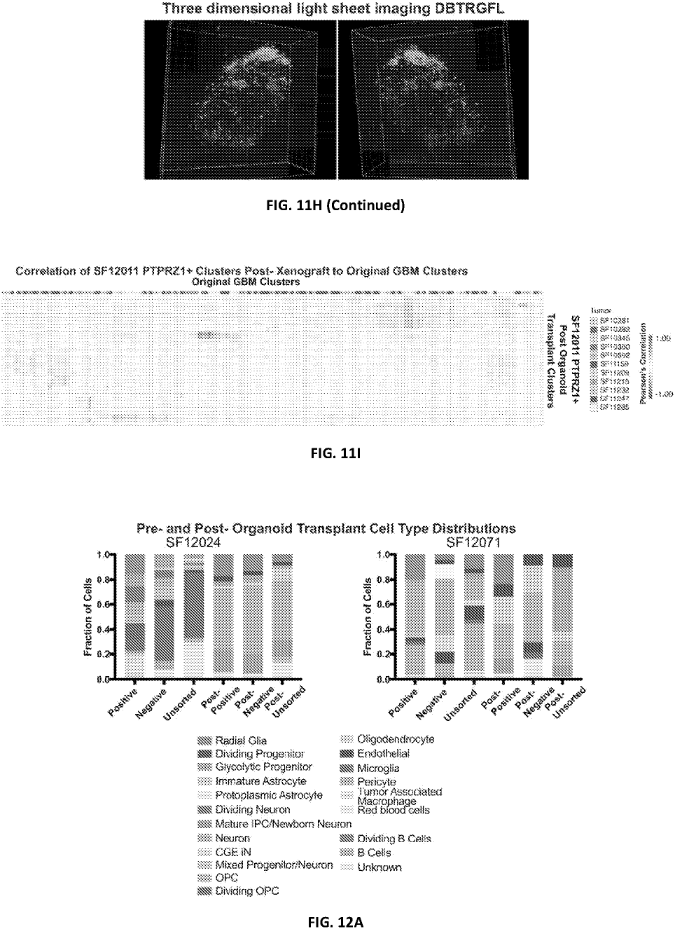

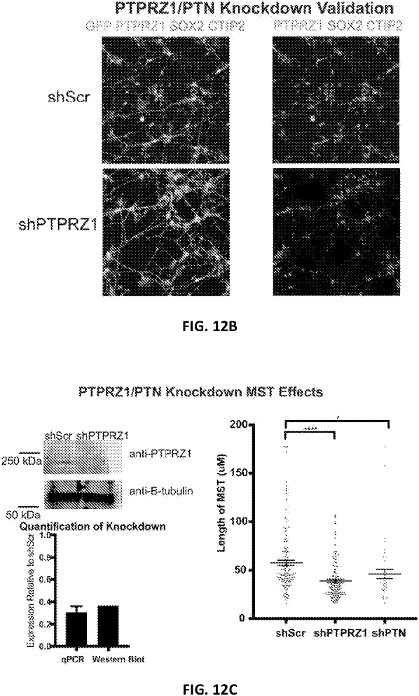

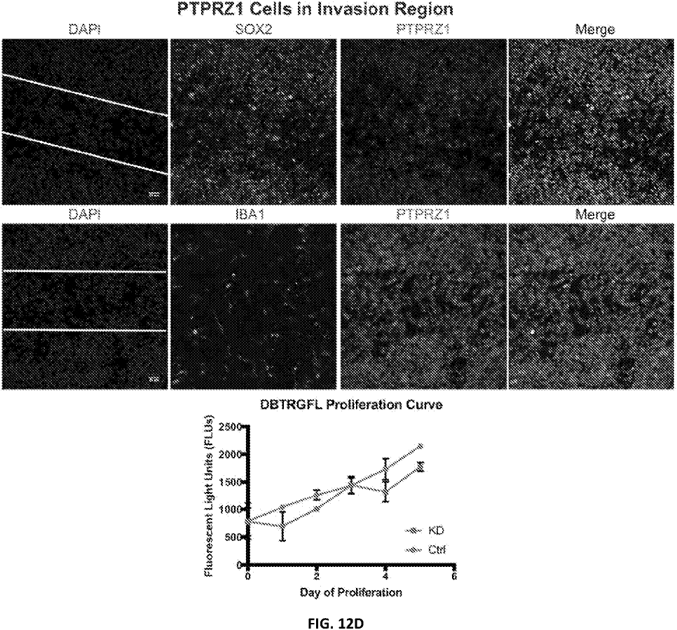

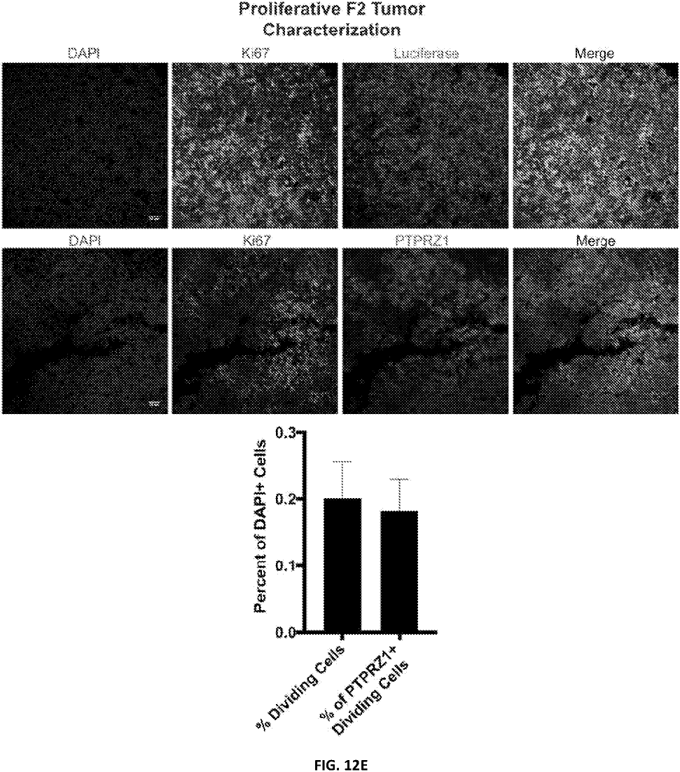

Outer radial glia cells were initially identified in the developing human brain based on their abundance within the outer subventricular zone and their unique characteristic mitotic behavior. They undergo mitotic somal translocation (MST), whereby the cell body translocates a distance of 50 to 100 µm prior to cytokinesis. The function of MST is hypothesized to play an important role in establishing an expanded neural stem cell niche in the developing cortex of large brain mammals. There has long been interest in understanding the cell behavior of glioblastoma cells. Because oRG signatures were observed in a subpopulation of glioblastoma cells, time-lapse imaging was used of dissociated cells from freshly resected tumors to explore glioblastoma cell behavior (FIG. 4A). Characteristic MST cell behavior was observed in a subset of cells from glioblastoma tumors (n = 5 tumors from different individuals) (FIG. 4B). Glioblastoma cells displayed similar somal translocation distances to their human developmental counterparts (FIG. 4C), and like prenatal oRG cells, they predominantly divided with a cleavage plane at right angles to the primary process, although a higher number of vertical and oblique divisions were observed in glioblastoma cells than in normal development (FIGS. 4D-4E). Post-staining of cells demonstrating MST behavior confirmed that these cells expressed markers of oRG cells (FIG. 11A). This suggests that re-expression of the developmental oRG signature in GSCs is associated with dynamic cell behavior characteristic of prenatal oRG progenitor cells. During development, oRG cells give rise to a transit amplifying progenitor cell population. To determine whether oRG-like glioblastoma cells could serve a similar role in glioblastoma, time-lapse imaging was used to trace the fate of daughter cells following oRG cell divisions. Instances were observed in which the daughter cells of oRG-like cell divisions also divided (FIG. 4F), demonstrating that this cell type is capable of amplifying the proliferative cell population in vitro. This model of amplification and differentiation resembles a pattern previously described in gliomas, where cancer stem cells generate daughter cells that subsequently differentiate into tumor bulk cells. During brain development, MST behavior enables the stem cell niche to expand during neurogenesis and gliogenesis, and the oRG-like MST events may play a similar role in glioblastoma expansion and invasion.

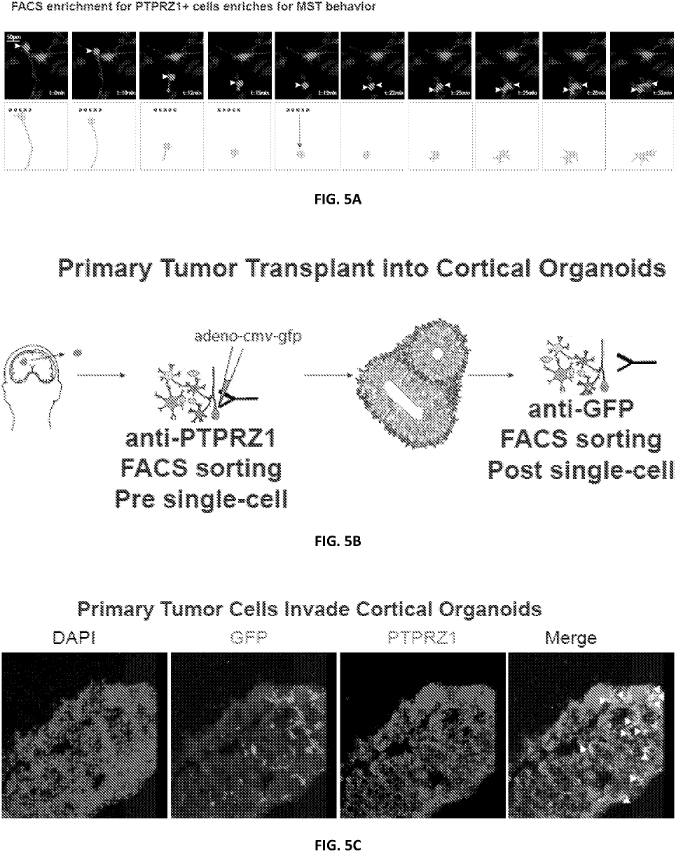

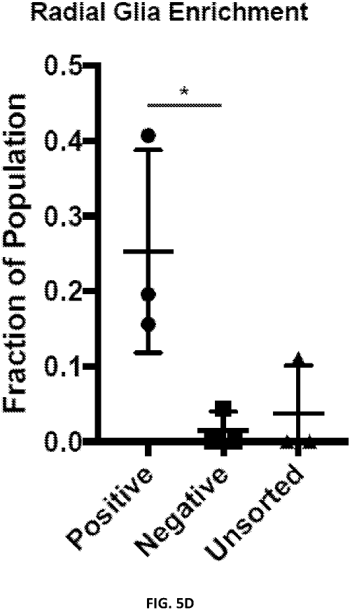

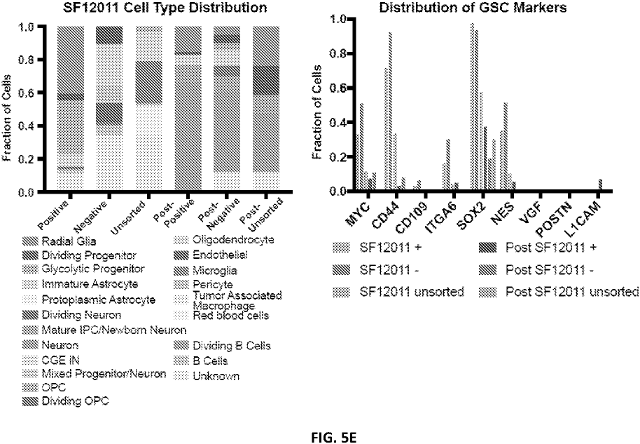

To functionally characterize the oRG-like population in glioblastoma cells, these cells were enriched using fluorescence-activated cell sorting (FACS). PTPRZ1 has been identified as a cell surface marker of the oRG cell population and is a hub gene in the conserved oRG gene network in glioblastoma. PTPRZ1-positive cells purified with FACS from fresh glioblastoma samples displayed MST behaviors, suggesting presence of the oRG-like cell population in the PTPRZ1-positive population (FIG. 5A and FIG. 11C). The role was characterized of PTPRZ1 positive oRG-like glioblastoma cells in tumor formation in the context of a human tissue microenvironment by transplanting PTPRZ1-sorted primary tumor cells into human cortical organoids (FIG. 5B).

Cortical organoids are a model of brain development that can be generated from human induced pluripotent stem cells and contain many of the major cell types present in the developing human brain. Organoids can promote development of cell heterogeneity and the establishment of a tumor niche, which may enable observations and screening approaches that are more difficult to recapitulate in mouse or two-dimensional models. Organoids have proved useful in the study of colorectal, pancreatic, and hematopoietic cancers, among others. To model glioblastoma, oncogenic events have been introduced into cortical organoids to create glioblastoma-like cell types. Though like genetic mouse models of cancers, it remains unclear how closely the tumorigenic trajectories and resultant cell types resemble patient cancers. This is of particular concern in a highly heterogeneous tumor such as glioblastoma. These structures maintain some aspects of glioblastoma cancer stem cell biology, but slow in their growth rate over time. Other groups have co-cultured or fused stable lines or spheroids from glioblastoma samples with cortical organoids. This approach maintains longer term cultures, and mimics aspects of cancer - normal cell interactions, but may create a selection bias for tumor cells that are more likely to generate stable lines.

In this disclosure, several existing approaches were merged by taking primary tumors directly from surgical resections, dissociating them, labeling them with an adenovirus, and subsequently transplanting them into cortical organoids derived from unmodified pluripotent stem cell lines. This label-based approach from freshly resected specimens results in a 100% engraftment rate, enables tracking of tumor cells, and allowed for lineage tracing - like approaches when paired with single-cell sequencing before and after transplantation. This approach enabled the observation that the diversity of cancer stem cells from the bulk tumor are well represented in the glioblastoma transplanted organoids, and that tumorigenic trajectories can be recapitulated from subsets of cancer stem cell-like populations.

Primary tumor samples were first sorted to isolate PTPRZ1 positive cells (FIG. 11F) for subsequent scRNA-seq and transplantation experiments. By scRNA-seq, the initial PTPRZ1 positive sorted populations were not uniform for a single cell type, consistent with prior experiments that have noted appreciable heterogeneity in populations previously thought to be homogenous and the observations of cell type heterogeneity among GSCs. However, the PTPRZ1 positive population was significantly enriched for radial glia-like cells compared to the PTPRZ1 negative population (FIG. 5D). To perform organoid transplants, PTPRZ1 positive, negative, and unsorted cell populations were first labeled with a GFP-expressing adenovirus. The cells were transplanted into cortical organoids at stages of neurogenesis, similar to published transplantation experiments. The tumor cells engrafted into the organoids, as visualized by GFP positive cell staining (FIG. 5C). Clearing of the organoid and lightsheet imaging showed GFP positive cells migrating into the organoid (FIG. 11G), suggesting that the approach enabled tumor cells to invade and expand within the organoid. GFP positive cells were isolated after two weeks (FIG. 11H) and again performed scRNA-seq.

Two weeks after transplantation of PTPRZ1 positive, negative, and unsorted cells, the tumor cell populations were composed primarily of either neuronal or astrocytic cells. The similar cell composition of the initial unsorted tumor and the PTRPZ1 enriched post-transplant tumor demonstrated the ability of PTPRZ1 positive cells to give rise to the diversity of cell types originally present in the bulk tumor (FIGS. 5B-5D). Interestingly, the cell types that were derived from the PTPRZ1 negative cells after organoid transplant also strongly resembled those that existed in the parallel unsorted populations, and also were highly correlated to the original glioblastoma clusters identified from the sequencing of 11 patients (FIGS. 11I and 12A). The similarity of the populations produced by PTPRZ1 positive and negative cells was further investigated by exploring the expression of canonical GSC markers in each of these populations. Both PTPRZ1 positive and negative sorted cells expressed GSC markers, consistent with the earlier observation that heterogeneous cell types express stemness markers in glioblastoma (FIGS. 5E and 12D). Interestingly, the expression of GSC markers uniformly decreased after transplant, which coincided with an increase in differentiated cell types within each population.

To better understand the molecular mechanism of PTPRZ1 signaling in glioblastoma tumor propagation, and to characterize how these cells differ from normal radial glia, in vitro KD was performed of PTRPZ1 in dissociated cell culture of primary developing cortex, primary tumor dissociated cell culture, and PDX dissociated cell culture. After infection with a scrambled hairpin control or shPTPRZ1 virus, cells were collected and single-cell RNA sequencing was performed. Each control/treatment pair was analyzed individually and the radial glia populations were isolated for further examination (FIG. 5F). Across both primary and glioblastoma samples, a significant overlap of PTPRZ1 regulated genes was identified. Interestingly, in addition to the targeted PTRPZ1 KD, other key markers of radial glia identity were also down-regulated, suggesting that PTPRZ1 signaling is required in determination of radial glia identity, and this manifested in a decrease in the proportion of both radial glia and other progenitor populations within each cell population sampled. Additionally, genes that are specifically down-regulated in glioblastoma but not in primary cells upon KD of PTPRZ1 relate to cholesterol and alcohol biosynthesis (FIG. 5F) which have been linked to the invasion of glioblastoma.

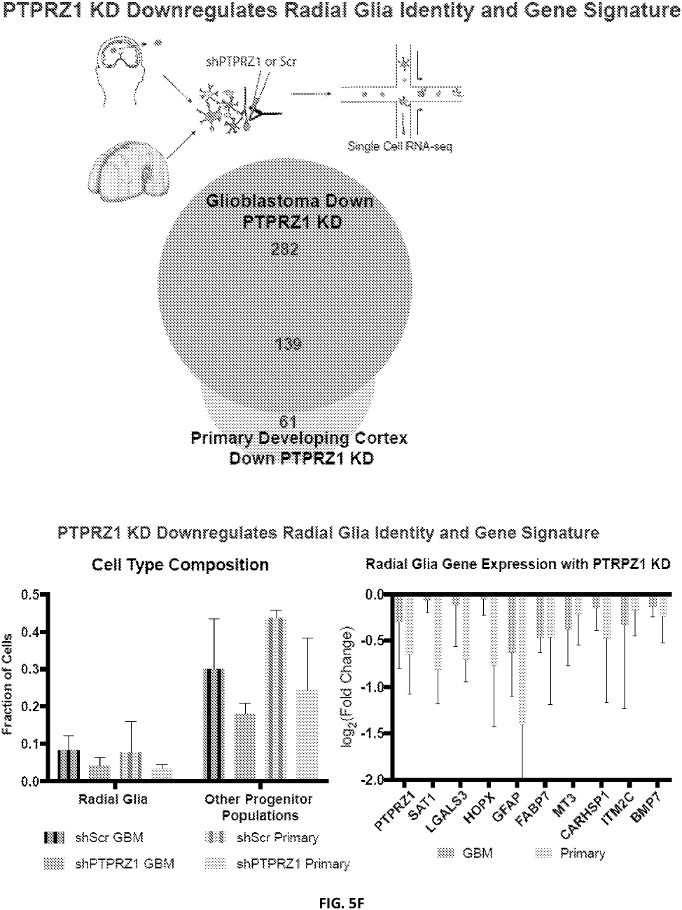

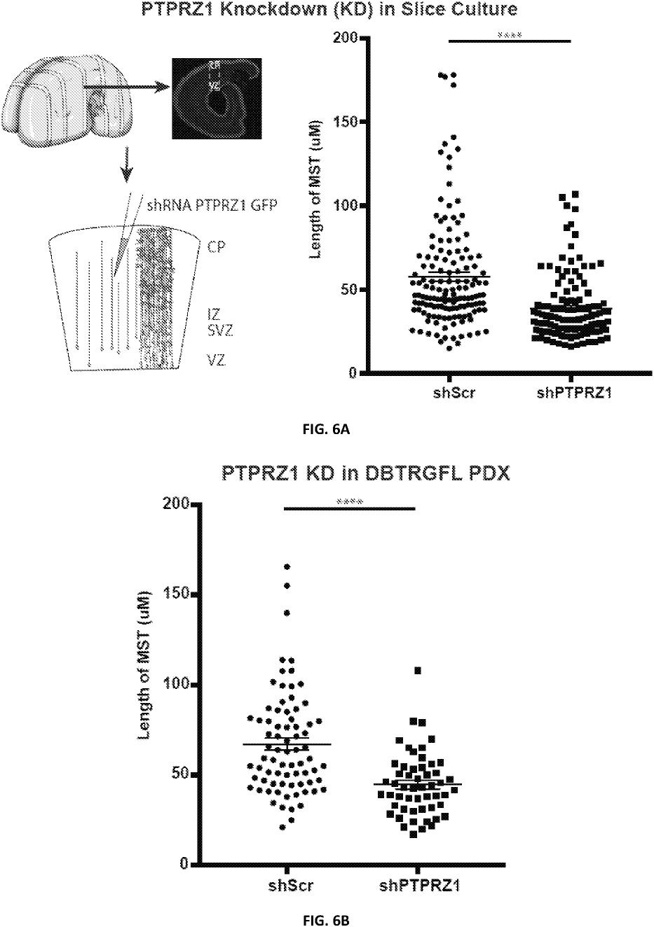

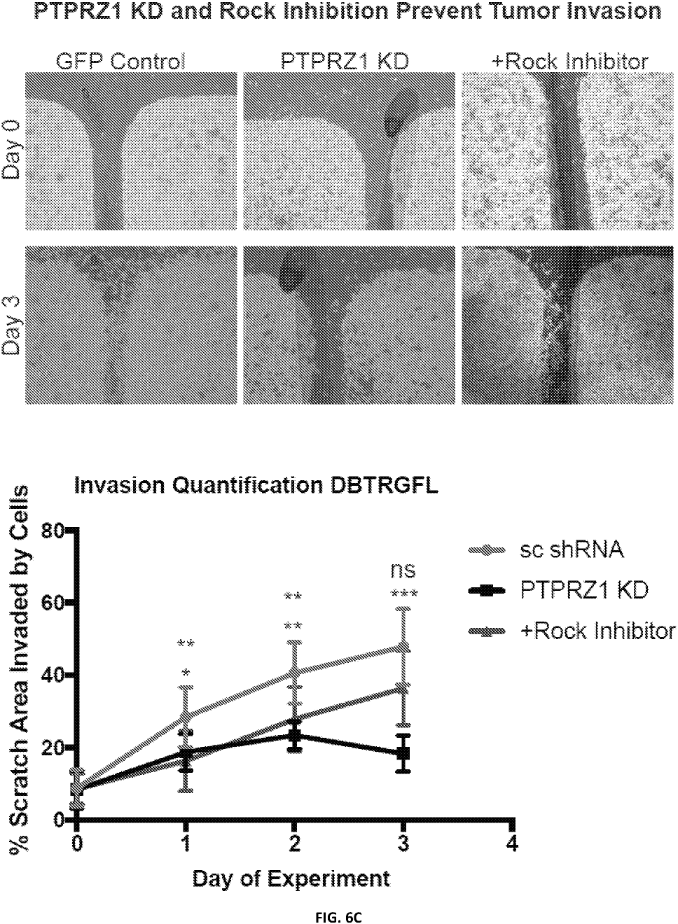

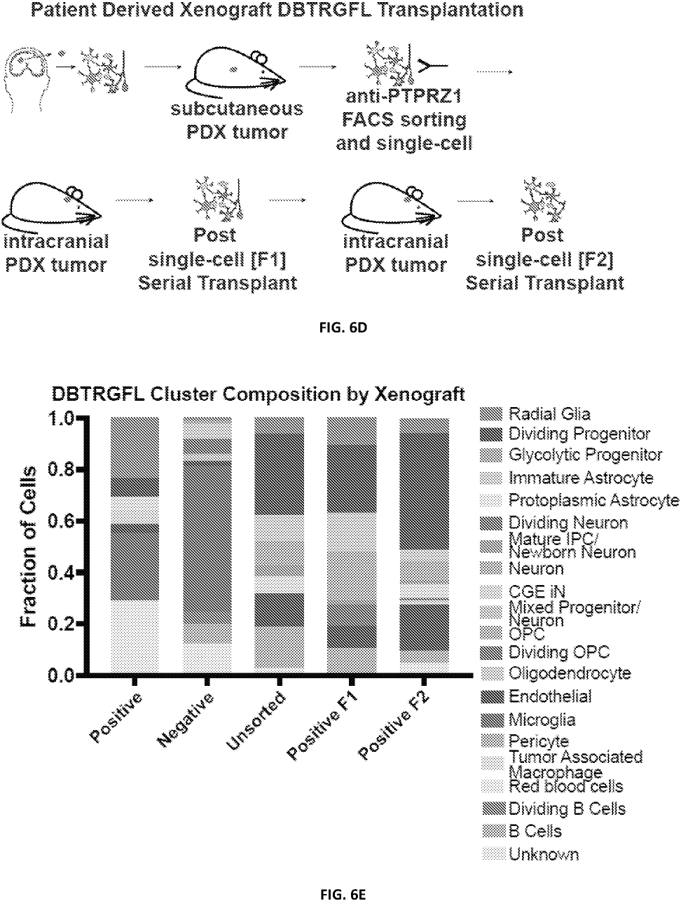

PTPRZ1 and its ligand, PTN, are identified in this disclosure as necessary for tumor invasion and viability. It was tested whether the expression of PTPRZ1 is required for the role of oRG-like cells in glioblastoma invasion using genetic and pharmacologic approaches. Previous work has linked PTPRZ1 and PTN with the Rho/Rho-kinase (ROCK) signaling pathway in glioblastoma. This was particularly intriguing as the ROCK pathway has been shown to be necessary for MST in human oRG cells during development, suggesting that PTPRZ1 may play a role in tumor invasion by oRG-like cells by mediating MST behavior. Given that the functional significance of key oRG markers such as PTPRZ1 remains unknown, and the role of MST has not been previously described in glioblastoma, the role of PTPRZ1/PTN signaling in oRG cell behavior was studied. shRNA mediated knock down of both PTPRZ1 and PTN in oRG cells was used during normal human development and the impact on MST was monitored. Knockdown of PTPRZ1 or PTN significantly reduced the length of MST (FIGS. 6A, 12C). Knockdown of PTPRZ1 similarly impacted the length of MST in the PDX line. To relate these findings to invasive behavior, in vitro invasion assays were performed on PDX tumor samples treated with either control, scrambled shRNAs, PTPRZ1 shRNAs, or Rock inhibitor to inhibit MST. Both PTPRZ1 KD and Rock pathway inhibition significantly decreased the invasive behavior of the PDX tumors (FIGS. 6C, 12D), suggesting a correlation between the inhibition of MST and a decrease in invasion.

### Example 2 - Identification of PTPRZ Expressing Cells as Mediators of Cancer Invasion

To explore the tumor propagation capabilities of PTPRZ1 + cells in vivo, PTRPZ1 positive cells were isolated from an existing luciferase labelled PDX line, DBTRGFL, and equal numbers of these cells were injected intracranially into the brains of immunocompromised mice. When tumors grew from the PTPRZ1 positively selected cells, serial transplantation of the PTRPZ1 positive cell derived tumors were additionally performed, paired with single-cell RNA sequencing of each set of tumors (first tumor F1, second tumors F2) (FIGS. 12E-12F). Comparing the PTRPZ1 positively sorted cells to the unsorted population prior to injection, an enrichment of radial glia-like cells was found. In both the F1 and F2 tumors, luciferase positive tumor cells consisting particularly of astrocytes and neurons that did not exist in the original sort were identified (FIGS. 6D-6E). Moreover, examination of the brain tissue surrounding the F2 tumor showed luciferase positive cells, a subset of which expressed PTRPZ1 (FIG. 12E), indicating that the F2 tumors invaded the surrounding brain tissue and that oRG-like PTPRZ1 positive cells can promote tumor invasion. Together, these results underscore the highly invasive nature of PTPRZ1-positive oRG-like glioblastoma cells and support their ability to propagate the tumor as one of several GSC cell types.

This disclosure identifies a heterogeneity of GSC cell types within and across glioblastomas. By comparison with data from the developing and adult human brain, an atlas was created of glioblastoma cell types and states. Although the exact composition of glioblastomas varies among individuals, similarities in cellular composition exist. Each tumor includes a set of proliferating, differentiated, and immune populations, and the exact mixture of these cell types creates a patient-specific cell type composition of each tumor. Orthogonal to this observation, GSC marker genes are enriched in a variety of cell types. The prevailing models of cancer stem cell biology purport that a single stem cell can initiate and give rise to the remaining tumor, and that this cell emerges from a single cell of origin. To prove the existence of a cancer stem cell under this model, limited dilution studies are the gold standard. The experiments disclosed herein indicate patterns that suggest the cancer stem cells in glioblastoma are more complicated. First, multiple cell populations within a single tumor have characteristics of cancer stem cell-like populations. Indeed, two (or possibly more) distinct populations are capable of giving rise to heterogeneous tumor populations. Moreover, glioblastomas have difficulty engrafting into mice and only a fraction of attempted primary tumors succeed in engrafting, but these engraftment rates increase substantially in cortical organoids. These findings support a role for relevant tumor niches that are required for promoting tumor growth, and for potential interactions between stem-cell like populations. In this model, terminology such as “cancer stem cell-like” may be more appropriate to suggest progenitor populations with unknown lineage relationships to one other, unknown potential to give rise to some or all other tumor populations, and that may require very narrow conditions to be conducive to proliferation.

The genes that make up each stemness program are very heterogeneous between tumors, indicating that stem cell markers can be expressed by multiple cell types in various combinations. However, these signatures are often very similar between cell types within a tumor. It is likely that the cell type composition of a tumor derives directly from the subtype of GSCs contained within each tumor. For example, the main cell type of GSC in tumor sample SF11215 is a dividing OPC cell, and more than 30% of the cell type composition of the tumor consists of oligodendrocytes, suggesting that the oligodendrocytes are generated from the dividing OPC-like GSCs. In other cases, the cell type composition of the tumor is much more heterogeneous and corresponds to many more GSC cell types. A radial glia-like GSC is identified that contributes to the cellular composition and invasive behavior of glioblastoma. During human cortical development, radial glia are the neural stem cells that generate the majority of neurons and glia and also give rise to transit amplifying populations. While radial glia are not believed to be present in the normal adult human brain, it is possible that there is a latent or quiescent population that can give rise to GSCs and glioblastoma, or that a neuronal or glial cell de-differentiates into a oRG-like cell to initiate tumors. It is possible the GSCs in glioblastoma, even when heterogeneous, arise from a single progenitor, or multiple lineages may coexist derived from one or more cells of origin. While the exact relationship among GSCs in glioblastoma is unknown, the secretion of factors, such as POSTNthat promote a proliferative niche may support crosstalk between GSC populations. Identifying GSCs based on their gene signatures is a first step toward characterizing cellular vulnerabilities that might be exploited for therapeutic purposes.

Culturing and expanding primary GSCs is essential to experimental characterization and manipulation of the cancer forming cell populations. However, performing xenografts and primary cell culture of glioblastoma has been challenging, largely because a vanishingly small proportion of primary tumors engraft into mice or adhere in vitro. These limitations are possibly related to difficulty replicating intracellular interactions that maintain glioblastoma tumor growth in vivo. Recent advances in the use of cortical organoid models as a substrate for glioblastoma expansion and culture may enable the study of primary tumors that often do not efficiently xenograft in mice. While the reasons for effective engraftment are not entirely understood, these models permit multiple primary tumors to be sorted, experimentally manipulated, and lineage traced over time. Using this model, the ability was demonstrated of oRG-like glioblastoma cells marked by PTPRZ1 to give rise to the differentiated cell types of the initial tumor. This model was used to validate the observations from single-cell sequencing that multiple GSC populations can exist within a single tumor, as the fraction depleted of oRG-like cells also gave rise to cell types not present in the initial sorted population. The application of organoids as tumor allografts may enable further functional analysis of GSC populations as well as provide a platform for drug testing.

“Reactivation” of the quintessential behavior of neural stem cells, mitotic somal translocation (MST) is described in glioblastoma. As this phenomenon is not known to occur outside of development, targeting MST may present a novel therapeutic strategy to cripple oRG-like GSCs. Additionally, characterizing this cell type highlights a potential link between previously described PTPRZ1 positive cell populations that mediate tumor invasion and the oRG-like cell. Although oRG-like cells represent one of several GSC cell types in primary glioblastoma tumors, oRG-like MST behavior may account for some aspects of tumor invasion and the oRG-like cells may also give rise to the differentiated neuronal and astrocytic cell types in glioblastoma tumors. The MST behavior is intriguing considering the aggressive invasive nature of glioblastoma tumors; even after surgical resection, glioblastomas frequently recur at sites distant to the initial tumor. Within the heterogeneity of GSCs in a single glioblastoma tumor, the spatial and temporal roles for each of the GSC populations may be specialized, or they may all act in parallel to promote tumorigenesis.

The preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

We claim:

1. A method for culturing primary cancer cells obtained from a subject, the method comprising:
implanting the primary cancer cells into a brain organoid generated from an embryonic stem cell (ESC) or an induced pluripotent stem cells (iPSC), wherein the ESC or iPSC is not genetically modified to render it oncogenic;
culturing the brain organoid comprising the implanted primary cancer cells for a period of at least one week,
wherein the implanted primary cancer cells remain viable for at least one week.

2. The method of claim 1, wherein the primary cancer cell is present in a composition comprising a population of cells isolated from a tumor tissue obtained from the subject and wherein the implanting comprises implanting the population of cells.

3. The method of claim 1 or 2, wherein the method comprises resecting a tumor and dissociating the tumor to provide dissociated primary cancer cells.

4. The method of any one of claims 1-3, wherein the primary cancer cell is obtained from a tumor, wherein the tumor comprises brain tumor, liver tumor, lung tumor, breast tumor, bone tumor, kidney tumor, prostate tumor, ovary tumor, or colon tumor.

5. The method of any one of claims 1-3, wherein the primary cancer cell is isolated from a cancer of a nervous system.

6. The method of claim 5, wherein the cancer of the nervous system is brain stem glioma, pineal astrocytic tumor, pilocytic astrocytoma, diffuse astrocytoma, anaplastic astrocytoma, glioblastoma, oligodendroglial tumor, mixed glioma, ependymal tumor, medulloblastoma, pineal parenchymal tumor, meningeal tumor, germ cell tumor, or craniopharyngioma.

7. The method of any one of claims 1-6, comprising determining growth of cancer cells in the organoid at least one week post-implantation.

8. The method of any one of claims 1-7, comprising determining the invasiveness of cancer cells in the organoid at least one week post-implantation.

9. The method of claim 7 or 8, wherein the determining comprises assessing cells expressing a marker specific for the cancer cells.

10. The method of any one of claims 1-9, wherein the brain organoid is generated from human embryonic stem cells (ESCs).

11. The method of any one of claims 1-9, wherein the brain organoid is generated from human induced pluripotent stem cells (iPSCs).

12. The method of any one of claims 1-9, wherein the brain organoid is generated from a cell line comprising human induced pluripotent stem cells (iPSCs).

13. The method of any one of claims 1-9, wherein the human ESCs or the human iPSCs are not genetically modified.

14. The method of any one of claims 1-13, wherein the brain organoid is a cortical organoid.

15. The method of claim 14, wherein the cortical organoid is generated by a method comprising:
culturing human ESCs or human iPSCs in a medium comprising a Rho kinase inhibitor, a Wnt signal inhibitor and a TGFβ signal inhibitor on a low adhesion substrate to generate aggregates; and
culturing the aggregates in a medium comprising a Wnt signal inhibitor and a TGFβ signal inhibitor and lacking a Rho kinase inhibitor, on a low adhesion substrate to generate the cortical organoid.

16. The method of any one of claims 1-15, wherein the brain organoid expresses one or more of the telencephalon markers Foxg1 and Six 3.

17. The method of claim 15 or 16, wherein the Wnt signal inhibitor comprises IWR-1.

18. The method of any one of claims 15-17, wherein the TGFβ signal inhibitor comprises SB431542.

19. The method of any one of claims 15-18, wherein the Rho kinase inhibitor comprises Y-27632.

20. The method of any one of claims 1-19, wherein the primary cancer cell expresses protein tyrosine phosphatase receptor type Z1 (PTPRZ1).

21. The method of any one claims 1-20, further comprising screening a candidate agent for activity in reducing cancer cell growth and/or invasion, the method comprising:
culturing the primary cancer cells in the brain organoid in the presence of the candidate agent and culturing the primary cancer cells in the brain organoid
wherein reduced cancer cell growth of and/or invasion as compared to the cancer cell growth and/or invasion, respectively, in absence of the candidate agent identifies the candidate agent as having activity in reducing cancer cell growth and/or invasion.

22. The method of claim 21, wherein the candidate agent is a small molecule, oligonucleotide, peptide, or protein.

23. A brain organoid generated from an ESC or an iPSC, the brain organoid comprising implanted therein primary cancer cells derived from a cancer of a nervous system, and wherein the ESC or iPSC is not genetically modified to render it oncogenic.

24. The brain organoid of claim 23, wherein the ESC or iPSC is a human ESC or human iPSC.

25. The brain organoid of any of claims 23 or 24, wherein the primary cancer cell is isolated from a brain stem glioma, pineal astrocytic tumor, pilocytic astrocytoma, diffuse astrocytoma, anaplastic astrocytoma, glioblastoma, oligodendroglial tumor, mixed glioma, ependymal tumor, medulloblastoma, pineal parenchymal tumor, meningeal tumor, germ cell tumor, or craniopharyngioma.

26. The brain organoid of any one of claims 23 to 25, the brain organoid is a cortical organoid.

27. A method for inhibiting the invasion of a cancer of the nervous system, the method comprising administering to a subject suffering from the cancer of the nervous system an inhibitor of protein tyrosine phosphatase receptor type Z1 (PTPRZ1) pathway.

28. The method of claim 27, wherein the cancer is in the brain, spinal cord, or a neuroendocrine gland.

29. The method of claim 27 or claim 28, wherein the cancer is brain stem glioma, pineal astrocytic tumor, pilocytic astrocytoma, diffuse astrocytoma, anaplastic astrocytoma, glioblastoma, oligodendroglial tumor, mixed glioma, ependymal tumor, medulloblastoma, pineal parenchymal tumor, meningeal tumor, germ cell tumor, or craniopharyngioma.

30. The method of any one of claims 27-29, wherein the inhibitor of the PTPRZ1 pathway is an inhibitor of the expression or activity of PTPRZ1 or an inhibitor of the expression or activity of pleiotrophin (PTN).

31. The method of claim 30, wherein the inhibitor of PTPRZ1 expression is an oligonucleotide that specifically inhibits the transcription of PTPRZ1 mRNA or translation of PTPRZ1 protein.

32. The method of claim 30, wherein the oligonucleotide that specifically inhibits the transcription of PTPRZ1 mRNA is an interfering RNA (RNAi).

33. The method of claim 32, wherein the RNAi is small interfering RNA (siRNA), double-stranded RNA (dsRNA), single-stranded RNA (ssRNA), micro-RNA (miRNA), or short hairpin RNA (shRNA) that specifically inhibits the transcription of PTPRZ1 mRNA.

34. The method of claim 30, wherein the inhibitor of PTN expression is an oligonucleotide that specifically inhibits the transcription of PTN mRNA or translation of PTN protein.

35. The method of claim 34, wherein the oligonucleotide that specifically inhibits the transcription of PTN mRNA is an interfering RNA (RNAi).

36. The method of claim 35, wherein the RNAi is small interfering RNA (siRNA), double-stranded RNA (dsRNA), single-stranded RNA (ssRNA), micro-RNA (miRNA), or short hairpin RNA (shRNA) that specifically inhibits the transcription of PTN mRNA.

37. The method of claim 30, wherein the inhibitor of activity of PTPRZ1 is an antibody or an antigen binding fragment of an antibody that specifically binds to PTPRZ1, optionally, conjugated to a chemotherapeutic agent.

38. The method of claim 37, wherein the antibody is a monoclonal antibody, polyclonal antibody, human antibody, humanized antibody, chimeric antibody, a variable domain fragment, Fab fragment, Fab₂ fragment, Fab₃ fragment, Fab′ fragment, F(ab′)₂ fragment, Fd fragment, rlgG, dibody, tribody, tetrabody, minibody, monovalent antibody, divalent antibody, multivalent antibody, single chain variable fragment (ScFv), or bis-scFv that specifically binds to PTPRZ1.

39. The method of claim 30, wherein the inhibitor of activity of PTN is an antibody or an antigen binding fragment of an antibody that specifically binds to PTN, optionally, conjugated to a chemotherapeutic agent.

40. The method of claim 39, wherein the antibody is a monoclonal antibody, polyclonal antibody, human antibody, humanized antibody, chimeric antibody, a variable domain fragment, Fab fragment, Fab₂ fragment, Fab₃ fragment, Fab′ fragment, F(ab′)₂ fragment, Fd fragment, rlgG, dibody, tribody, tetrabody, minibody, monovalent antibody, divalent antibody, multivalent antibody, single chain variable fragment (ScFv), or bis-scFv that specifically binds to PTN.

41. The method of claim 30, wherein the inhibitor of PTPRZ1 or PTN expression or activity is a small molecule compound.

42. The method of any one of claims 27-41, comprising administering the inhibitor of the PTPRZ1 pathway specifically in and around the cancerous tissue of the subject.

43. The method of any one of claims 27-42, comprising packaging the inhibitor in a carrier that is specifically targeted to the cancer cells.

44. A method for culturing a PTPRZ1 expressing cancer cell, the methods comprising:
a) providing a brain organoid;
b) isolating, from a sample of cancer cells obtained from a subject, the cancer cell that express PTPRZ1,
c) transplanting the PTPRZ1 expressing cancer cell into the brain organoid, and
d) culturing the transplanted PTPRZ1 expressing cancer cell.

45. The method of claim 44, wherein the brain organoid is a neurosphere, neural aggregate, neural rossette, cortical spheroid, cortical organoid, cerebral organoid, or whole-brain organoid.

46. The method of claim 44 or claim 45, wherein the step of isolating the PTPRZ1 expressing cancer cell comprises disrupting a cancerous mass obtained from the subject, labeling the PTPRZ1 expressing cell with a marker, and isolating the cell labeled with the marker.

47. The method of claim 46, wherein the marker is as fluorescent labeled PTPRZ1 antibody and isolating comprises performing flow cytometry.

48. The method of any one of claims 44 to 47, further comprising, after isolating the PTPRZ1 expressing cancer cell but before transplanting the PTPRZ1 expressing cancer cell into the brain organoid, transfecting the PTPRZ1 expressing cancer cell with a polynucleotide encoding a label.

49. The method of any one of claims 44 to 48, wherein the PTPRZ1 expressing cancer cell is a PTPRZ1 expressing outer radial glia-like cell.

50. A method for identifying a candidate agent as an inhibitor of cancer cell invasion, wherein the cancer cell expresses PTPRZ1, the method comprising:
culturing a first PTPRZ1 expressing cancer cell in the presence of the candidate agent and a second PTPRZ1 expressing cancer cell in the absence of the candidate agent;
wherein reduced cancer cell of the first PTPRZ1 expressing cancer cell as compared to the second PTPRZ1 expressing cancer cells identifies the candidate agent as an inhibitor of cancer cell invasion.

51. The method of claim 50, comprising culturing the first and the second PTPRZ1 expressing cancer cells in a brain organoid.

52. The method of claim 51, comprising assessing the mitotic somal translocation (MST) of the first and the second PTPRZ1 expressing cancer cell and identifying the candidate agent as an inhibitor of cancer cell invasion if the first PTPRZ1 expressing cancer cell exhibits lower MST than that of the second PTPRZ1 expressing cancer cell.

53. The method of any one of claims 50-52, comprising transfecting the first and the second PTPRZ1 expressing cancer cells with a polynucleotide encoding a marker and transplanting the first PTPRZ1 expressing cancer cell into a first brain organoid, and the second PTPRZ1 expressing cancer cell into a second brain organoid prior to culturing the first PTPRZ1 expressing cancer cell in the absence of the candidate compound and culturing the second PTPRZ1 expressing cancer cell in the presence of the candidate agent.

54. The method of any one of claims 50-53, wherein the first and second PTPRZ1 expressing cancer cell is an oRG-like glioblastoma cell.

55. The method of claim 53 or claim 54, wherein the marker is green fluorescent protein, blue fluorescent protein, cyan-fluorescent protein, enhanced GFP with red-shifted excitation, enhanced yellow-fluorescent protein, or photoactivatable GFP.

56. The method of any one of claims 50 to 55, further comprising observing the MST of the first and the second PTPRZ1 expressing cancer cells and identifying the candidate agent as an inhibitor of cancer cell invasion if the first PTPRZ1 expressing cancer cell exhibits lower MST than that of the second PTPRZ1 expressing cancer cell.

57. The method of any one of claims 53 to 56, wherein the nucleotide encoding the marker comprises a double stranded linear or circular DNA vector.

58. The method of claim 57, wherein the DNA vector is a viral vector.

59. The method of claim 58, wherein the viral vector is an adenoviral vector, Simian Virus 40 (SV40) vector, polyomaviral vector, herpesviral vector, or papoviral vector.


SEQUENCE LISTING (6 sequences)

SEQ ID NO: 1 - protein, 2314 aa, Homo sapiens
MRILKRFLACIQLLCVCRLDWANGYYRQQRKLVEEIGWSYTGALNQKNWGKKYPTCNSPKQSPINIDEDLTQVNVNLKKLKFQGWDKTSLENTFIHNTGKTVEINLTNDYRVSGGVSEMVFKASKITFHWGKCNMSSDGSEHSLEGQKFPLEMQIYCFDADRFSSFEEAVKGKGKLRALSILFEVGTEENLDFKAIIDGVESVSRFGKQAALDPFILLNLLPNSTDKYYIYNGSLTSPPCTDTVDWIVFKDTVSISESQLAVFCEVLTMQQSGYVMLMDYLQNNFREQQYKFSRQVFSSYTGKEEIHEAVCSSEPENVQADPENYTSLLVTWERPRVVYDTMIEKFAVLYQQLDGEDQTKHEFLTDGYQDLGAILNNLLPNMSYVLQIVAICTNGLYGKYSDQLIVDMPTDNPELDLFPELIGTEEIIKEEEEGKDIEEGAIVNPGRDSATNQIRKKEPQISTTTHYNRIGTKYNEAKTNRSPTRGSEFSGKGDVPNTSLNSTSQPVTKLATEKDISLTSQTVTELPPHTVEGTSASLNDGSKTVLRSPHMNLSGTAESLNTVSITEYEEESLLTSFKLDTGAEDSSGSSPATSAIPFISENISQGYIFSSENPETITYDVLIPESARNASEDSTSSGSEESLKDPSMEGNVWFPSSTDITAQPDVGSGRESFLQTNYTEIRVDESEKTTKSFSAGPVMSQGPSVTDLEMPHYSTFAYFPTEVTPHAFTPSSRQQDLVSTVNVVYSQTTQPVYNGETPLQPSYSSEVFPLVTPLLLDNQILNTTPAASSSDSALHATPVFPSVDVSFESILSSYDGAPLLPFSSASFSSELFRHLHTVSQILPQVTSATESDKVPLHASLPVAGGDLLLEPSLAQYSDVLSTTHAASETLEFGSESGVLYKTLMFSQVEPPSSDAMMHARSSGPEPSYALSDNEGSQHIFTVSYSSAIPVHDSVGVTYQGSLFSGPSHIPIPKSSLITPTASLLQPTHALSGDGEWSGASSDSEFLLPDTDGLTALNISSPVSVAEFTYTTSVFGDDNKALSKSEIIYGNETELQIPSFNEMVYPSESTVMPNMYDNVNKLNASLQETSVSISSTKGMFPGSLAHTTTKVFDHEISQVPENNFSVQPTHTVSQASGDTSLKPVLSANSEPASSDPASSEMLSPSTQLLFYETSASFSTEVLLQPSFQASDVDTLLKTVLPAVPSDPILVETPKVDKISSTMLHLIVSNSASSENMLHSTSVPVFDVSPTSHMHSASLQGLTISYASEKYEPVLLKSESSHQVVPSLYSNDELFQTANLEINQAHPPKGRHVFATPVLSIDEPLNTLINKLIHSDEILTSTKSSVTGKVFAGIPTVASDTFVSTDHSVPIGNGHVAITAVSPHRDGSVTSTKLLFPSKATSELSHSAKSDAGLVGGGEDGDTDDDGDDDDDRDSDGLSIHKCMSCSSYRESQEKVMNDSDTHENSLMDQNNPISYSLSENSEEDNRVTSVSSDSQTGMDRSPGKSPSANGLSQKHNDGKEENDIQTGSALLPLSPESKAWAVLTSDEESGSGQGTSDSLNENETSTDFSFADTNEKDADGILAAGDSEITPGFPQSPTSSVTSENSEVFHVSEAEASNSSHESRIGLAEGLESEKKAVIPLVIVSALTFICLVVLVGILIYWRKCFQTAHFYLEDSTSPRVISTPPTPIFPISDDVGAIPIKHFPKHVADLHASSGFTEEFETLKEFYQEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLV

SEQ ID NO: 2 - DNA, 7941 bp, Homo sapiens
cacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctggattgggctaatggatactacagacaacagagaaaacttgttgaagagattggctggtcctatacaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgagatgcaaatctactgctttgatgcggaccgattttcaagttttgaggaagcagtcaaaggaaaagggaagttaagagctttatccattttgtttgaggttgggacagaagaaaatttggatttcaaagcgattattgatggagtcgaaagtgttagtcgttttgggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttgggtgctattctcaataatttgctacccaatatgagttatgttcttcagatagtagccatatgcactaatggcttatatggaaaatacagcgaccaactgattgtcgacatgcctactgataatcctgaacttgatcttttccctgaattaattggaactgaagaaataatcaaggaggaggaagagggaaaagacattgaagaaggcgctattgtgaatcctggtagagacagtgctacaaaccaaatcaggaaaaaggaaccccagatttctaccacaacacactacaatcgcatagggacgaaatacaatgaagccaagactaaccgatccccaacaagaggaagtgaattctctggaaagggtgatgttcccaatacatctttaaattccacttcccaaccagtcactaaattagccacagaaaaagatatttccttgacttctcagactgtgactgaactgccacctcacactgtggaaggtacttcagcctctttaaatgatggctctaaaactgttcttagatctccacatatgaacttgtcggggactgcagaatccttaaatacagtttctataacagaatatgaggaggagagtttattgaccagtttcaagcttgatactggagctgaagattcttcaggctccagtcccgcaacttctgctatcccattcatctctgagaacatatcccaagggtatatattttcctccgaaaacccagagacaataacatatgatgtccttataccagaatctgctagaaatgcttccgaagattcaacttcatcaggttcagaagaatcactaaaggatccttctatggagggaaatgtgtggtttcctagctctacagacataacagcacagcccgatgttggatcaggcagagagagctttctccagactaattacactgagatacgtgttgatgaatctgagaagacaaccaagtccttttctgcaggcccagtgatgtcacagggtccctcagttacagatctggaaatgccacattattctacctttgcctacttcccaactgaggtaacacctcatgcttttaccccatcctccagacaacaggatttggtctccacggtcaacgtggtatactcgcagacaacccaaccggtatacaatggtgagacacctcttcaaccttcctacagtagtgaagtctttcctctagtcacccctttgttgcttgacaatcagatcctcaacactacccctgctgcttcaagtagtgattcggccttgcatgctacgcctgtatttcccagtgtcgatgtgtcatttgaatccatcctgtcttcctatgatggtgcacctttgcttccattttcctctgcttccttcagtagtgaattgtttcgccatctgcatacagtttctcaaatccttccacaagttacttcagctaccgagagtgataaggtgcccttgcatgcttctctgccagtggctgggggtgatttgctattagagcccagccttgctcagtattctgatgtgctgtccactactcatgctgcttcagagacgctggaatttggtagtgaatctggtgttctttataaaacgcttatgttttctcaagttgaaccacccagcagtgatgccatgatgcatgcacgttcttcagggcctgaaccttcttatgccttgtctgataatgagggctcccaacacatcttcactgtttcttacagttctgcaatacctgtgcatgattctgtgggtgtaacttatcagggttccttatttagcggccctagccatataccaatacctaagtcttcgttaataaccccaactgcatcattactgcagcctactcatgccctctctggtgatggggaatggtctggagcctcttctgatagtgaatttcttttacctgacacagatgggctgacagcccttaacatttcttcacctgtttctgtagctgaatttacatatacaacatctgtgtttggtgatgataataaggcgctttctaaaagtgaaataatatatggaaatgagactgaactgcaaattccttctttcaatgagatggtttacccttctgaaagcacagtcatgcccaacatgtatgataatgtaaataagttgaatgcgtctttacaagaaacctctgtttccatttctagcaccaagggcatgtttccagggtcccttgctcataccaccactaaggtttttgatcatgagattagtcaagttccagaaaataacttttcagttcaacctacacatactgtctctcaagcatctggtgacacttcgcttaaacctgtgcttagtgcaaactcagagccagcatcctctgaccctgcttctagtgaaatgttatctccttcaactcagctcttattttatgagacctcagcttcttttagtactgaagtattgctacaaccttcctttcaggcttctgatgttgacaccttgcttaaaactgttcttccagctgtgcccagtgatccaatattggttgaaacccccaaagttgataaaattagttctacaatgttgcatctcattgtatcaaattctgcttcaagtgaaaacatgctgcactctacatctgtaccagtttttgatgtgtcgcctacttctcatatgcactctgcttcacttcaaggtttgaccatttcctatgcaagtgagaaatatgaaccagttttgttaaaaagtgaaagttcccaccaagtggtaccttctttgtacagtaatgatgagttgttccaaacggccaatttggagattaaccaggcccatcccccaaaaggaaggcatgtatttgctacacctgttttatcaattgatgaaccattaaatacactaataaataagcttatacattccgatgaaattttaacctccaccaaaagttctgttactggtaaggtatttgctggtattccaacagttgcttctgatacatttgtatctactgatcattctgttcctataggaaatgggcatgttgccattacagctgtttctccccacagagatggttctgtaacctcaacaaagttgctgtttccttctaaggcaacttctgagctgagtcatagtgccaaatctgatgccggtttagtgggtggtggtgaagatggtgacactgatgatgatggtgatgatgatgatgacagagatagtgatggcttatccattcataagtgtatgtcatgctcatcctatagagaatcacaggaaaaggtaatgaatgattcagacacccacgaaaacagtcttatggatcagaataatccaatctcatactcactatctgagaattctgaagaagataatagagtcacaagtgtatcctcagacagtcaaactggtatggacagaagtcctggtaaatcaccatcagcaaatgggctatcccaaaagcacaatgatggaaaagaggaaaatgacattcagactggtagtgctctgcttcctctcagccctgaatctaaagcatgggcagttctgacaagtgatgaagaaagtggatcagggcaaggtacctcagatagccttaatgagaatgagacttccacagatttcagttttgcagacactaatgaaaaagatgctgatgggatcctggcagcaggtgactcagaaataactcctggattcccacagtccccaacatcatctgttactagcgagaactcagaagtgttccacgtttcagaggcagaggccagtaatagtagccatgagtctcgtattggtctagctgaggggttggaatccgagaagaaggcagttataccccttgtgatcgtgtcagccctgacttttatctgtctagtggttcttgtgggtattctcatctactggaggaaatgcttccagactgcacacttttacttagaggacagtacatcccctagagttatatccacacctccaacacctatctttccaatttcagatgatgtcggagcaattccaataaagcactttccaaagcatgttgcagatttacatgcaagtagtgggtttactgaagaatttgagacactgaaagagttttaccaggaagtgcagagctgtactgttgacttaggtattacagcagacagctccaaccacccagacaacaagcacaagaatcgatacataaatatcgttgcctatgatcatagcagggttaagctagcacagcttgctgaaaaggatggcaaactgactgattatatcaatgccaattatgttgatggctacaacagaccaaaagcttatattgctgcccaaggcccactgaaatccacagctgaagatttctggagaatgatatgggaacataatgtggaagttattgtcatgataacaaacctcgtggagaaaggaaggagaaaatgtgatcagtactggcctgccgatgggagtgaggagtacgggaactttctggtcactcagaagagtgtgcaagtgcttgcctattatactgtgaggaattttactctaagaaacacaaaaataaaaaagggctcccagaaaggaagacccagtggacgtgtggtcacacagtatcactacacgcagtggcctgacatgggagtaccagagtactccctgccagtgctgacctttgtgagaaaggcagcctatgccaagcgccatgcagtggggcctgttgtcgtccactgcagtgctggagttggaagaacaggcacatatattgtgctagacagtatgttgcagcagattcaacacgaaggaactgtcaacatatttggcttcttaaaacacatccgttcacaaagaaattatttggtacaaactgaggagcaatatgtcttcattcatgatacactggttgaggccatacttagtaaagaaactgaggtgctggacagtcatattcatgcctatgttaatgcactcctcattcctggaccagcaggcaaaacaaagctagagaaacaattccagctcctgagccagtcaaatatacagcagagtgactattctgcagccctaaagcaatgcaacagggaaaagaatcgaacttcttctatcatccctgtggaaagatcaagggttggcatttcatccctgagtggagaaggcacagactacatcaatgcctcctatatcatgggctattaccagagcaatgaattcatcattacccagcaccctctccttcataccatcaaggatttctggaggatgatatgggaccataatgcccaactggtggttatgattcctgatggccaaaacatggcagaagatgaatttgtttactggccaaataaagatgagcctataaattgtgagagctttaaggtcactcttatggctgaagaacacaaatgtctatctaatgaggaaaaacttataattcaggactttatcttagaagctacacaggatgattatgtacttgaagtgaggcactttcagtgtcctaaatggccaaatccagatagccccattagtaaaacttttgaacttataagtgttataaaagaagaagctgccaatagggatgggcctatgattgttcatgatgagcatggaggagtgacggcaggaactttctgtgctctgacaacccttatgcaccaactagaaaaagaaaattccgtggatgtttaccaggtagccaagatgatcaatctgatgaggccaggagtctttgctgacattgagcagtatcagtttctctacaaagtgatcctcagccttgtgagcacaaggcaggaagagaatccatccacctctctggacagtaatggtgcagcattgcctgatggaaatatagctgagagcttagagtctttagtttaacacagaaaggggtggggggactcacatctgagcattgttttcctcttcctaaaattaggcaggaaaatcagtctagttctgttatctgttgatttcccatcacctgacagtaactttcatgacataggattctgccgccaaatttatatcattaacaatgtgtgcctttttgcaagacttgtaatttacttattatgtttgaactaaaatgattgaattttacagtatttctaagaatggaattgtggtatttttttctgtattgattttaacagaaaatttcaatttatagaggttaggaattccaaactacagaaaatgtttgtttttagtgtcaaatttttagctgtatttgtagcaattatcaggtttgctagaaatataacttttaatacagtagcctgtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattgttcaaatggtttttatccaaggaattgcaaaaataaatataaatattgccattaaaaaaaaaaaaaaaaaaaaaaaaaaaaaaaaaaa

SEQ ID NO: 3 - protein, 168 aa, Homo sapiens
MQAQQYQQQRRKFAAAFLAFIFILAAVDTAEAGKKEKPEKKVKKSDCGEWQWSVCVPTSGDCGLGTREGTRTGAECKQTMKTQRCKIPCNWKKQFGAECKYQFQAWGECDLNTALKTRTGSLKRALHNAECQKTVTISKPCGKLTKPKPQAESKKKKKEGKKQEKMLD

SEQ ID NO: 4 - DNA, 1482 bp, Homo sapiens
attctccatttcccttccgttccctccctgtcagggcgtaattgagtcaaaggcaggatcaggttccccgccttccagtccaaaaatcccgccaagagagccccagagcagaggaaaatccaaagtggagagaggggaagaaagagaccagtgagtcatccgtccagaaggcggggagagcagcagcggcccaagcaggagctgcagcgagccgggtacctggactcagcggtagcaacctcgccccttgcaacaaaggcagactgagcgccagagaggacgtttccaactcaaaaatgcaggctcaacagtaccagcagcagcgtcgaaaatttgcagctgccttcttggcattcattttcatactggcagctgtggatactgctgaagcagggaagaaagagaaaccagaaaaaaaagtgaagaagtctgactgtggagaatggcagtggagtgtgtgtgtgcccaccagtggagactgtgggctgggcacacgggagggcactcggactggagctgagtgcaagcaaaccatgaagacccagagatgtaagatcccctgcaactggaagaagcaatttggcgcggagtgcaaataccagttccaggcctggggagaatgtgacctgaacacagccctgaagaccagaactggaagtctgaagcgagccctgcacaatgccgaatgccagaagactgtcaccatctccaagccctgtggcaaactgaccaagcccaaacctcaagcagaatctaagaagaagaaaaaggaaggcaagaaacaggagaagatgctggattaaaagatgtcacctgtggaacataaaaaggacatcagcaaacaggatcagttaactattgcatttatatgtaccgtaggctttgtattcaaaaattatctatagctaagtacacaataagcaaaaacaaaaagaaaagaaaatttttgtagtagcgttttttaaatgtatactatagtaccagtaggggcttataataaaggactgtaatcttatttaggaagttgacttatagtacatgataaatgatagacaattgaggtaagttttttgaaattatgtgacattttacattaaattttttttacattttttgggcagcaatttaaatgttatgactatgtaaactacttctcttgttaggtaatttttttcacctagatttttttcccaattgagaaaaatatatactaaacaaaatagcaataaaacataatcactctatttgaagaaaatatcttgttttctgccaatagattttttaaaatgtagtcagcaaaatgggggtggggaagcagagcatgtcctagttcaatgttgactttttttttttttaaagaaaagcattaagacataaaattctttcactttggcagaagcatttgttttcttgatgaaattatttttccatctgaggaaaaaaatactaggaaaataaatcaaggtgatgctgaaaaaaaaa

SEQ ID NO: 5 - DNA, 57 bp, Artificial sequence
  synthetic sequence
ccggccagacaacaagcacaagaatctcgagattcttgtgcttgttgtctggttttt

SEQ ID NO: 6 - DNA, 57 bp, Artificial sequence
  synthetic sequence
ccggaggcaagaaacaggagaagatctcgagatcttctcctgtttcttgcctttttt